(19) United States
(12) Patent Application Publication
(10) Pub. No.: US 2024/0000075 A1
(43) Pub. Date: Jan. 4, 2024

(54) FUNGICIDAL ARYL AMIDINES

(71) Applicant: CORTEVA AGRISCIENCE LLC, INDIANAPOLIS, IN (US)

(72) Inventors: CRUZ AVILA-ADAME, CARMEL, IN (US); VASUDEV R. BHONDE, CARMEL, IN (US); SUSANA LOPEZ, CARMEL, IN (US); BRIAN LOY, INDIANAPOLIS, IN (US); STACY T. MEYER, ZIONSVILLE, IN (US); ALEX NOLAN, INDIANAPOLIS, IN (US); ADRIAN TLAHUEXT-ACA, INDIANAPOLIS, IN (US)

(73) Assignee: CORTEVA AGRISCIENCE LLC, INDIANAPOLIS, IN (US)

(21) Appl. No.: 18/253,808

(22) PCT Filed: Nov. 22, 2021
(86) PCT No.: PCT/US2021/060244

§ 371 Date: May 22, 2023

Related U.S. Application Data
(60) Provisional application No. 63/117,145, filed on Nov. 23, 2020.

(51) Int. Cl. A01N 37/52; A01P 3/00; C07C 257/12; C07D 295/12; C07C 335/30; C07D 295/145; C07D 211/98; C07C 257/14; A01N 43/36; A01N 43/40; A01N 43/84; A01N 47/42

(52) U.S. Cl. CPC A01N 37/52; A01P 3/00; C07C 257/12; C07D 295/12; C07C 335/30; C07D 295/145; C07D 211/98; C07C 257/14; A01N 43/36; A01N 43/40; A01N 43/84; A01N 47/42; C07C 2601/14; C07C 2601/08; C07C 2601/04; C07C 2602/08; C07C 2601/02

(57) ABSTRACT

This disclosure relates to aryl amidines of Formula I and their use as fungicides.

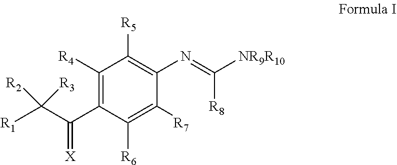

### CROSS REFERENCE TO RELATED APPLICATIONS

This application claims the benefit of U.S. Provisional Patent Application Ser. No. 63/117,145 filed Nov. 23, 2020, which is expressly incorporated by reference herein.

### BACKGROUND & SUMMARY

Fungicides are compounds, of natural or synthetic origin, which act to protect and/or cure plants against damage caused by agriculturally relevant fungi. Generally, no single fungicide is useful in all situations. Consequently, research is ongoing to produce fungicides that may have better performance, are easier to use, and cost less.

The present disclosure relates to aryl amidines and their use as fungicides. The compounds of the present disclosure may offer protection against ascomycetes, basidiomycetes, deuteromycetes and oomycetes.

One embodiment of the present disclosure may include compounds of Formula I.

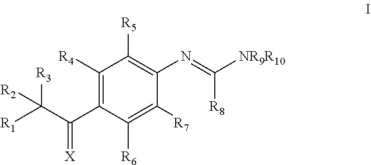

wherein
R₁ is selected from the group consisting of C₂-C₈ alkyl, C₁-C₈ haloalkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, C₃-C₈ heterocycloalkyl, C₃-C₈ substituted heterocycloalkyl, C₅-C₇ heteroaryl, C₅-C₇ substituted heteroaryl, aryl, substituted aryl;
each R₂ and R₃ independently is selected from the group consisting of hydrogen, C₁-C₈ alkyl, C₁-C₈ haloalkyl, C₂-C₈ alkenyl, C₂-C₈ substituted alkenyl, C₂-C₈ alkynyl, C₂-C₈ substituted alkynyl;
or R₁ and R₂ may be covalently bonded together to form a C₄-C₈ cycloalkyl group, C₃-C₈ substituted cycloalkyl group, C₃-C₈ heterocycloalkyl, or C₃-C₈ substituted heterocycloalkyl group;
each R₄, R₅, and R₆ independently is selected from the group consisting of hydrogen, halogen, cyano, nitro, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, and C₁-C₈ substituted alkoxy;
R₇ is H;
R₈ is selected from the group consisting of hydrogen, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, C₁-C₈ substituted alkoxy, and thiol;
or R₈ and R₉ may be covalently bonded together to form a C₃-C₈ heterocycloalkyl or C₃-C₈ substituted heterocycloalkyl group;
each R₉ and R₁₀ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, aryl, substituted aryl, C₁-C₈ alkylaryl, and substituted C₁-C₈ alkylaryl;
or R₉ and R₁₀ may be covalently bonded together to form a C₃-C₈ heterocycloalkyl or C₃-C₈ substituted heterocycloalkyl group;
X is O;
wherein any and all heterocyclic rings may contain up to three heteroatoms selected from the group consisting of O, N, and S;
or a tautomer or salt thereof.

Another embodiment of the present disclosure may include a fungicidal composition for the control or prevention of fungal attack comprising the compounds described above and a phytologically acceptable carrier material.

Yet another embodiment of the present disclosure may include a method for the control or prevention of fungal attack on a plant, the method including the steps of applying a fungicidally effective amount of one or more of the compounds described above to at least one of the fungus, a seed, the plant, and an area adjacent to the plant.

It will be understood by those skilled in the art that the following terms may include generic “R”-groups within their definitions, e.g., “the term alkoxy refers to an —OR substituent”. It is also understood that within the definitions for the following terms, these “R” groups are included for illustration purposes and should not be construed as limiting or being limited by substitutions about Formula I.

The term “alkyl” refers to a branched, unbranched, or saturated acyclic substituent consisting of carbon and hydrogen atoms including, but not limited to, methyl, ethyl, propyl, butyl, isopropyl, isobutyl, tertiary butyl, pentyl, hexyl, and the like.

The term “alkenyl” refers to an acyclic, unsaturated (at least one carbon-carbon double bond), branched or unbranched, substituent consisting of carbon and hydrogen, including, but not limited to, ethenyl, propenyl, butenyl, isopropenyl, isobutenyl, and the like.

The term “alkynyl” refers to an acyclic, unsaturated (at least one carbon-carbon triple bond), branched or unbranched, substituent consisting of carbon and hydrogen, for example, ethynyl, propargyl, butynyl, and pentynyl.

The term “cycloalkenyl” refers to a monocyclic or polycyclic, unsaturated (at least one carbon-carbon double bond) substituent consisting of carbon and hydrogen, for example, cyclobutenyl, cyclopentenyl, cyclohexenyl, norbornenyl, bicyclo[2.2.2]octenyl, tetrahydronaphthyl, hexahydronaphthyl, and octahydronaphthyl.

The term “cycloalkyl” refers to a monocyclic or polycyclic, saturated substituent consisting of carbon and hydrogen, for example, cyclopropyl, cyclobutyl, cyclopentyl, norbornyl, bicyclo[2.2.2]octyl, and decahydronaphthyl.

The terms “aryl” and “Ar” refer to any aromatic ring, mono- or bi-cyclic, containing 0 heteroatoms, for example phenyl and naphthyl.

The terms “heteroaryl” refers to any aromatic ring, mono- or bi-cyclic, containing 1 or more heteroatoms, for example pyridinyl, pyrimidinyl, furanyl, and thiophenyl.

The term “heterocycloalkyl” refers to any saturated, non-aromatic, mono- or bi-cyclic ring, containing carbon and hydrogen atoms and one or more heteroatoms.

The terms “alkylaryl”, “alkylheteroaryl”, “alkylcycloalkyl”, and “alkylheterocycloalkyl” refer to an alkyl group as defined herein substituted with an aryl group, a heteroaryl group, a cycloalkyl group, or a heterocycloalkyl group, respectively, as defined herein.

The term “alkoxy” refers to an —OR substituent.

The term “cyano” refers to a —C≡N substituent.

The term “amino” refers to an —N(R)₂ substituent.

The term “halogen” or “halo” refers to one or more halogen atoms, defined as F, Cl, Br, and I.

The term “nitro” refers to a —NO₂ substituent.

The term “thiol” refers to a —SH substituent.

The term “haloalkyl” means an alkyl further consisting of, from one to the maximum possible number of, identical or different, halogens, for example, fluoromethyl, trifluoromethyl, 2,2-difluoropropyl, chloromethyl, trichloromethyl, and 1,1,2,2-tetrafluoroethyl.

The terms “ambient temperature” and “room temperature” refer to temperatures ranging from about 20° C. to about 24° C.

Throughout the disclosure, reference to the compounds of Formula I is read as also including all stereoisomers, for example diastereomers, enantiomers, and mixtures thereof. In another embodiment, Formula I is read as also including salts or hydrates thereof. Exemplary salts include, but are not limited to: hydrochloride, hydrobromide, hydroiodide, trifluoroacetate, and trifluoromethane sulfonate.

It is also understood by those skilled in the art that additional substitution is allowable, unless otherwise noted, as long as the rules of chemical bonding and strain energy are satisfied and the product still exhibits fungicidal activity.

Another embodiment of the present disclosure is a use of a compound of Formula I, for protection of a plant against attack by a phytopathogenic organism or the treatment of a plant infested by a phytopathogenic organism, comprising the application of a compound of Formula I, or a composition comprising the compound to soil, a plant, a part of a plant, foliage, and/or roots.

Additionally, another embodiment of the present disclosure is a composition useful for protecting a plant against attack by a phytopathogenic organism and/or treatment of a plant infested by a phytopathogenic organism comprising a compound of Formula I and a phytologically acceptable carrier material.

### DETAILED DESCRIPTION

The compounds of the present disclosure may be applied by any of a variety of known techniques, either as the compounds or as formulations comprising the compounds. For example, the compounds may be applied to the roots or foliage of plants for the control of various fungi, without damaging the commercial value of the plants. The materials may be applied in the form of any of the generally used formulation types, for example, as solutions, dusts, wettable powders, flowable concentrates, or emulsifiable concentrates.

Preferably, the compounds of the present disclosure are applied in the form of a formulation, comprising one or more of the compounds of Formula I with a phytologically acceptable carrier. Concentrated formulations may be dispersed in water, or other liquids, for application, or formulations may be dust-like or granular, which may then be applied without further treatment. The formulations can be prepared according to procedures that are conventional in the agricultural chemical art.

The present disclosure contemplates all vehicles by which one or more of the compounds may be formulated for delivery and used as a fungicide. Typically, formulations are applied as aqueous suspensions or emulsions. Such suspensions or emulsions may be produced from water-soluble, water-suspendible, or emulsifiable formulations which are solids, usually known as wettable powders; or liquids, usually known as emulsifiable concentrates, aqueous suspensions, or suspension concentrates. As will be readily appreciated, any material to which these compounds may be added may be used, provided it yields the desired utility without significant interference with the activity of these compounds as antifungal agents.

Wettable powders, which may be compacted to form water-dispersible granules, comprise an intimate mixture of one or more of the compounds of Formula I, an inert carrier and surfactants. The concentration of the compound in the wettable powder may be from about 10 percent to about 90 percent by weight based on the total weight of the wettable powder, more preferably about 25 weight percent to about 75 weight percent. In the preparation of wettable powder formulations, the compounds may be compounded with any finely divided solid, such as prophyllite, talc, chalk, gypsum, Fuller's earth, bentonite, attapulgite, starch, casein, gluten, montmorillonite clays, diatomaceous earths, purified silicates or the like. In such operations, the finely divided carrier and surfactants are typically blended with the compound(s) and milled.

Emulsifiable concentrates of the compounds of Formula I may comprise a convenient concentration, such as from about 1 weight percent to about 50 weight percent of the compound, in a suitable liquid, based on the total weight of the concentrate. The compounds may be dissolved in an inert carrier, which is either a water-miscible solvent or a mixture of water-immiscible organic solvents, and emulsifiers. The concentrates may be diluted with water and oil to form spray mixtures in the form of oil-in-water emulsions. Useful organic solvents include aromatics, especially the high-boiling naphthalenic and olefinic portions of petroleum such as heavy aromatic naphtha. Other organic solvents may also be used, for example, terpenic solvents, including rosin derivatives, aliphatic ketones, such as cyclohexanone, and complex alcohols, such as 2-ethoxyethanol.

Emulsifiers which may be advantageously employed herein may be readily determined by those skilled in the art and include various nonionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters solubilized with the polyol or polyoxyalkylene. Cationic emulsifiers include quaternary ammonium compounds and fatty amine salts. Anionic emulsifiers include the oil-soluble salts (e.g., calcium) of alkylaryl sulfonic acids, oil-soluble salts or sulfated polyglycol ethers and appropriate salts of phosphated-polyglycol ether.

Representative organic liquids which may be employed in preparing the emulsifiable concentrates of the compounds of the present disclosure are the aromatic liquids such as xylene, propyl benzene fractions; or mixed naphthalene fractions, mineral oils, substituted aromatic organic liquids such as dioctyl phthalate; kerosene; dialkyl amides of various fatty acids, particularly the dimethyl amides of fatty glycols and glycol derivatives such as the n-butyl ether, ethyl ether or methyl ether of diethylene glycol, the methyl ether of triethylene glycol, petroleum fractions or hydrocarbons such as mineral oil, aromatic solvents, paraffinic oils, and the like; vegetable oils such as soybean oil, rapeseed oil, olive oil, castor oil, sunflower seed oil, coconut oil, corn oil, cottonseed oil, linseed oil, palm oil, peanut oil, safflower oil, sesame oil, tung oil and the like; esters of the above vegetable oils; and the like. Mixtures of two or more organic liquids may also be employed in the preparation of the emulsifiable concentrate. Organic liquids include xylene, and propyl benzene fractions, with xylene being most preferred in some cases. Surface-active dispersing agents are typically employed in liquid formulations and in an amount of from 0.1 to 20 percent by weight based on the combined weight of the dispersing agent with one or more of the compounds. The formulations can also contain other compatible additives, for example, plant growth regulators and other biologically active compounds used in agriculture.

Aqueous suspensions comprise suspensions of one or more water-insoluble compounds of Formula I, dispersed in an aqueous vehicle at a concentration in the range from about 1 to about 50 weight percent, based on the total weight of the aqueous suspension. Suspensions are prepared by finely grinding one or more of the compounds, and vigorously mixing the ground material into a vehicle comprised of water and surfactants chosen from the same types discussed above. Other components, such as inorganic salts and synthetic or natural gums, may also be added to increase the density and viscosity of the aqueous vehicle.

The compounds of Formula I can also be applied as granular formulations, which are particularly useful for applications to the soil. Granular formulations generally contain from about 0.5 to about 10 weight percent, based on the total weight of the granular formulation of the compound(s), dispersed in an inert carrier which consists entirely or in large part of coarsely divided inert material such as attapulgite, bentonite, diatomite, clay or a similar inexpensive substance. Such formulations are usually prepared by dissolving the compounds in a suitable solvent and applying it to a granular carrier which has been preformed to the appropriate particle size, in the range of from about 0.5 to about 3 millimeters (mm). A suitable solvent is a solvent in which the compound is substantially or completely soluble. Such formulations may also be prepared by making a dough or paste of the carrier and the compound and solvent, and crushing and drying to obtain the desired granular particle.

Dusts containing the compounds of Formula I may be prepared by intimately mixing one or more of the compounds in powdered form with a suitable dusty agricultural carrier, such as, for example, kaolin clay, ground volcanic rock, and the like. Dusts can suitably contain from about 1 to about 10 weight percent of the compounds, based on the total weight of the dust.

The formulations may additionally contain adjuvant surfactants to enhance deposition, wetting, and penetration of the compounds onto the target crop and organism. These adjuvant surfactants may optionally be employed as a component of the formulation or as a tank mix. The amount of adjuvant surfactant will typically vary from 0.01 to 1.0 percent by volume, based on a spray-volume of water, preferably 0.05 to 0.5 volume percent. Suitable adjuvant surfactants include, but are not limited to ethoxylated nonyl phenols, ethoxylated synthetic or natural alcohols, salts of the esters or sulfosuccinic acids, ethoxylated organosilicones, ethoxylated fatty amines, blends of surfactants with mineral or vegetable oils, crop oil concentrate (mineral oil (85%)+emulsifiers (15%)); nonylphenol ethoxylate; benzylcocoalkyldimethyl quaternary ammonium salt; blend of petroleum hydrocarbon, alkyl esters, organic acid, and anionic surfactant; C₉-C₁₁ alkylpolyglycoside; phosphated alcohol ethoxylate; natural primary alcohol (C₁₂-C₁₆) ethoxylate; di-sec-butylphenol EO-PO block copolymer; polysiloxane-methyl cap; nonylphenol ethoxylate+urea ammonium nitrate; emulsified methylated seed oil; tridecyl alcohol (synthetic) ethoxylate (8EO); tallow amine ethoxylate (15 EO); PEG(400) dioleate-99. The formulations may also include oil-in-water emulsions such as those disclosed in U.S. patent application Ser. No. 11/495,228, the disclosure of which is expressly incorporated by reference herein.

Another embodiment of the present disclosure is a method for the control or prevention of fungal attack. This method comprises applying to the soil, plant, roots, foliage, or locus of the fungus, or to a locus in which the infestation is to be prevented (for example applying to cereal or grape plants), a fungicidally effective amount of one or more of the compounds of Formula I. The compounds are suitable for treatment of various plants at fungicidal levels, while exhibiting low phytotoxicity. The compounds may be useful both in a protectant and/or an eradicant fashion.

The compounds have been found to have significant fungicidal effect particularly for agricultural use. Many of the compounds are particularly effective for use with agricultural crops and horticultural plants.

It will be understood by those skilled in the art that the efficacy of the compound for the foregoing fungi establishes the general utility of the compounds as fungicides.

The compounds have broad ranges of activity against fungal pathogens. Exemplary pathogens may include, but are not limited to, the causative agent of Septoria leaf blotch of wheat (Zymoseptoria tritici), spot blotch of barley (Cochliobolus sativus), wheat brown rust (Puccinia triticina), wheat stripe rust (Puccinia striiformis), scab of apple (Venturia inaequalis), blister smut of maize (Ustilago maydis), powdery mildew of grapevine (Uncinula necator), leaf blotch of barley (Rhynchosporium commune), blast of rice (Magnaporthe grisea), Asian soybean rust (Phakopsora pachyrhizi), glume blotch of wheat (Parastagonospora nodorum), Anthracnose of cucurbits (Glomerella lagenarium), leaf spot of beet (Cercospora beticola), early blight of tomato (Alternaria solani), net blotch of barley (Pyrenophora teres), powdery mildew of wheat (Blumeria graminis f sp. tritici), powdery mildew of barley (Blumeria graminis f sp. hordei), powdery mildew of cucurbits (Erysiphe cichoracearum), sudden death syndrome of soybean (Fusarium virguliforme), collar rot or damping-off of seedlings (Rhizoctonia solani), root rot (Pythium ultimum), grey mold (Botrytis cinerea), Ramularia leaf spot (Ramularia collo-cygni), tan spot of wheat (Pyrenophora tritici-repentis), Northern leaf blight of maize (Exserohilum turcicum), Southern rust of maize (Puccinia polysora), white mold (Sclerotinia sclerotiorum), powdery mildew of soybean (Erysiphe diffusa), head blight of cereals (Fusarium graminearum), powdery mildew of apple (Podosphaera leucotricha), Anthracnose of soybean (Colletotrichum truncatum), Cercospora leaf blight (Cercospora kikuchii), frogeye leaf spot (Cerospora sojina), target spot of soybean (Corynespora cassiicola), and leaf spot of soybean (Septoria glycines). The exact amount of the active material to be applied is dependent not only on the specific active material being applied, but also on the particular action desired, the fungal species to be controlled, and the stage of growth thereof, as well as the part of the plant or other product to be contacted with the compound. Thus, all the compounds, and formulations containing the same, may not be equally effective at similar concentrations or against the same fungal species.

The compounds are effective in use with plants in a disease-inhibiting and phytologically acceptable amount. The term “disease-inhibiting and phytologically acceptable amount” refers to an amount of a compound that kills or inhibits the plant disease for which control is desired, but is not significantly toxic to the plant. This amount will generally be from about 0.1 to about 1000 ppm (parts per million), with 1 to 500 ppm being preferred. The exact concentration of compound required varies with the fungal disease to be controlled, the type of formulation employed, the method of application, the particular plant species, climate conditions, and the like. A suitable application rate is typically in the range from about 0.10 to about 4 pounds per acre (about 0.01 to 0.45 grams per square meter, g/m²).

Any range or desired value given herein may be extended or altered without losing the effects sought, as is apparent to the skilled person for an understanding of the teachings herein.

The compounds of Formula I may be made using well-known chemical procedures. Intermediates not specifically mentioned in this disclosure are either commercially available, may be made by routes disclosed in the chemical literature, or may be readily synthesized from commercial starting materials utilizing standard procedures.

### General Schemes

The following schemes illustrate approaches to generating aryl amidine compounds of Formula I. The following descriptions and examples are provided for illustrative purposes and should not be construed as limiting in terms of substituents or substitution patterns.

Compounds of Formula 1.3, wherein R₄, R₅, R₆ and R₇ are as originally defined, and Y is an alkyl group, can be prepared by the method shown in Scheme 1, step a. The compound of Formula 1.1, wherein R₄, R₅, R₆ and R₇ are as originally defined, can be treated with a protecting group, such as a chloroformate compound of Formula 1.2, wherein Y is an alkyl group, such as methyl or ethyl, in the presence of a base, such as potassium carbonate (K₂CO₃), in a solvent, such as acetone, at a temperature from about ambient temperature to about reflux (˜56° C.), to afford compounds of Formula 1.3, wherein R₄, R₅, R₆ and R₇ are as originally defined, and Y is an alkyl group, as shown in step a.

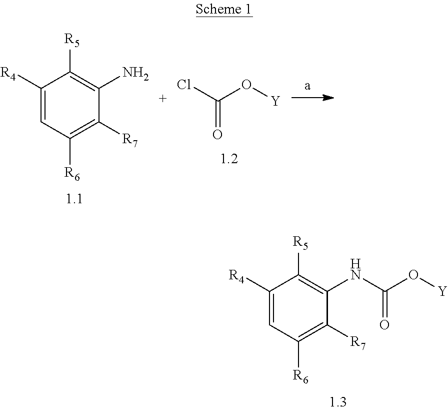

Compounds of Formula 2.2, wherein R₁, R₂, R₃, R₄, R₅, R₆ and R₇ are as originally defined, and Y is an alkyl group, can be prepared by the method shown in Scheme 2, step b. The compound of Formula 1.3, wherein R₄, R₅, R₆ and R₇ are as originally defined, and Y is an alkyl group, can be treated with an acid chloride, such as a compound of Formula 2.1, wherein R₁, R₂ and R₃ are as originally defined, in the presence of a metal reagent, such as zinc(II) chloride (ZnCl₂) or aluminum(III) chloride (AlC₃), in a solvent, such as dichloromethane (DCM) or 1,2-dichloroethane (DCE), at a temperature from about ambient temperature to about reflux (˜40° C. or ˜83° C., respectively), to afford compounds of Formula 2.2, wherein R₁, R₂, R₃, R₄, R₅, R₆ and R₇ are as originally defined, and Y is an alkyl group, as shown in step b.

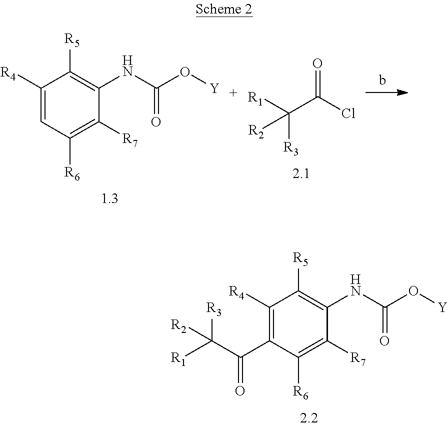

Alternatively, compounds of Formula 2.2, wherein R₁, R₂, R₃, R₄, R₅, R₆ and R₇ are as originally defined, and Y is an alkyl group, can be prepared by the method shown in Scheme 3, step c. The compound of Formula 1.3, wherein R₄, R₅, R₆ and R₇ are as originally defined, and Y is an alkyl group, can be treated with a carboxylic acid, such as a compound of Formula 3.1, wherein R₁, R₂ and R₃ are as originally defined, in the presence of a chlorinating agent, such as oxalyl chloride ((COCl)₂) or thionyl chloride (SOCl₂), and an activator, such as N,N-dimethylformamide (DMF), in the presence of a metal reagent, such as zinc(II) chloride (ZnCl₂) or aluminum(III) chloride (AlCl₃), in a solvent, such as dichloromethane (DCM) or 1,2-dichloroethane (DCE), at a temperature from about ambient temperature to about reflux (˜40° C. or ˜83° C., respectively), to afford compounds of Formula 2.2, wherein R₁, R₂, R₃, R₄, R₅, R₆ and R₇ are as originally defined, and Y is an alkyl group, as shown in step c.

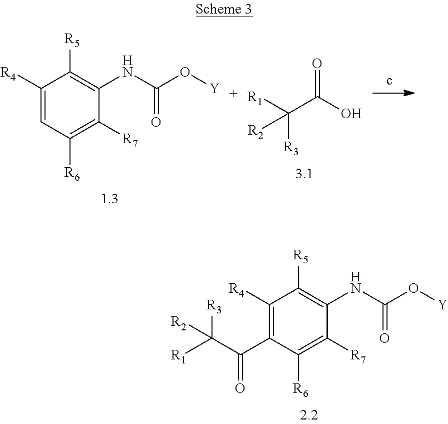

Compounds of Formula 4.2, wherein R₂, R₃, R₄, R₅, R₆ and R₇ are as originally defined, and Y is an alkyl group, can be prepared by the method shown in Scheme 4, step d. The compound of Formula 1.3, wherein R₄, R₅, R₆ and R₇ are as originally defined, and Y is an alkyl group, can be treated with an acid chloride, such as a compound of Formula 4.1, wherein R₂ and R₃ are as originally defined, in the presence of a metal reagent, such as aluminum(III) chloride (AlCl₃), in a solvent, such as dichloromethane (DCM) or 1,2-dichloroethane (DCE), at a temperature from about ambient temperature to about reflux (˜40° C. or ˜83° C., respectively), to afford compounds of Formula 4.2, wherein R₂, R₃, R₄, R₅, R₆ and R₇ are as originally defined, and Y is an alkyl group, as shown in step d. Alternatively, compounds of Formula 4.2, wherein R₂, R₃, R₄, R₅, R₆ and R₇ are as originally defined, and Y is an alkyl group, can be prepared by the method shown in Scheme 4, step e. The compound of Formula 1.3, wherein R₄, R₅, R₆ and R₇ are as originally defined, and Y is an alkyl group, can be treated with a carboxylic acid, such as a compound of Formula 4.3, wherein R₂ and R₃ are as originally defined, in the presence of a chlorinating agent, such as oxalyl chloride ((COCl)₂) or thionyl chloride (SOCl₂), and an activator, such as N,N-dimethylformamide (DMF), in the presence of a metal reagent, such as aluminum(III) chloride (AlCl₃), in a solvent, such as dichloromethane (DCM) or 1,2-dichloroethane (DCE), at a temperature from about ambient temperature to about reflux (˜40° C. or ˜83° C., respectively), to afford compounds of Formula 4.2, wherein R₂, R₃, R₄, R₅, R₆ and R₇ are as originally defined, and Y is an alkyl group, as shown in step e.

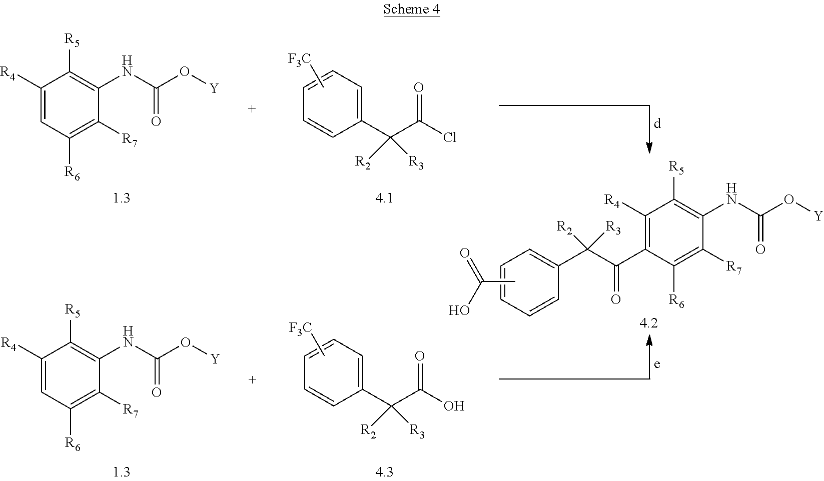

Compounds of Formula 5.1, wherein R₁, R₂, R₃, R₄, R₅, R₆ and R₇ are as originally defined, can be prepared by the method shown in Scheme 5, step f. The compound of Formula 2.2, wherein R₁, R₂, R₃, R₄, R₅, R₆ and R₇ are as originally defined, and Y is an alkyl group, can be treated with a base, such as sodium hydroxide (NaOH), in a solvent, such as ethanol (EtOH), at a temperature from about ambient temperature to about reflux (˜78° C.), to afford compounds of Formula 5.1, wherein R₁, R₂, R₃, R₄, R₅, R₆ and R₇ are as originally defined, as shown in step f.

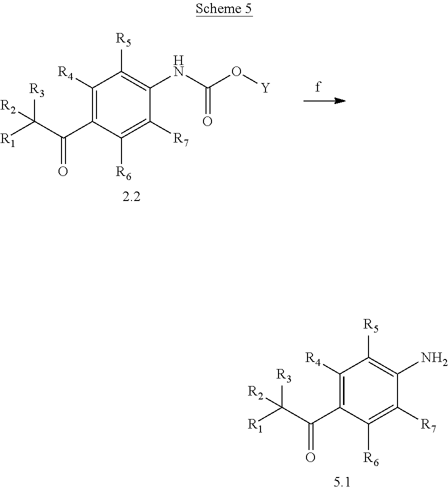

Compounds of Formula 6.2, wherein R₂, R₃, R₄, R₅, R₆ and R₇ are as originally defined, can be prepared by the method shown in Scheme 6, steps g-h. Compounds of Formula 6.1, wherein R₂, R₃, R₄, R₅, R₆ and R₇ are as originally defined, can be prepared by the method shown in Scheme 6, step g. The compound of Formula 4.2, wherein R₂, R₃, R₄, R₅, R₆ and R₇ are as originally defined, and Y is an alkyl group, can be treated with a base, such as sodium hydroxide (NaOH), in a solvent mixture, such as 1:1 tetrahydrofuran (THF):methanol (MeOH), at a temperature from about ambient temperature to about reflux (˜60° C.), to afford compounds of Formula 6.1, wherein R₂, R₃, R₄, R₅, R₆ and R₇ are as originally defined, as shown in step g. Compounds of Formula 6.2, wherein R₂, R₃, R₄, R₅, R₆ and R₇ are as originally defined, can be prepared by the method shown in Scheme 6, step h. The compound of Formula 6.1, wherein R₂, R₃, R₄, R₅, R₆ and R₇ are as originally defined, can be treated with a methylating agent, such as trimethylsilyldiazomethane, in a solvent such as methanol (MeOH), at a temperature of about ambient temperature, to afford compounds of Formula 6.2, wherein R₂, R₃, R₄, R₅, R₆ and R₇ are as originally defined, as shown in step h.

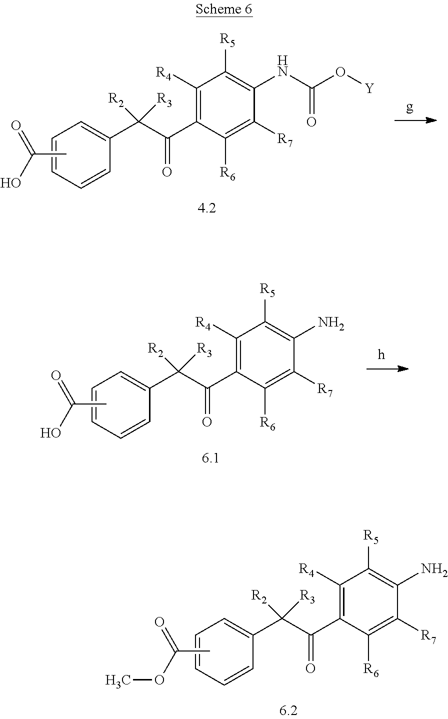

Compounds of Formula 7.3, wherein R₁, R₂, R₃, R₄, R₅, R₆, R₇, R₉ and R₁₀ are as originally defined, can be prepared by the method shown in Scheme 7, steps i-j. Compounds of Formula 7.1, wherein R₁, R₂, R₃, R₄, R₅, R₆ and R₇ are as originally defined, and Z is an alkyl group, can be prepared by the method shown in Scheme 7, step i. The compound of Formula 5.1, wherein R₁, R₂, R₃, R₄, R₅, R₆ and R₇ are as originally defined, can be treated with a trialkyl orthoformate (CH(OZ)₃), such as trimethyl orthoformate or triethyl orthoformate, in the presence of an acid catalyst, such asp-toluenesulfonic acid monohydrate (pTsOH·H₂O), at a temperature from about ambient temperature to about reflux (˜100° C. or ˜140° C., respectively), to afford compounds of Formula 7.1, wherein R₁, R₂, R₃, R₄, R₅, R₆ and R₇ are as originally defined, and Z is an alkyl group, as shown in step i. Compounds of Formula 7.3, wherein R₁, R₂, R₃, R₄, R₅, R₆, R₇, R₉ and R₁₀ are as originally defined, can be prepared by the method shown in Scheme 7, step j. The compound of Formula 7.1, wherein R₁, R₂, R₃, R₄, R₅, R₆ and R₇ are as originally defined, and Z is an alkyl group, can be treated with an amine, such as a compound of Formula 7.2, wherein R₉ and R₁₀ are as originally defined, in a solvent, such as DCM, at a temperature from about ambient temperature to about reflux (˜40° C.), to afford compounds of Formula 7.3, wherein R₁, R₂, R₃, R₄, R₅, R₆, R₇, R₉ and R₁₀ are as originally defined, as shown in step j.

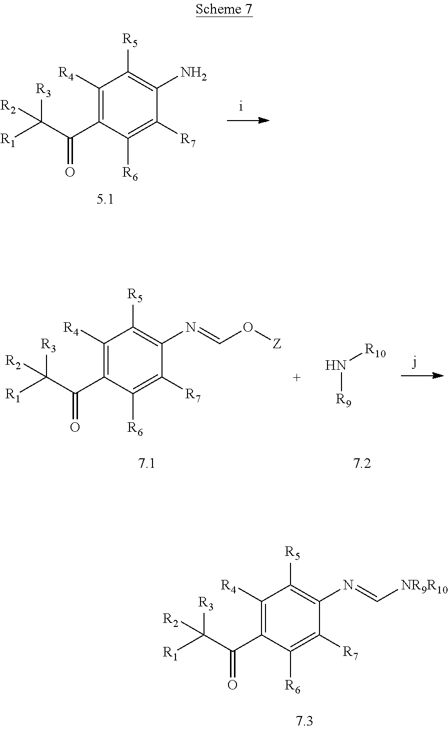

Alternatively, compounds of Formula 7.3, wherein R₁, R₂, R₃, R₄, R₅, R₆, R₇, R₉ and R₁₀ are as originally defined, can be prepared by the method shown in Scheme 8, step k. The compound of Formula 5.1, wherein R₁, R₂, R₃, R₄, R₅, R₆ and R₇ are as originally defined, can be treated with an amine, such as a compound of Formula 8.1, wherein R₉ and R₁₀ are as originally defined, in a solvent, such as toluene, at a temperature from about ambient temperature to about reflux (˜111° C.), to afford compounds of Formula 7.3, wherein R₁, R₂, R₃, R₄, R₅, R₆, R₇, R₉ and R₁₀ are as originally defined, as shown in step k.

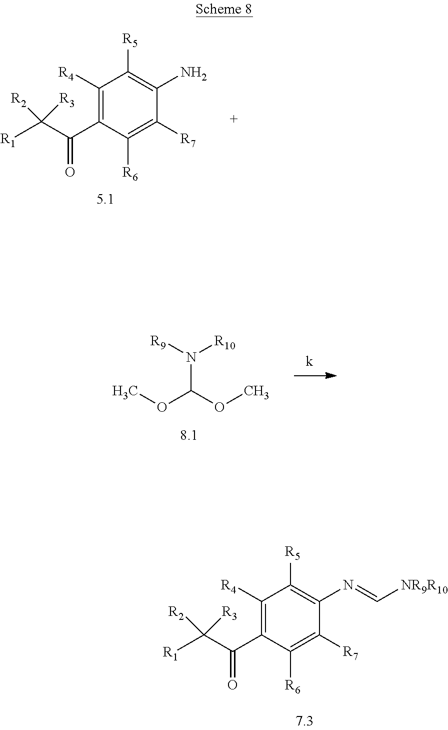

Compounds of Formula 9.2, wherein R₁, R₂, R₃, R₄, R₅, R₆, R₇, R₈, R₉ and R₁₀ are as originally defined, can be prepared by the method shown in Scheme 9, step 1. The compound of Formula 5.1, wherein R₁, R₂, R₃, R₄, R₅, R₆ and R₇ are as originally defined, can be treated with an amide, such as a compound of Formula 9.1, wherein R₈, R₉ and R₁₀ are as originally defined, in the presence of a dehydrating reagent, such as oxalyl chloride ((COCl)₂) or phosphoryl trichloride (POCl₃), in a solvent such as DCM or toluene, at a temperature from about ambient temperature to about reflux (˜40° C. or ˜111° C., respectively), to afford compounds of Formula 9.2, wherein R₁, R₂, R₃, R₄, R₅, R₆, R₇, R₈, R₉ and R₁₀ are as originally defined, as shown in step 1.

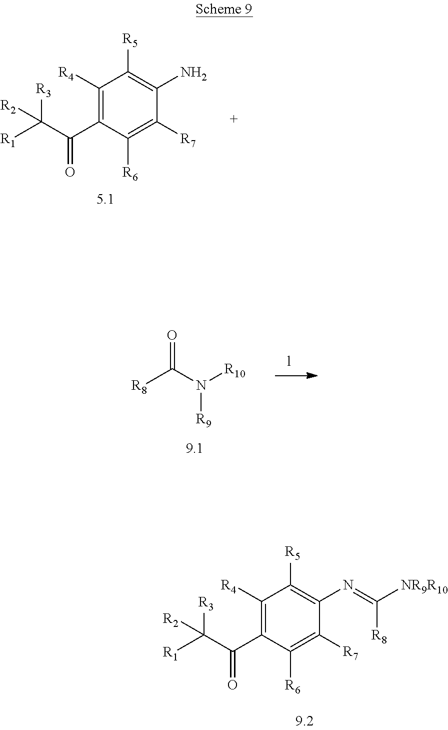

Compounds of Formula 10.2, wherein R₁, R₂, R₃, R₄, R₅, R₆, R₇, R₉ and R₁₀ are as originally defined, can be prepared by the method shown in Scheme 10, steps m-n. Compounds of Formula 10.1, wherein R₁, R₂, R₃, R₄, R₅, R₆ and R₇ are as previously defined, can be prepared by the method shown in Scheme 10, step m. The compound of Formula 5.1, wherein R₁, R₂, R₃, R₄, R₅, R₆ and R₇ are as previously defined, can be treated with thiophosgene, in the presence of a base such as sodium bicarbonate (NaHCO₃), in a solvent mixture such as 1:1 DCM:water (H₂O), at a temperature of about ambient temperature to afford compounds of Formula 10.1, wherein R₁, R₂, R₃, R₄, R₅, R₆ and R₇ are as previously defined, as shown in step m. Compounds of Formula 10.2, wherein R₁, R₂, R₃, R₄, R₅, R₆, R₇, R₉ and R₁₀ are as originally defined, can be prepared by the method shown in Scheme 10, step n. The compound of Formula 10.1, wherein R₁, R₂, R₃, R₄, R₅, R₆ and R₇ are as previously defined, can be treated with an amine, such as a compound of Formula 7.2, wherein R₉ and R₁₀ are as originally defined, in a solvent such as DCM, at a temperature of about ambient temperature to afford compounds of Formula 10.2, wherein R₁, R₂, R₃, R₄, R₅, R₆, R₇, R₉ and R₁₀ are as originally defined, as shown in step n.

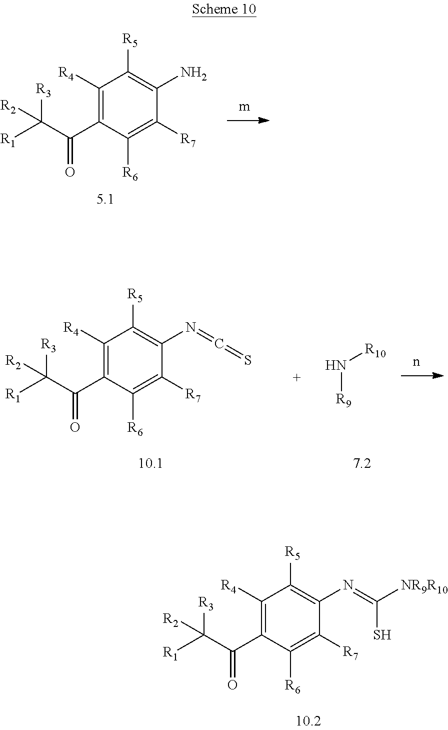

Compounds of Formula 9.2, wherein R₁, R₂, R₃, R₄, R₅, R₆, R₇, R₈, R₉ and R₁₀ are as originally defined, can also be prepared by the method shown in Scheme 11, steps o-r. Compounds of Formula 11.2, wherein R₄, R₅, R₆ and R₇ are as previously defined, can be prepared by the method shown in Scheme 11, step o. The compound of Formula 11.1, wherein R₄, R₅, R₆ and R₇ are as previously defined, can be treated with a halogenating reagent, such as N-bromosuccinimide (NBS), in a solvent such as N,N-dimethylformamide (DMF) or acetonitrile, at a temperature from about 0° C. to about ambient temperature, to afford compounds of Formula 11.2, wherein R₄, R₅, R₆ and R₇ are as previously defined, as shown in step o. Compounds of Formula 11.3, wherein R₄, R₅, R₆, R₇, R₈, R₉ and R₁₀ are as originally defined, can be prepared by the method shown in Scheme 11, step p. The compound of Formula 11.2, wherein R₄, R₅, R₆ and R₇ are as previously defined, can be treated with an amide, such as a compound of Formula 9.1, wherein R₈, R₉ and R₁₀ are as originally defined, in the presence of a dehydrating reagent, such as oxalyl chloride ((COCl)₂) or phosphoryl trichloride (POCl₃), in a solvent, such as DCM or toluene, at a temperature from about ambient temperature to about reflux (˜40° C. or ˜111° C., respectively), to afford compounds of Formula 11.3, wherein R₄, R₅, R₆, R₇, R₈, R₉ and R₁₀ are as originally defined, as shown in step p. Compounds of Formula 11.5, wherein R₁, R₂ and R₃ are as originally defined, can be prepared by the method shown in Scheme 11, step q. The compound of Formula 11.4, wherein R₁, R₂ and R₃ are as originally defined, can be treated with an amine, such as N,O-dimethylhydroxylamine, in the presence of a base, such as pyridine, in a solvent, such as DCM, at a temperature from about 0° C. to about ambient temperature, to afford compounds of Formula 11.5, wherein R₁, R₂ and R₃ are as originally defined, as shown in step q. Compounds of Formula 9.2, wherein R₁, R₂, R₃, R₄, R₅, R₆, R₇, R₈, R₉ and R₁₀ are as originally defined, can be prepared by the method shown in Scheme 11, step r. The compound of Formula 11.3, wherein R₄, R₅, R₆, R₇, R₈, R₉ and R₁₀ are as originally defined, can be treated with a base, such as n-butyllithium, in the presence of an amide, such as a compound of Formula 11.5, wherein R₁, R₂ and R₃ are as originally defined, in a solvent, such as THF, at a temperature from about −78° C. to about 40° C., to afford compounds of Formula 9.2, wherein R₁, R₂, R₃, R₄, R₅, R₆, R₇, R₈, R₉ and R₁₀ are as originally defined, as shown in step r.

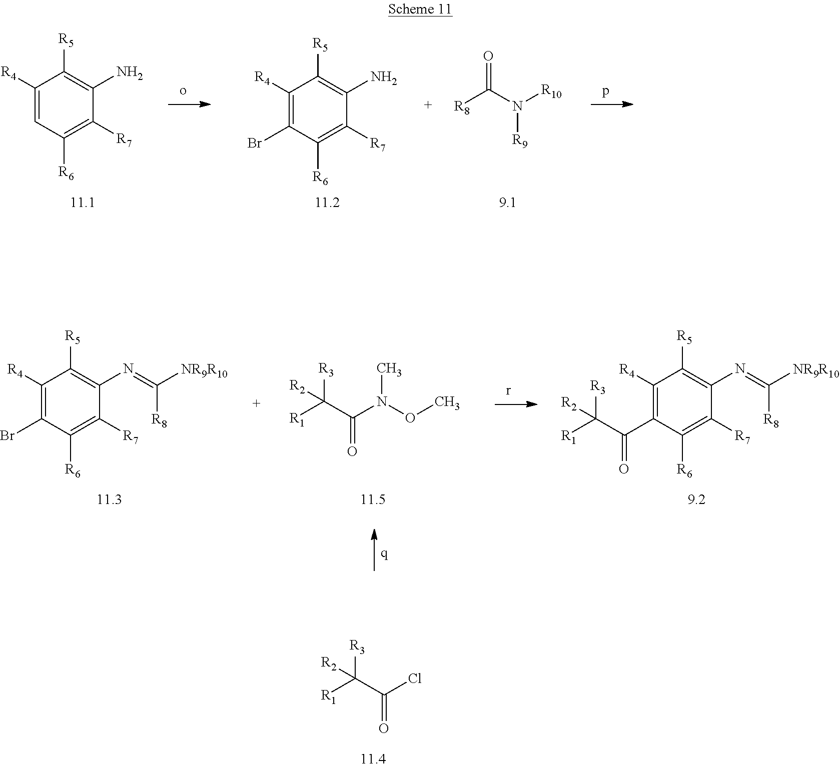

Alternatively, compounds of Formula 9.2, wherein R₁, R₂, R₃, R₄, R₅, R₆, R₇, R₈, R₉ and R₁₀ are as originally defined, can be prepared by the method shown in Scheme 12, steps s-t. Compounds of Formula 12.2, wherein R₁, R₂, R₃, R₄, R₅, R₆, R₇, R₈, R₉ and R₁₀ are as originally defined, can be prepared by the method shown in Scheme 12, step s. The compound of Formula 11.3, wherein R₄, R₅, R₆, R₇, R₈, R₉ and R₁₀ are as previously defined, can be treated with a metal, such as magnesium (Mg(O)), in the presence of an activator, such as 1,2-dibromoethane, and a catalyst, such as trimethylsilyl chloride, in a solvent, such as THF, at a temperature of about ambient temperature, to afford compounds of Formula 12.2, wherein R₁, R₂, R₃, R₄, R₅, R₆, R₇, R₈, R₉ and R₁₀ are as previously defined, as shown in step s. Compounds of Formula 9.2, wherein R₁, R₂, R₃, R₄, R₅, R₆, R₇, R₈, R₉ and R₁₀ are as originally defined, can be prepared by the method shown in Scheme 12, step t. The compound of Formula 12.2, wherein R₁, R₂, R₃, R₄, R₅, R₆, R₇, R₈, R₉ and R₁₀ are as originally defined, can be treated with an oxidant, such as pyridinium chlorochromate (PCC), in a solvent, such as DCM, at a temperature of about ambient temperature, to afford compounds of Formula 9.2, wherein R₁, R₂, R₃, R₄, R₅, R₆, R₇, R₈, R₉ and R₁₀ are as originally defined, as shown in step t.

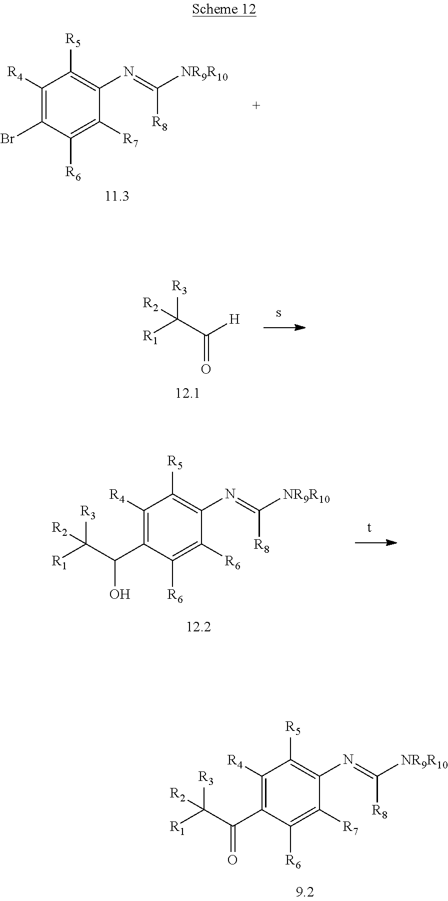

The following examples are for illustration purposes and are not to be construed as limiting this disclosure to only the embodiments disclosed in these examples.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Examples using “room temperature” or “ambient temperature” were conducted in climate controlled laboratories with temperatures ranging from about 20° C. to about 24° C. Molecules are given their known names, named according to the naming program within ChemDraw (version 17.1.0.105 (19)). If such a program is unable to name a molecule, such molecule is named using conventional naming rules. ¹H NMR spectral data are in ppm (S) and were recorded at 400 or 500 MHz; ¹³C NMR spectral data are in ppm (S) and were recorded at 101 or 126 MHz, and ¹⁹F NMR spectral data are in ppm (δ) and were recorded at 376 or 471 MHz, unless otherwise stated.

### EXAMPLES

### Example 1: Preparation of methyl (2,5-dimethylphenyl)carbamate

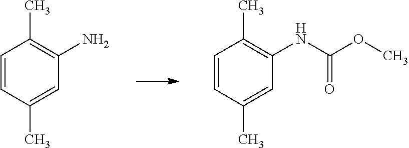

To a stirred solution of 2,5-dimethylaniline (20.0 g, 165 mmol) in acetone (180 mL) were added potassium carbonate (K₂CO₃, 68.4 grams (g), 496 millimoles (mmol)) and methyl chloroformate (38.8 milliliters (mL), 496 mmol) at room temperature. The reaction mixture was stirred at 50° C. for 16 hours (h), cooled to room temperature, and filtered. The filtrate was concentrated under reduced pressure. The obtained material was washed with n-pentane (100 mL) and diethyl ether (100 mL) to afford the title compound (23.0 g, 78% yield) as an off-white solid: ¹H NMR (300 MHz, DMSO-d₆) δ 8.75 (s, 1H), 7.15 (s, 1H), 7.05 (d, J=7.8 Hz, 1H), 6.86 (d, J=7.8 Hz, 1H), 3.63 (s, 3H), 2.23 (s, 3H), 2.13 (s, 3H); ESIMS m/z 180 ([M+H]⁺).

### Example 2A: Preparation of methyl (2,5-dimethyl-4-(2-(o-tolyl)acetyl)phenyl)carbamate

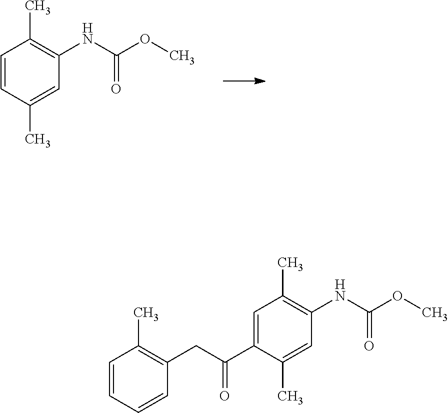 [SMILES: COC(=O)Nc1cc(C)ccc1C ; COC(=O)Nc1cc(C)c(C(=O)Cc2ccccc2C)cc1C]

A solution of 2-(o-tolyl)acetyl chloride (1.90 mL, 12.4 mmol) and zinc(II) chloride (ZnCl₂, 2.30 g, 16.9 mmol) in 1,2-dichloroethane (DCE, 20 mL) was prepared and heated to 60° C. To this solution was added a solution of methyl (2,5-dimethylphenyl)carbamate (1.50 g, 8.38 mmol) in DCE (5 mL), and the reaction mixture was heated to 90° C. and stirred for 3 h. The reaction mixture was cooled to room temperature, diluted with dichloromethane (DCM, 100 mL) and washed with water (100 mL), saturated aqueous sodium bicarbonate (NaHCO₃, 100 mL), 1 normal (N) hydrochloric acid (HCl, 50 mL), and brine (100 mL), respectively. The organic layer was dried over anhydrous sodium sulfate (Na₂SO₄), filtered, and concentrated under reduced pressure. The compound was purified by flash column chromatography (silica gel (SiO₂), 0→20% ethyl acetate in petroleum ether) to afford the title compound (0.70 g, 38% yield) as an off-white solid: mp 89-93° C.; ¹H NMR (300 MHz, DMSO-d₆) δ 9.02 (s, 1H), 7.83 (s, 1H), 7.41 (s, 1H), 7.25-7.00 (m, 4H), 4.30 (s, 2H), 3.68 (s, 3H), 2.32 (s, 3H), 2.25 (s, 3H), 2.16 (s, 3H); ¹³C NMR (75 MHz, DMSO-d₆) δ 200.13, 154.44, 139.23, 136.77, 135.64, 134.40, 133.13, 131.49, 130.60, 129.79, 127.31, 126.64, 125.77, 125.66, 51.80, 45.64, 20.66, 19.24, 17.30; ESIMS m/z 312 ([M+H]⁺).

### Example 2B: Preparation of methyl (2,5-dimethyl-4-(2-(p-tolyl)acetyl)phenyl)carbamate

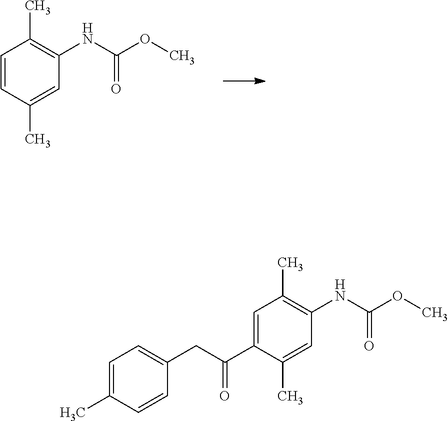

To a solution of 2-(p-tolyl)acetic acid (1.80 g, 12.0 mmol) in DCM (15 mL) were added N,N-dimethylformamide (DMF, catalytic amount (cat)) and oxalyl chloride (1.23 mL, 14.3 mmol) dropwise at 0° C., and the reaction mixture was stirred at room temperature for 4 h. The reaction mixture was concentrated under reduced pressure, and the resultant acid chloride residue was diluted with DCE (15 mL). ZnCl₂ (2.30 g, 16.7 mmol) and methyl (2,5-dimethylphenyl)carbamate (1.50 g, 8.37 mmol) in DCE (5 mL) were added, respectively, at room temperature. The reaction mixture was stirred at 60° C. for 7 h. The reaction mixture was then quenched with water (100 mL) and extracted with ethyl acetate (2×100 mL). The combined organic layer was washed with saturated aqueous NaHCO₃ (100 mL), 1 N HCl (50 mL), and brine (100 mL), respectively. The organic layer was dried over anhydrous Na₂SO₄, filtered, and concentrated under reduced pressure. The resulting material was purified by flash column chromatography (SiO₂, 0+25% ethyl acetate in petroleum ether) to afford the title compound (1.20 g, 46% yield) as a pale brown solid: mp 91-94° C.; ¹H NMR (400 MHz, DMSO-d₆) δ 9.01 (s, 1H), 7.80 (s, 1H), 7.38 (s, 1H), 7.16-7.03 (m, 4H), 4.20 (s, 2H), 3.67 (s, 3H), 2.30 (s, 3H), 2.25 (s, 3H), 2.23 (s, 3H); ESIMS m/z 312 ([M+H]⁺).

### Example 2C: Preparation of methyl (2,5-dimethyl-4-(2-(3-(trifluoromethyl)phenyl) acetyl)phenyl)carbamate

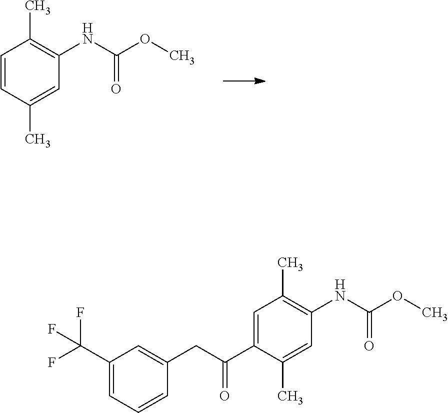 [SMILES: COC(=O)Nc1cc(C)ccc1C ; COC(=O)Nc1cc(C)c(C(=O)Cc2cccc(C(F)(F)F)c2)cc1C]

To a solution of 2-(3-(trifluoromethyl)phenyl)acetic acid (1.90 g, 11.4 mmol) in DCM (15 mL) was added thionyl chloride (1.72 mL, 23.7 mmol) dropwise at 0° C., and the reaction mixture was stirred at reflux for 2 h. The reaction mixture was concentrated under reduced pressure, and the resultant acid chloride residue was diluted with DCE (15 mL). ZnCl₂ (1.52 g, 11.2 mmol) and methyl (2,5-dimethylphenyl)carbamate (1.00 g, 5.59 mmol) were added at room temperature. The reaction mixture was then stirred at 60° C. for 7 h. The reaction mixture was quenched with water (100 mL) and extracted with ethyl acetate (2×100 mL). The organic layer was washed sequentially with saturated aqueous NaHCO₃ (100 mL), 1 N HCl (50 mL), and brine (100 mL). The organic layer was dried over anhydrous Na₂SO₄, filtered, and concentrated under reduced pressure. The resulting material was purified by flash column chromatography (SiO₂, 0→20% ethyl acetate in petroleum ether) to afford the title compound (1.20 g, 59% yield) as an off-white solid: mp 110-113° C.; ¹H NMR (400 MHz, DMSO-d₆) δ 9.05 (s, 1H), 7.87 (s, 1H), 7.70-7.50 (m, 4H), 7.43 (s, 1H), 4.45 (s, 2H), 3.68 (s, 3H), 2.34 (s, 3H), 2.26 (s, 3H); ¹³C NMR (101 MHz, DMSO-d₆) δ 199.35, 154.41, 139.51, 136.95, 136.13, 134.16, 132.43, 131.91, 129.06, 128.66, 127.23, 126.50, 125.70, 123.12, 123.08, 51.82, 46.41, 20.93, 17.30; ¹⁹F NMR (376 MHz, DMSO-d₆) δ −60.99; ESIMS m z 366 ([M+H]⁺).

### Example 2D: Preparation of methyl (4-(2-cyclohexylacetyl)-2,5-dimethylphenyl)carbamate

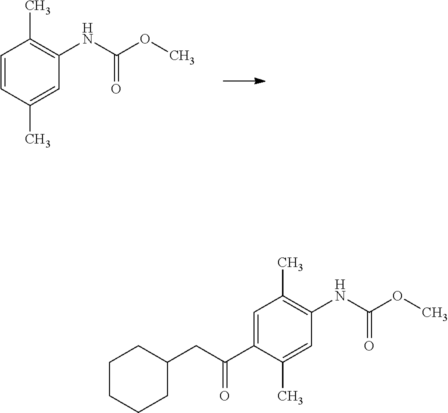 [SMILES: COC(=O)Nc1cc(C)ccc1C ; COC(=O)Nc1cc(C)c(C(=O)CC2CCCCC2)cc1C]

Aluminum(III) chloride (AlCl₃, 1.12 g, 8.37 mmol) and methyl (2,5-dimethylphenyl)carbamate (0.500 g, 2.79 mmol) were added to a round bottom flask and dissolved with DCM (15.0 mL) at room temperature. To this reaction mixture was added 2-cyclohexylacetyl chloride (0.856 mL, 5.58 mmol) dropwise. The reaction mixture was stirred at 60° C. for 1 h. The reaction mixture was quenched by pouring into ice cold water (200 mL) and was extracted with DCM (3×100 mL). The organic layer was dried over anhydrous Na₂SO₄, filtered, and concentrated under reduced pressure. The resulting material was purified by flash column chromatography (SiO₂, 0→100% ethyl acetate in hexanes) to afford the title compound (0.846 g, quant.) as a white solid: mp 110-113° C.; ¹H NMR (400 MHz, DMSO-d₆) δ 8.99 (s, 1H), 7.60 (s, 1H), 7.38 (s, 1H), 3.67 (s, 3H), 2.76 (d, J=6.8 Hz, 2H), 2.34 (s, 3H), 2.22 (s, 3H), 1.71-1.54 (m, 6H), 1.32-1.05 (m, 3H), 1.04-0.87 (m, 2H); ¹³C NMR (101 MHz, DMSO-d₆) δ 203.27, 154.95, 139.50, 135.77, 134.32, 131.75, 127.88, 126.34, 52.29, 48.63, 34.56, 33.07, 26.31, 26.15, 21.18, 17.75; ESIMS m/z 304 ([M+H]⁺).

### Example 2E: Preparation of methyl (2,5-dimethyl-4-(2-(2,4,6-trifluorophenyl)acetyl)phenyl)carbamate

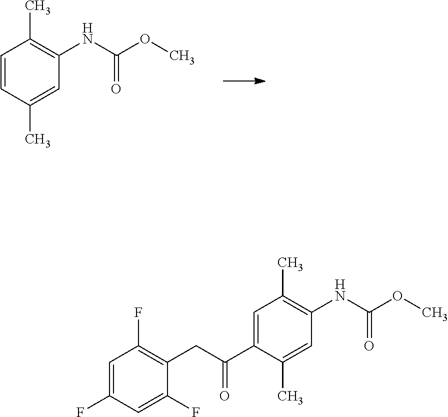 [SMILES: COC(=O)Nc1cc(C)ccc1C ; COC(=O)Nc1cc(C)c(C(=O)Cc2c(F)cc(F)cc2F)cc1C]

To the solution of 2-(2,4,6-trifluorophenyl)acetic acid (0.796 g, 4.18 mmol) in DCM (15 mL) were added DMF (cat) and oxalyl chloride (2.36 mL, 27.9 mmol) dropwise, and the reaction mixture was stirred at room temperature for 4 h. The reaction mixture was concentrated under reduced pressure, and the resultant acid chloride residue was diluted with DCM (15 mL). AlCl₃ (1.12 g, 8.37 mmol) and methyl (2,5-dimethylphenyl)carbamate (0.500 g, 2.79 mmol) were added at room temperature. The reaction mixture was stirred at 40° C. for 0.5 h. The reaction mixture was quenched with water (50 mL) and extracted with ethyl acetate (2×50 mL). The organic layer was dried over anhydrous magnesium sulfate (MgSO₄), filtered, and concentrated under reduced pressure. The resulting material was purified by flash column chromatography (SiO₂, 0→25% ethyl acetate in hexanes) to afford the title compound (0.894 g, 91% yield) as a white solid: ¹H NMR (500 MHz, CDCl₃) δ 7.89 (s, 1H), 7.64 (s, 1H), 6.73-6.65 (m, 2H), 6.57 (s, 1H), 4.22 (s, 2H), 3.81 (s, 3H), 2.51 (s, 3H), 2.28 (s, 3H); ¹⁹F NMR (471 MHz, CDCl₃) δ −109.63 (ddd, J=14.7, 9.1, 5.8 Hz), −111.73 (p, J=7.1 Hz); ESIMS m/z 352 ([M+H]⁺).

### Example 2F: Preparation of 3-(2-4-((methoxycarbonyl)amino-)2,5-dimethylphenyl)-2-oxoethyl)benzoic acid

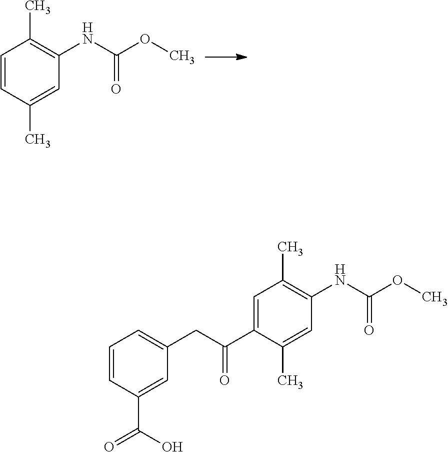 [SMILES: COC(=O)Nc1cc(C)ccc1C ; COC(=O)Nc1cc(C)c(C(=O)Cc2cccc(C(=O)O)c2)cc1C]

AlC₃ (1.12 g, 8.37 mmol) and methyl (2,5-dimethylphenyl)carbamate (0.500 g, 2.79 mmol) were added to a round bottom flask and dissolved with DCM (15 mL) at room temperature. To this reaction mixture was added 2-(3-(trifluoromethyl)phenyl)acetyl chloride (0.621 mL, 2.79 mmol). The reaction mixture was heated to 40° C. for 0.5 h. The reaction mixture was quenched by pouring into water (50 mL) and was extracted with ethyl acetate (2×50 mL). The organic layer was dried over anhydrous MgSO₄, filtered, and concentrated under reduced pressure. The compound was purified by flash column chromatography (SiO₂, 0→70% ethyl acetate in hexanes) to afford the title compound (0.909 g, 95% yield) as a tan foam: ¹H NMR (500 MHz, CDCl₃) δ 11.01 (s, 1H), 7.77 (s, 1H), 7.71 (d, J=1.9 Hz, 1H), 7.66 (dt, J=7.7, 1.5 Hz, 1H), 7.50 (dt, J=7.7, 1.5 Hz, 1H), 7.41 (t, J=7.7 Hz, 1H), 7.13 (s, 1H), 6.94-6.81 (m, 1H), 3.79 (s, 3H), 3.69 (s, 2H), 2.30 (s, 3H), 2.19 (s, 3H); ¹³C NMR (126 MHz, CDCl₃) δ 197.65, 176.43, 154.11, 138.63, 137.96, 136.89, 133.81, 133.71, 133.49, 131.60, 131.00, 129.21, 128.71, 123.29, 122.63, 52.63, 40.60, 20.25, 17.03; ESIMS m/z 342 ([M+H]⁺).

### Example 2G: Preparation of 2-fluoro-3-(2-(4-((methoxycarbonyl)amino)-2,5-dimethylphenyl)-2-oxoethyl)benzoic acid

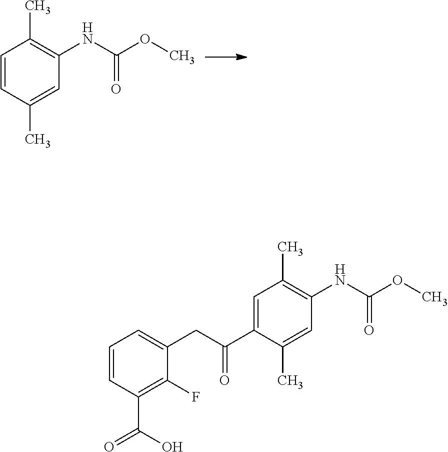 [SMILES: COC(=O)Nc1cc(C)ccc1C ; COC(=O)Nc1cc(C)c(C(=O)Cc2cccc(C(=O)O)c2F)cc1C]

To the solution of 2-(2-fluoro-3-(trifluoromethyl)phenyl))acetic acid (0.930 g, 4.18 mmol) in DCM (15 mL) were added DMF (cat) and oxalyl chloride (2.36 mL, 27.9 mmol) dropwise. The reaction mixture was stirred at room temperature for 4 h. The reaction mixture was concentrated under reduced pressure, and the resultant acid chloride residue was diluted with DCM (15 mL). AlCl₃ (1.12 g, 8.37 mmol) and methyl (2,5-dimethylphenyl)carbamate (0.500 g, 2.79 mmol) were added at room temperature. The reaction mixture was stirred at 40° C. for 0.5 h. The reaction mixture was quenched with water (50 mL) and was extracted with ethyl acetate (2×50 mL). The organic layer was dried over anhydrous MgSO₄, filtered, and concentrated under reduced pressure. The compound was purified by flash column chromatography (SiO₂, 0→₇₅% ethyl acetate in hexanes) to afford the title compound (0.291 g, 29% yield) as a pale brown solid: ¹H NMR (500 MHz, CDCl₃) δ 7.89 (s, 1H), 7.50 (ddd, J=8.2, 6.7, 1.8 Hz, 1H), 7.42 (td, J=7.2, 1.8 Hz, 1H), 7.24-7.18 (m, 2H), 6.68 (s, 1H), 3.80 (s, 3H), 3.73 (d, J=1.4 Hz, 2H), 2.49 (s, 3H), 2.13 (s, 3H) (no COOH); ¹⁹F NMR (471 MHz, CDCl₃) δ-115.33 (t, J=6.9 Hz); ESIMS m/z 360 ([M+H]⁺).

### Example 3A: Preparation of 1-(4-amino-2,5-dimethylphenyl)-2-(o-tolyl)ethan-1-one

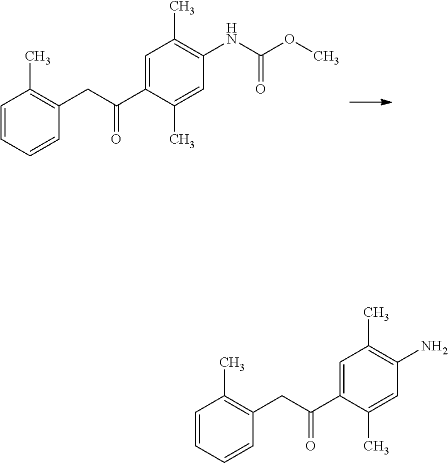 [SMILES: COC(=O)Nc1cc(C)c(C(=O)Cc2ccccc2C)cc1C ; Cc1cc(C(=O)Cc2ccccc2C)c(C)cc1N]

To a stirred solution of methyl (2,5-dimethyl-4-(2-(o-tolyl)acetyl)phenyl)carbamate (0.600 g, 1.85 mmol) in ethanol (15 mL) was added a solution of sodium hydroxide (NaOH, 0.369 g, 9.22 mmol) in water (1.5 mL) at room temperature, and the reaction mixture was stirred at reflux for 7 h. The reaction mixture was concentrated under reduced pressure. The obtained residue was purified by flash column chromatography (neutral alumina, 0→20% ethyl acetate in petroleum ether) to afford the title compound (0.330 g, 70% yield) as an off-white solid: mp 100-104° C.; ¹H NMR (300 MHz, DMSO-d₆) δ 7.70 (s, 1H), 7.24-7.00 (m, 4H), 6.42 (s, 1H), 5.64 (s, 2H), 4.19 (s, 2H), 2.31 (s, 3H), 2.14 (s, 3H), 2.08 (s, 3H); ¹³C NMR (75 MHz, DMSO-d₆) δ 197.06, 150.38, 138.57, 136.70, 135.61, 133.31, 130.41, 129.64, 126.26, 125.51, 124.01, 117.08, 116.27, 44.35, 22.07, 19.31, 16.92; ESIMS m/z 254 ([M+H]⁺).

### Example 3B: Preparation of methyl 3-(2-(4-amino-2,5-dimethylphenyl)-2-oxoethyl)benzoate

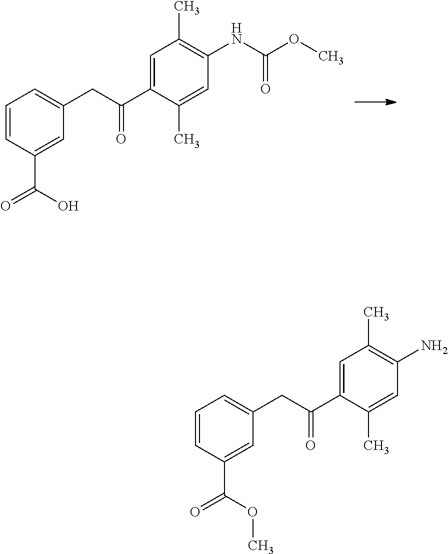 [SMILES: COC(=O)Nc1cc(C)c(C(=O)Cc2cccc(C(=O)O)c2)cc1C ; COC(=O)c1cccc(CC(=O)c2cc(C)c(N)cc2C)c1]

To a stirred solution of 3-(2-4-((methoxycarbonyl)amino-)2,5-dimethylphenyl)-2-oxoethyl)benzoic acid (0.909 g, 2.66 mmol) in tetrahydrofuran (THF, 13 mL) and methanol (MeOH, 13 mL) was added a 1 molar (M) solution of NaOH (13 mL) at room temperature, and the reaction mixture was stirred at 60° C. for 22 h. The reaction mixture was partitioned between 1 M HCl (50 mL) and ethyl acetate. The organic layer was dried over anhydrous MgSO₄, filtered, and concentrated under reduced pressure to give a yellow oil. The oil was dissolved in fresh MeOH (13 mL). A 2 M solution of trimethylsilyldiazomethane in hexanes (1.33 mL, 2.66 mmol) was added. The reaction mixture was stirred at room temperature for 1.5 h and was concentrated under reduced pressure to afford the title compound (0.645 g, 82% yield) as a brown oil: ¹H NMR (500 MHz, CDCl₃) δ 7.56 (s, 1H), 7.52 (d, J=7.7 Hz, 1H), 7.35 (d, J=7.6 Hz, 1H), 7.28 (t, J=7.6 Hz, 1H), 7.02 (s, 1H), 6.42 (s, 1H), 4.02-3.93 (m, 2H), 3.58 (s, 3H), 3.56 (s, 2H), 2.25 (s, 3H), 1.97 (s, 3H); ESIMS m/z 298 ([M+H]⁺).

### Example 4A: Preparation of (E)-N′-(2,5-dimethyl-4-(2-(o-tolyl)acetyl)phenyl)-N-ethyl-N-methylformimidamide

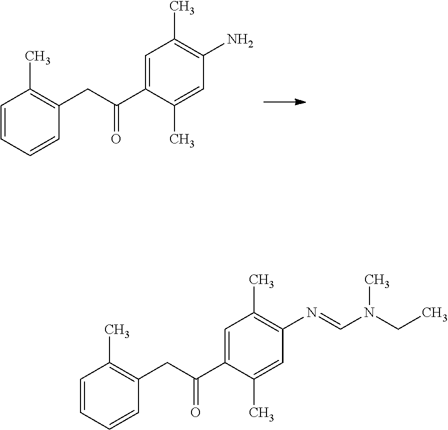 [SMILES: Cc1cc(C(=O)Cc2ccccc2C)c(C)cc1N ; CCN(C)/C=N/c1cc(C)c(C(=O)Cc2ccccc2C)cc1C]

A solution of 1-(4-amino-2,5-dimethylphenyl)-2-(o-tolyl)ethan-1-one (0.400 g, 1.58 mmol) in trimethyl orthoformate (20 mL) was stirred at 100° C. for 6 h. The reaction mixture was concentrated under reduced pressure. MeOH (15 mL), 1,4-dioxane (15 mL), and N-ethylmethylamine (0.30 mL, 3.46 mmol) were added at room temperature. The reaction mixture was stirred at 80° C. for 2 h in a sealed tube. The reaction mixture was concentrated under reduced pressure and purified by preparative high-performance liquid chromatography (HPLC) to afford the title compound (0.098 g, 19% yield) as a pale yellow solid: mp 101-105° C.; ¹H NMR (400 MHz, DMSO-d₆) δ 7.84-7.60 (m, 2H), 7.20-7.05 (m, 4H), 6.69 (br s, 1H), 4.27 (s, 2H), 3.55-3.36 (m, 2H), 3.00-2.85 (m, 3H), 2.35 (s, 3H), 2.21 (s, 3H), 2.15 (s, 3H), 1.14 (t, J=7.0 Hz, 3H); ¹³C NMR (101 MHz, DMSO-d₆) δ 199.11, 153.72, 152.67, 137.08, 136.74, 135.03, 131.65, 130.52, 129.96, 129.72, 127.73, 126.46, 125.59, 121.47, 46.88, 45.16, 31.48, 21.29, 19.28, 17.43, 14.07; ESIMS m z 323 ([M+H]⁺).

### Example 4B: Preparation of (E)-N′-(4-(2-cyclopentylacetyl)-2,5-dimethylphenyl)-N,N-dimethylformimidamide

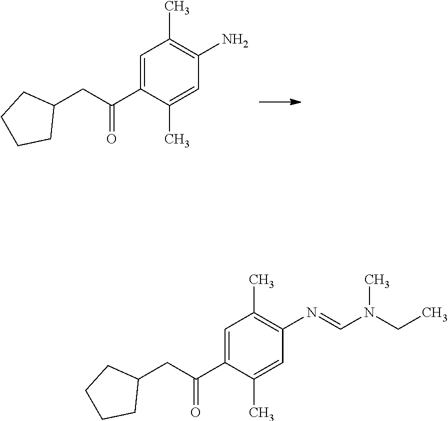 [SMILES: Cc1cc(C(=O)CC2CCCC2)c(C)cc1N ; CCN(C)/C=N/c1cc(C)c(C(=O)CC2CCCC2)cc1C]

To a solution of 1-(4-amino-2,5-dimethylphenyl)-2-cyclopentylethan-1-one (0.110 g, 0.475 mmol) in toluene (2.40 mL) was added 1,1-dimethoxy-N,N-dimethylmethanamine (126 μL, 0.951 mmol). The reaction mixture was stirred at 90° C. for 24 h and then concentrated under reduced pressure. The resulting material was purified by flash column chromatography (SiO₂, 0→5% ethyl acetate in dichloromethane) to afford the title compound (0.105 g, 77% yield) as a colorless film: ¹H NMR (500 MHz, CDCl₃) δ 7.50 (s, 1H), 7.45 (s, 1H), 6.57 (s, 1H), 3.03 (s, 6H), 2.90 (d, J=7.2 Hz, 2H), 2.47 (s, 3H), 2.37-2.29 (m, 1H), 2.27 (s, 3H), 1.85 (ddt, J=16.1, 11.9, 4.7 Hz, 2H), 1.62 (qd, J=9.7, 8.2, 3.6 Hz, 2H), 1.54 (dq, J=9.0, 4.2, 3.8 Hz, 2H), 1.21-1.11 (m, 2H); ¹³C NMR (126 MHz, CDCl₃) δ 203.17, 153.59, 152.29, 137.84, 131.69, 131.54, 128.36, 122.34, 47.19, 40.14, 36.51, 34.29, 32.71, 25.02, 21.64, 17.63; ESIMS m/z 287 ([M+H]⁺).

### Example 4C: Preparation of (E)-N-(4-2-cyclopentylacetyl)-2,5-dimethlphenyl)-N-ethyl-N-methylacetimidamide

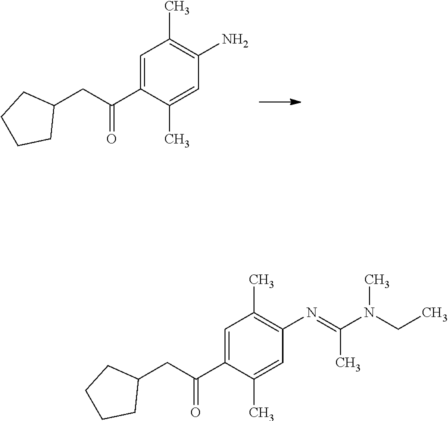 [SMILES: Cc1cc(C(=O)CC2CCCC2)c(C)cc1N ; CCN(C)/C(C)=N/c1cc(C)c(C(=O)CC2CCCC2)cc1C]

To a solution of N-ethyl-N-methylacetamide (0.131 g, 1.30 mmol) in DCE (6.50 mL) was added phosphoryl trichloride (121 μL, 1.30 mmol). The reaction mixture was stirred at room temperature for 2 h. 1-(4-Amino-2,5-dimethylphenyl)-2-cyclopentylethan-1-one (300 mg, 1.30 mmol) was added, and the reaction mixture was stirred at 80° C. for 2 h. The reaction was then quenched with 1 M NaOH (5 mL), and the reaction mixture was extracted with DCM (2×10 mL). The organic layer was washed with saturated NaHCO₃, dried through a phase separator, and concentrated under reduced pressure. The resulting material was purified by flash column chromatography (SiO₂, 0→15% ethyl acetate in dichloromethane) to afford the title compound (0.141 g, 35% yield) as a gold film: ¹H NMR (500 MHz, CDCl₃) δ 7.50 (s, 1H), 6.45 (s, 1H), 3.45 (q, J=7.1 Hz, 2H), 3.00 (s, 3H), 2.90 (d, J=7.2 Hz, 2H), 2.45 (s, 3H), 2.33 (p, J=8.2 Hz, 1H), 2.07 (s, 3H), 1.89-1.80 (m, 2H), 1.79 (s, 3H), 1.67-1.59 (m, 2H), 1.58-1.49 (m, 2H), 1.23-1.10 (m, 5H); ¹³C NMR (126 MHz, CDCl₃) δ 203.19, 155.13, 154.18, 137.61, 131.69, 130.98, 126.57, 125.34, 47.11, 44.64, 36.54, 35.32, 32.70, 25.00, 21.60, 17.73, 15.10, 12.74; ESIMS m z 315 ([M+H]⁺).

### Example 4D: Preparation of (E N′-(4-(2-cyclohexylacetyl)-methoxy-2-methylphenyl)-N ethyl-N-methylformimidamide

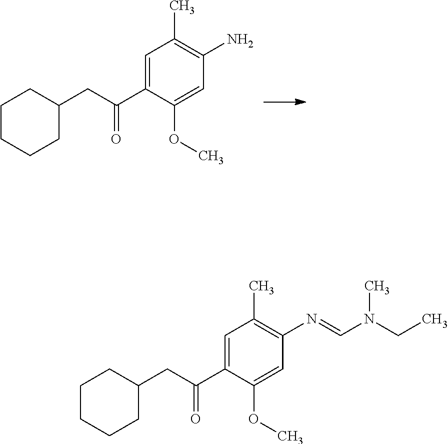 [SMILES: COc1cc(N)c(C)cc1C(=O)CC1CCCCC1 ; CCN(C)/C=N/c1cc(OC)c(C(=O)CC2CCCCC2)cc1C]

Preparation of a stock solution of Vilsmeier reagent: An oven-dried vial equipped with a stir bar was charged with N-ethyl-N-methylformamide (0.240 g, 2.30 mmol, 85% weight per weight (w/w)) and DCM (2.40 mL). The resultant yellow solution was cooled to 0° C. in an ice bath, and oxalyl chloride (0.200 mL, 2.30 mmol) was added dropwise. An immediate evolution of gas and an exotherm were observed. After the evolution of gas ceased, the ice bath was removed, and the reaction mixture was stirred at room temperature for 2 hours. The reagent became bright yellow. Reaction with substrate: 1-(4-Amino-2-methoxy-5-methylphenyl)-2-cyclohexylethan-1-one (0.190 g, 0.727 mmol) was added to a vial equipped with a stir bar, and DCM (7.60 mL) was added. A portion of the solution of the Vilsmeier reagent prepared above (2.40 mL, 2.30 mmol) was added to the solution of the aniline while stirring, and the reaction mixture was stirred overnight. The material was purified by flash column chromatography (SiO₂, 0→100% ethyl acetate-hexane gradient) to afford the title compound (0.179 g, 75% yield) as a yellow semi-solid: ¹H NMR (400 MHz, CDCl₃) δ 7.53 (d, J=0.8 Hz, 1H), 7.47 (s, 1H), 6.30 (s, 1H), 3.86 (s, 3H), 3.52 (s, 1H), 3.02 (s, 3H), 2.81 (d, J=6.8 Hz, 2H), 2.18 (s, 3H), 1.90 (dtd, J=14.7, 7.4, 3.4 Hz, 1H), 1.73 (d, J=2.5 Hz, 1H), 1.70 (s, 2H), 1.75-1.66 (m, 1H), 1.64 (s, 2H), 1.35-1.18 (m, 5H), 1.14 (dd, J=12.1, 3.5 Hz, 1H), 0.97 (qd, J=12.2, 2.8 Hz, 2H); ESIMS m/z 331 ([M+H]⁺).

### Example 4E: Preparation of (Z)—N′-(4-(2-cyclohexylacetyl)-2,5-dimethylphenyl)-N,N-dimethylcarbamimidothioic acid

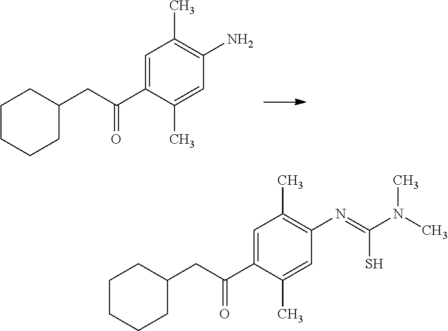 [SMILES: Cc1cc(C(=O)CC2CCCCC2)c(C)cc1N ; Cc1cc(C(=O)CC2CCCCC2)c(C)cc1/N=C(\S)N(C)C]

A solution of 1-(4-amino-2,5-dimethylphenyl)-2-cyclohexylethan-1-one (0.100 g, 0.408 mmol) and NaHCO₃ (0.342 g, 4.08 mmol) was prepared in DCM (1.24 mL) and H₂O (1.24 mL). To this solution was added thiophosgene (34.4 μL, 0.448 mmol) dropwise via syringe. The resulting orange biphasic mixture was stirred vigorously at room temperature for 2 h. The biphasic mixture was diluted with H₂O (5 mL) and DCM (5 mL) and passed through a phase separator and concentrated to afford a pale yellow oil. The resulting oil was used directly in the next step.

The material from the previous step was dissolved in DCM (1.24 mL), and dimethylamine (0.408 mL, 0.815 mmol) was added in one portion via syringe. The resulting solution was stirred at room temperature for 1 h. The reaction mixture was directly purified by flash column chromatography (SiO₂, 0+50% EtOAc in hexanes) to afford the title compound (0.105 g, 77% yield) as a white solid; ¹H NMR (400 MHz, CDCl₃) δ 7.47 (s, 1H), 7.04 (s, 1H), 6.80 (s, 1H), 3.34 (s, 6H), 2.74 (d, J=6.8 Hz, 2H), 2.44 (s, 3H), 2.28 (s, 3H), 1.95 (ddt, J=11.3, 7.4, 3.4 Hz, 1H), 1.76 (d, J=3.8 Hz, 1H), 1.70 (d, J=16.0 Hz, 4H), 1.37-1.23 (m, 2H), 1.27-1.11 (m, 1H), 1.06-0.92 (m, 2H); ESIMS m/z 333 ([M+H]⁺).

### Example 5: Preparation of 4-bromo-2-methyl-5-(trifluoromethyl)aniline

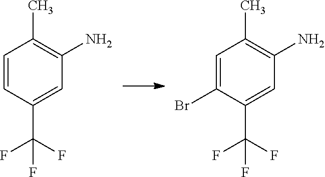

To a solution of 2-methyl-5-(trifluoromethyl)aniline (0.88 g, 5.0 mmol) in dry acetonitrile (15 mL) at 10° C., N-bromosuccinimide (NBS, 1.023 g, 5.750 mmol) was added in small portions, and the reaction mixture was stirred at the same temperature for 1 h. The reaction mixture was concentrated under reduced pressure, and the resulting product was purified by flash column chromatography (SiO₂, 0+10% dichloromethane in hexane) to afford the title compound (0.941 g, 74% yield) as a brown liquid: ¹H NMR (400 MHz, CDCl₃) δ 7.34 (s, 1H), 6.96 (s, 1H), 3.76 (s, 2H), 2.16 (s, 3H); ¹⁹F NMR (376 MHz, CDCl₃) δ −62.35; EIMS m/z 254 [M+].

### Example 6: Preparation of (E)-N′-(4-bromo-2,5-dimethylphenyl)-N-ethyl-N-methylformimidamide

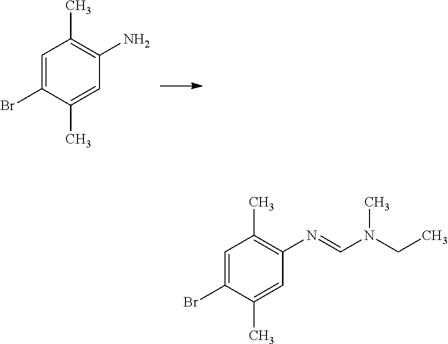

To a solution of N-ethyl-N-methylformamide (0.653 g, 7.50 mmol) in dry DCM (7 mL) at 0° C., oxalyl chloride (0.643 mL, 7.50 mmol) was added dropwise, and the reaction mixture was stirred at ambient temperature for 30 minutes. The solution was added dropwise to a solution of 4-bromo-2,5-dimethylaniline (1.00 g, 5.00 mmol) in dry DCM (5 mL), and the reaction mixture was stirred for 1.5 h at ambient temperature. A saturated aqueous solution of sodium carbonate (Na₂CO₃) was added dropwise until the pH of the reaction mixture was higher than 9, and H₂O (7 mL) and DCM (7 mL) were added. The organic phase was separated, and the solvent was removed under reduced pressure. The resulting material was purified by flash column chromatography (SiO₂, 0>20% ethyl acetate in hexane) to afford the title compound (0.967 g, 72% yield) as a brown solid: ¹H NMR (500 MHz, CDCl₃) δ 7.39 (s, 1H), 7.26 (s, 1H), 6.60 (s, 1H), 3.34-3.31 (m, 2H), 2.98 (s, 3H), 2.31 (s, 3H), 2.20 (s, 3H), 1.20 (t, J=7.2 Hz, 3H); ¹³C NMR (126 MHz, CDCl₃) δ 151.86, 150.45, 135.29, 133.26, 131.10, 121.41, 117.26, 47.86, 32.05, 21.91, 16.75, 14.35; ESIMS m/z 269 ([M+H]⁺).

### Example 7: Preparation of 2-cyclohexyl-N-methoxy-N-methylacetamide

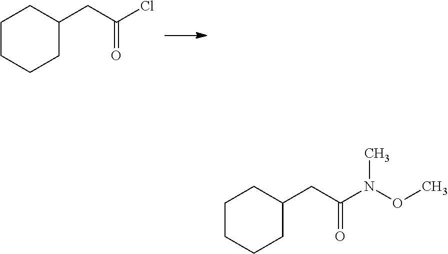

A solution of N,O-dimethylhydroxylamine (0.761 g, 12.5 mmol) and 2-cyclohexylacetyl chloride (0.711 mL, 6.23 mmol) was prepared in in DCM (20.0 mL) and cooled to 0° C. in an ice/water bath under an atmosphere of argon. Pyridine (1.10 mL, 13.7 mmol) was added dropwise via syringe. The mixture was stirred at 0° C. for 5 minutes and was allowed to warm to room temperature. A precipitate formed upon warming. The reaction mixture was allowed to stir at room temperature overnight, and the reaction was quenched by washing subsequently with 1 M HCl (2×20 mL), saturated NaHCO₃ (2×20 mL) and brine (1×20 mL). The organic layer was dried over Na₂SO₄ and concentrated under reduced pressure to afford the title compound (0.750 g, 65% yield) as a clear, colorless oil: ¹H NMR (400 MHz, CDCl₃) δ 3.67 (s, 3H), 3.18 (s, 3H), 2.30 (d, J=7.0 Hz, 2H), 1.85 (tdp, J=10.6, 7.0, 3.4 Hz, 1H), 1.79-1.60 (m, 4H), 1.29 (dtd, J=13.1, 9.5, 3.4 Hz, 2H), 1.25-1.07 (m, 2H), 1.04-0.89 (m, 2H); ESIMS m/z 186 ([M+H]⁺).

### Example 8: Preparation of (E)-N′-(4-(cyclobutanecarbonyl)-2,5-dimethylphenyl)-N-ethyl-N-methylformimidamide

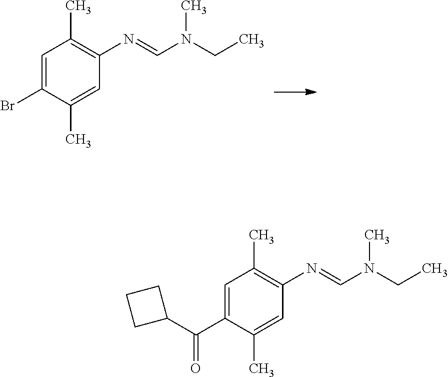 [SMILES: CCN(C)/C=N/c1cc(C)c(Br)cc1C ; CCN(C)/C=N/c1cc(C)c(C(=O)C2CCC2)cc1C]

To a solution of (E)-N′-(4-bromo-2,5-dimethylphenyl)-N-ethyl-N-methylformimidamide (0.162 g, 0.600 mmol) in dry THF (4.00 mL) under nitrogen, n-butyllithium (0.260 mL, 0.660 mmol, 2.5 M in Hexanes) was added dropwise at −78° C., and the solution was stirred at the same temperature for 30 minutes. A solution of N-methoxy-N-methylcyclobutanecarboxamide (95 mg, 0.66 mmol) in dry THE (2 mL) was added dropwise, and the resulting mixture was stirred at −78° C. for 15 minutes. The mixture was allowed to warm to ambient temperature and was stirred for 1 h. The reaction was quenched with aqueous 1 M HCl (3 mL). The solution was stirred for 5 min, and a saturated aqueous solution of Na₂CO₃ was added dropwise until the pH of the solution was higher than 9. DCM was added, and the phases were separated. The combined organic layers were passed through a phase separator and concentrated. The resulting material was purified by flash column chromatography (SiO₂, 0→5% EtOH in hexane) to afford the title compound (0.060 g, 36% yield) as a yellow oil: ¹H NMR (500 MHz, CDCl₃) δ 7.49 (s, 1H), 7.41 (s, 1H), 6.57 (s, 1H), 3.94 (pd, J=8.6, 1.1 Hz, 1H), 3.58-3.25 (m, 2H), 3.00 (s, 3H), 2.52 (s, 3H), 2.42-2.32 (m, 2H), 2.27-2.18 (m, 5H), 2.02 (dp, J=11.0, 8.8 Hz, 1H), 1.90-1.81 (m, 1H), 1.21 (t, J=7.2 Hz, 3H); ¹³C NMR (126 MHz, CDCl₃) δ 203.30, 153.94, 151.77, 138.73, 131.82, 129.45, 128.35, 122.41, 47.80, 43.75, 31.98, 25.42, 21.86, 17.94, 17.67, 14.33; ESIMS m/z 273 ([M+H]⁺).

### Example 9: Preparation of (E)-N-ethyl-N′-(4-(1-hydroxy-3,3-dimethylbutyl)-2,3,5-trimethylphenyl)-N-methylformimidamide

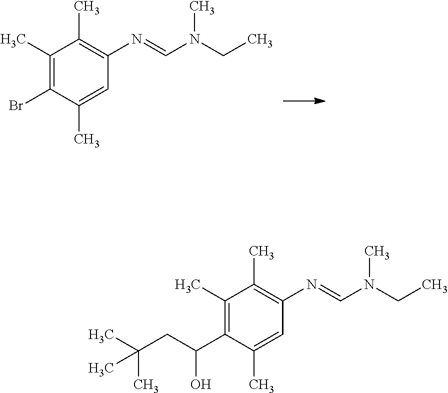 [SMILES: CCN(C)/C=N/c1cc(C)c(Br)c(C)c1C ; CCN(C)/C=N/c1cc(C)c(C(O)CC(C)(C)C)c(C)c1C]

Magnesium metal turnings (0.0386 g, 1.59 mmol) were added to a 25 mL vial, and the vial was evacuated and backfilled with nitrogen gas (3×). THF (1.33 mL), 1,2-dibromoethane (4.97 mg, 0.0260 mmol), and chlorotrimethylsilane (0.00575 g, 0.0530 mmol) were added sequentially. The mixture was stirred at room temperature for 10 minutes. A solution of (E)-N′-(4-bromo-2,3,5-trimethylphenyl)-N-ethyl-N-methylformimidamide (0.150 g, 0.530 mmol) in THF (1.00 mL) was added dropwise, and the reaction mixture was stirred at room temperature for 2 h. A solution of 3,3-dimethylbutanal (0.080 g, 0.794 mmol) in THF (1.00 mL) was added dropwise, and the reaction mixture was stirred at room temperature for 2 h. The reaction was quenched with MeOH (2 mL), and the resulting material was purified by flash column chromatography (SiO₂, 0→5% MeOH in DCM) to afford the title compound (0.035 g, 22% yield) as a clear, colorless oil: ¹H NMR (500 MHz, CDCl₃) δ 7.34 (s, 1H), 6.33 (s, 1H), 5.27 (dd, J=9.2, 2.6 Hz, 1H), 3.38 (d, J=48.0 Hz, 3H), 2.98 (d, J=1.6 Hz, 3H), 2.34 (s, 6H), 2.15 (d, J=7.8 Hz, 3H), 2.07 (dd, J=14.7, 9.1 Hz, 1H), 1.49 (dd, J=14.7, 2.6 Hz, 1H), 1.19 (t, J=7.1 Hz, 3H), 1.02 (s, 9H); ESIMS m/z 305 ([M+H]⁺).

### Example 10: Preparation of (E)-N′-(4-(3,3-dimethylbutanoyl)-2,3,5-trimethylphenyl)-N-ethyl-N-methylformimidamide

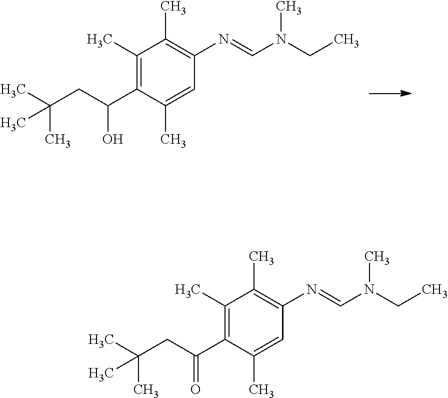

To a solution of (E)-N-ethyl-N-(4-(1-hydroxy-3,3-dimethylbutyl)-2,3,5-trimethylphenyl)-N-methylformimidamide (35.0 mg, 0.115 mmol) in DCM (0.500 mL) was added in one portion pyridinium chlorochromate (49.6 mg, 0.230 mmol), and the resulting solution was stirred at room temperature for 2 h. The reaction mixture was then filtered through a small column of Florisil© (magnesium silicate), washing thoroughly with DCM. The combined organic filtrates were concentrated under reduced pressure. The resulting material was purified by flash column chromatography (SiO₂, 0→30% EtOH in 0.5% triethylamine (Et₃N)-hexanes) to afford the title compound (0.019 g, 55% yield) as a colorless oil: ¹H NMR (500 MHz, CDCl₃) δ 7.37 (s, 1H), 6.41 (s, 1H), 3.35 (s, 2H), 2.99 (s, 3H), 2.61 (s, 2H), 2.17 (s, 3H), 2.16 (s, 3H), 2.12 (s, 3H), 1.20 (t, J=7.1 Hz, 3H), 1.12 (s, 9H); ESIMS m/z 303 ([M+H]⁺).

### General Biological Experimental Details

### Example A: Evaluation of Fungicidal Activity: Septoria Leaf Blotch of Wheat (Zymoseptoria tritici; Bayer Code SEPTTR)

Technical grades of materials were dissolved in acetone, which were then mixed with nine volumes of water (H₂O) containing 110 ppm Triton X-100. The fungicide solutions were applied onto wheat seedlings using an automated booth sprayer to run-off. All sprayed plants were allowed to air dry prior to further handling. All fungicides were evaluated using the aforementioned method for their activity vs. all target diseases, unless stated otherwise.

Wheat plants (variety ‘Yuma’) were grown from seed in a greenhouse in soil-less potting mix until the first leaf was fully emerged, with 7-10 seedlings per pot. These plants were inoculated with an aqueous spore suspension of Zymoseptoria tritici either 3 days prior to fungicide treatment (3 day curative; 3DC) or 1 day after fungicide treatment (1 day protectant; 1DP). After inoculation the plants were kept in 100% relative humidity for three days to permit spores to germinate and infect the leaf. The plants were then transferred to a greenhouse for disease to develop. When disease symptoms were fully expressed on the 1st leaves of untreated plants, infection levels were assessed on a scale of 0 to 100 percent disease severity. Percent disease control was calculated using the ratio of disease severity on treated plants relative to untreated plants.

### Example B: Evaluation of Fungicidal Activity: Wheat Brown Rust (Puccinia triticina; Synonym: Puccinia recondita f sp. tritici; Bayer Code PUCCRT)

Wheat plants (variety ‘Yuma’) were grown from seed in a greenhouse in soil-less potting mix until the first leaf was fully emerged, with 7-10 seedlings per pot. These plants were inoculated with an aqueous spore suspension of Puccinia triticina after fungicide treatments. After inoculation, the plants were kept in a dark dew room with 100% relative humidity overnight to permit spores to germinate and infect the leaf. The plants were then transferred to a greenhouse for disease to develop. Fungicide formulation, application and disease assessment followed the procedures as described in Example A.

### Example C: Evaluation of Fungicidal Activity: Asian Soybean Rust (Phakopsora pachyrhizi; Bayer Code PHAKPA)

Technical grades of materials were dissolved in acetone, which were then mixed with nine volumes of H₂O containing 0.011% Tween-20. The fungicide solutions were applied onto soybean seedlings using an automated booth sprayer to run-off. All sprayed plants were allowed to air dry prior to further handling.

Soybean plants (variety ‘Williams 82’) were grown in soil-less potting mix, with one plant per pot. Ten-day-old seedlings were used for testing. Plants were inoculated as described in Example A. Plants were incubated for 24 h in a dark dew room with 100% relative humidity then transferred to a growth room for disease to develop. Fungicide formulation and application were made as described in Example A. When disease symptoms were fully expressed, disease severity was assessed on the sprayed leaves on a scale of 0 to 100 percent. Percent disease control was calculated using the ratio of disease severity on treated plants relative to untreated plants.

### Example D: Evaluation of Fungicidal Activity: Leaf Blotch of Barley (Rhynchosporium commune; Bayer Code RHYNSE)

Barley plants (variety ‘Harrington’) were grown from seed in a greenhouse in soil-less potting mix until the first leaf was fully emerged, with 7-10 seedlings per pot. These plants were inoculated with an aqueous spore suspension of Rhynchosporium commune after fungicide treatments. After inoculation, the plants were kept in a dark dew room with 100% relative humidity for two days to permit spores to germinate and infect the leaf. The plants were then transferred to a greenhouse for disease to develop. Fungicide formulation and application were made as described in Example A. Disease assessment was conducted as described in Example A.

### Example E. Evaluation of Fungicidal Activity: Spot Blotch of Barley (Cochliobolus sativus Bayer Code COCHSA)

Barley seedlings (variety ‘Harrington’) were propagated in soil-less potting mix, with each pot having 8 to 12 plants, and used for testing when the first leaf was fully emerged. Test plants were inoculated with a spore suspension of Cochliobolus sativus 24 h after fungicide treatments. After inoculation the plants were kept in 10000 relative humidity for two days to permit spores to germinate and infect the leaf. The plants were then transferred to a greenhouse for disease to develop. Fungicide formulation, application and disease assessment followed the procedures as described in Example A.

TABLE 1
Compound Structure, Preparation Method, and Appearance

| Cmpd. | | As Prepared | |
|---|---|---|---|
| No. | Structure | According To | Appearance |
| 1 | 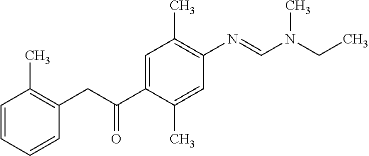 [SMILES: CCN(C)/C=N/c1cc(C)c(C(=O)Cc2ccccc2C)cc1C] | Example 1; Example 2A; Example 3A; Example 4A | Pale yellow solid |
| 2 | 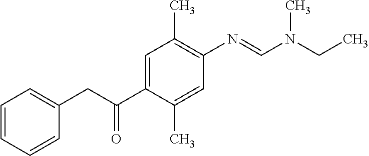 [SMILES: CCN(C)/C=N/c1cc(C)c(C(=O)Cc2ccccc2)cc1C] | Example 1; Example 2A; Example 3A; Example 4A | Pale yellow solid |
| 3 | 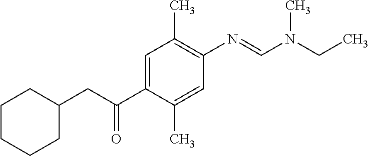 [SMILES: CCN(C)/C=N/c1cc(C)c(C(=O)CC2CCCCC2)cc1C] | Example 1; Example 2A; Example 3A; Example 4A | Off-white solid |
| 4 | 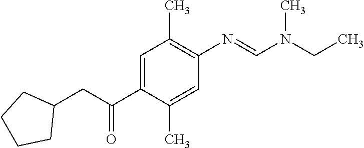 [SMILES: CCN(C)/C=N/c1cc(C)c(C(=O)CC2CCCC2)cc1C] | Example 1; Example 2A; Example 3A; Example 4A | Pale yellow liquid |
| 5 | 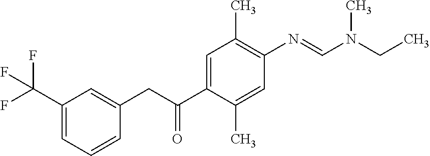 [SMILES: CCN(C)/C=N/c1cc(C)c(C(=O)Cc2cccc(C(F)(F)F)c2)cc1C] | Example 1; Example 2C; Example 3A; Example 4A | Pale yellow solid |
| 6 | 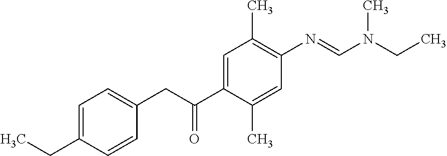 [SMILES: CCc1ccc(CC(=O)c2cc(C)c(/N=C/N(C)CC)cc2C)cc1] | Example 1; Example 2C; Example 3A; Example 4A | Yellow gummy liquid |
| 7 | 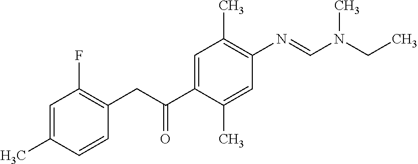 [SMILES: CCN(C)/C=N/c1cc(C)c(C(=O)Cc2ccc(C)cc2F)cc1C] | Example 1; Example 2C; Example 3A; Example 4A | Pale yellow solid |
| 8 | 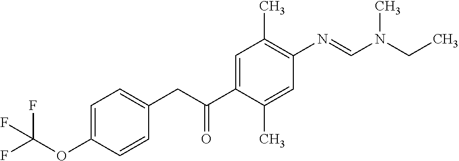 [SMILES: CCN(C)/C=N/c1cc(C)c(C(=O)Cc2ccc(OC(F)(F)F)cc2)cc1C] | Example 1; Example 2C; Example 3A; Example 4A | Off-white solid |
| 9 | 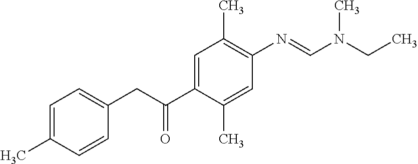 [SMILES: CCN(C)/C=N/c1cc(C)c(C(=O)Cc2ccc(C)cc2)cc1C] | Example 1; Example 2B; Example 3A; Example 4A | Yellow solid |
| 10 | 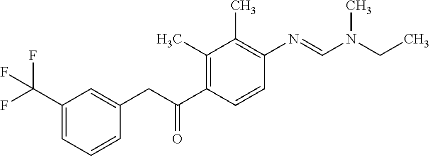 [SMILES: CCN(C)/C=N/c1ccc(C(=O)Cc2cccc(C(F)(F)F)c2)c(C)c1C] | Example 1; Example 2C; Example 3A; Example 4A | Pale yellow solid |
| 11 | 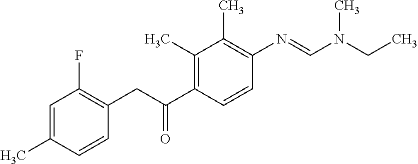 [SMILES: CCN(C)/C=N/c1ccc(C(=O)Cc2ccc(C)cc2F)c(C)c1C] | Example 1; Example 2C; Example 3A; Example 4A | Yellow liquid |
| 12 | 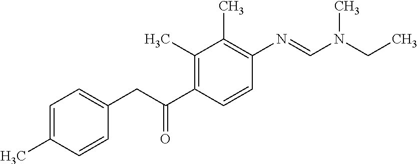 [SMILES: CCN(C)/C=N/c1ccc(C(=O)Cc2ccc(C)cc2)c(C)c1C] | Example 1; Example 2B; Example 3A; Example 4A | Pale yellow gummy liquid |
| 13 | 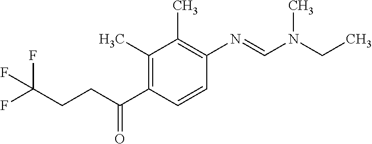 [SMILES: CCN(C)/C=N/c1ccc(C(=O)CCC(F)(F)F)c(C)c1C] | Example 1; Example 2A; Example 3A; Example 4A | Off-white solid |
| 14 | 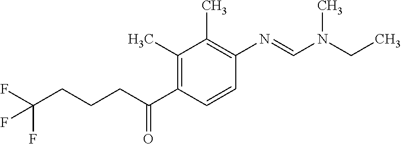 [SMILES: CCN(C)/C=N/c1ccc(C(=O)CCCC(F)(F)F)c(C)c1C] | Example 1; Example 2B; Example 3A; Example 4A | White solid |
| 15 | 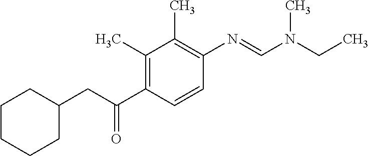 [SMILES: CCN(C)/C=N/c1ccc(C(=O)CC2CCCCC2)c(C)c1C] | Example 1; Example 2B; Example 3A; Example 4A | Off-white solid |
| 16 | 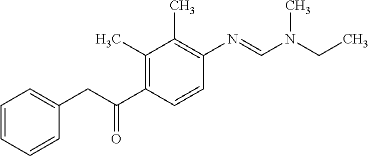 [SMILES: CCN(C)/C=N/c1ccc(C(=O)Cc2ccccc2)c(C)c1C] | Example 1; Example 2A; Example 3A; Example 4A | Pale yellow gummy liquid |
| 17 | 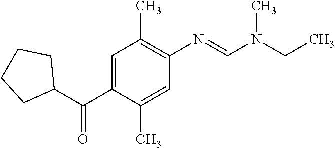 [SMILES: CCN(C)/C=N/c1cc(C)c(C(=O)C2CCCC2)cc1C] | Example 1; Example 2A; Example 3A; Example 4A | Gold oil |
| 18 | 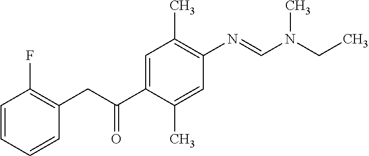 [SMILES: CCN(C)/C=N/c1cc(C)c(C(=O)Cc2ccccc2F)cc1C] | Example 1; Example 2D; Example 3A; Example 4A | Yellow crystalline solid |
| 19 | 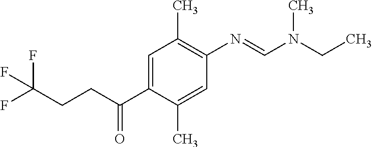 [SMILES: CCN(C)/C=N/c1cc(C)c(C(=O)CCC(F)(F)F)cc1C] | Example 1; Example 2D; Example 3A; Example 4A | White crystalline solid |
| 20 | 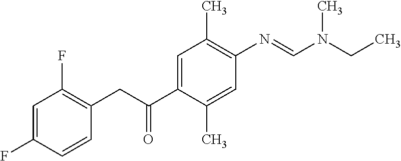 [SMILES: CCN(C)/C=N/c1cc(C)c(C(=O)Cc2ccc(F)cc2F)cc1C] | Example 1; Example 2D; Example 3A; Example 4A | Yellow foam |
| 21 | 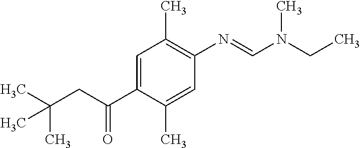 [SMILES: CCN(C)/C=N/c1cc(C)c(C(=O)CC(C)(C)C)cc1C] | Example 1; Example 2D; Example 3A; Example 4A | Yellow oil |
| 22 | 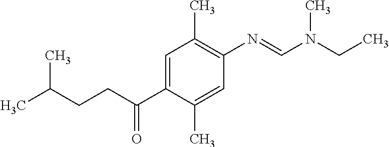 [SMILES: CCN(C)/C=N/c1cc(C)c(C(=O)CCC(C)C)cc1C] | Example 1; Example 2D; Example 3A; Example 4A | Yellow oil |
| 23 | 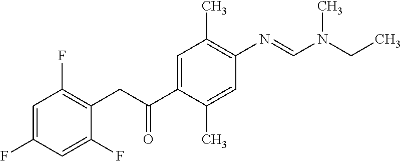 [SMILES: CCN(C)/C=N/c1cc(C)c(C(=O)Cc2c(F)cc(F)cc2F)cc1C] | Example 1; Example 2E; Example 3A; Example 4A | Beige solid |
| 24 | 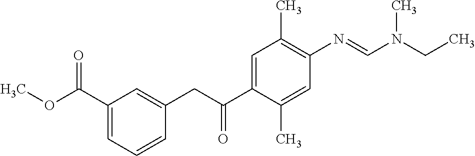 [SMILES: CCN(C)/C=N/c1cc(C)c(C(=O)Cc2cccc(C(=O)OC)c2)cc1C] | Example 1; Example 2F; Example 3B; Example 4A | Gold oil |
| 25 | 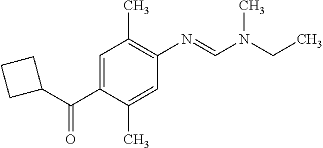 [SMILES: CCN(C)/C=N/c1cc(C)c(C(=O)C2CCC2)cc1C] | Example 1; Example 2D; Example 3A; Example 4A | Yellow oil |
| 26 | 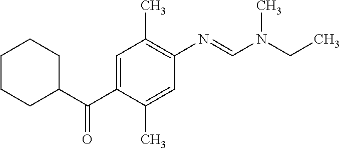 [SMILES: CCN(C)/C=N/c1cc(C)c(C(=O)C2CCCCC2)cc1C] | Example 1; Example 2D; Example 3A; Example 4A | Amber oil |
| 27 | 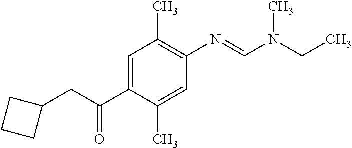 [SMILES: CCN(C)/C=N/c1cc(C)c(C(=O)CC2CCC2)cc1C] | Example 1; Example 2D; Example 3A; Example 4A | Yellow film |
| 29 | 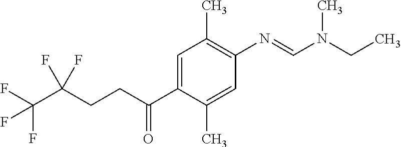 [SMILES: CCN(C)/C=N/c1cc(C)c(C(=O)CCC(F)(F)C(F)(F)F)cc1C] | Example 1; Example 2E; Example 3A; Example 4A | Yellow solid |
| 30 | 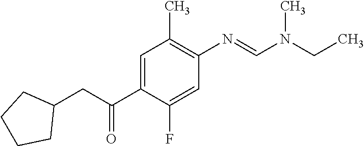 [SMILES: CCN(C)/C=N/c1cc(F)c(C(=O)CC2CCCC2)cc1C] | Example 1; Example 2D; Example 3A; Example 4A | Colorless oil |
| 31 | 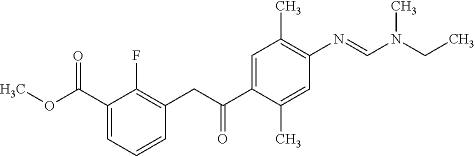 [SMILES: CCN(C)/C=N/c1cc(C)c(C(=O)Cc2cccc(C(=O)OC)c2F)cc1C] | Example 1; Example 2G; Example 3B; Example 4A | Yellow oil |
| 32 | 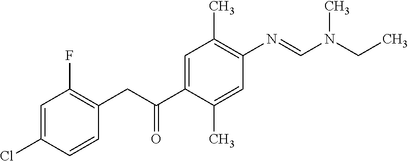 [SMILES: CCN(C)/C=N/c1cc(C)c(C(=O)Cc2ccc(Cl)cc2F)cc1C] | Example 1; Example 2E; Example 3A; Example 4A | Yellow oil |
| 33 | 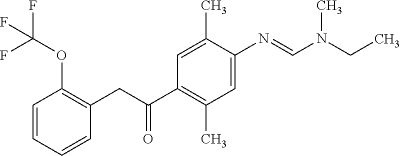 [SMILES: CCN(C)/C=N/c1cc(C)c(C(=O)Cc2ccccc2OC(F)(F)F)cc1C] | Example 1; Example 2B; Example 3A; Example 4A | Yellow oil |
| 34 | 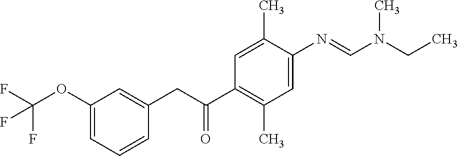 [SMILES: CCN(C)/C=N/c1cc(C)c(C(=O)Cc2cccc(OC(F)(F)F)c2)cc1C] | Example 1; Example 2B; Example 3A; Example 4A | Yellow film |
| 35 | 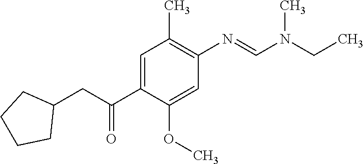 [SMILES: CCN(C)/C=N/c1cc(OC)c(C(=O)CC2CCCC2)cc1C] | Example 1; Example 2A; Example 3A; Example 4D | Colorless film |
| 36 | 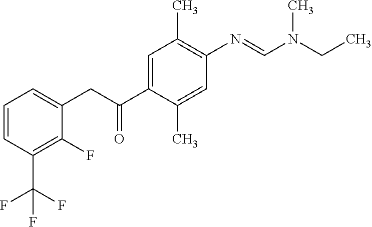 [SMILES: CCN(C)/C=N/c1cc(C)c(C(=O)Cc2cccc(C(F)(F)F)c2F)cc1C] | Example 1; Example 2B; Example 3A; Example 4D | Yellow solid |
| 37 | 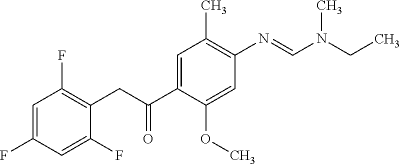 [SMILES: CCN(C)/C=N/c1cc(OC)c(C(=O)Cc2c(F)cc(F)cc2F)cc1C] | Example 1; Example 2B; Example 3A; Example 4D | White solid |
| 38 | 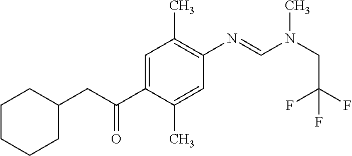 [SMILES: Cc1cc(C(=O)CC2CCCCC2)c(C)cc1/N=C/N(C)CC(F)(F)F] | Example 1; Example 2D; Example 3A; Example 4A | Yellow oil |
| 39 | 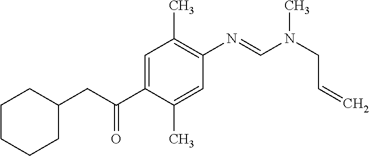 [SMILES: C=CCN(C)/C=N/c1cc(C)c(C(=O)CC2CCCCC2)cc1C] | Example 1; Example 2D; Example 3A; Example 4A | Yellow oil |
| 40 | 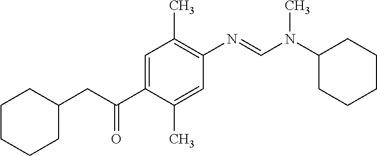 [SMILES: Cc1cc(C(=O)CC2CCCCC2)c(C)cc1/N=C/N(C)C1CCCCC1] | Example 1; Example 2D; Example 3A; Example 4A | White solid |
| 41 | 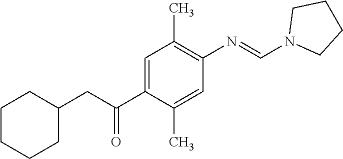 [SMILES: Cc1cc(C(=O)CC2CCCCC2)c(C)cc1/N=C/N1CCCC1] | Example 1; Example 2D; Example 3A; Example 4A | Yellow oil |
| 42 | 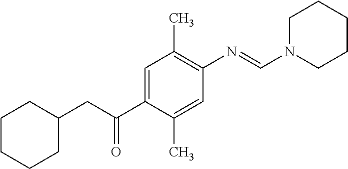 [SMILES: Cc1cc(C(=O)CC2CCCCC2)c(C)cc1/N=C/N1CCCCC1] | Example 1; Example 2D; Example 3A; Example 4A | Yellow oil |
| 43 | 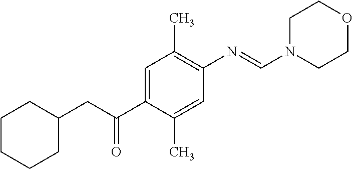 [SMILES: Cc1cc(C(=O)CC2CCCCC2)c(C)cc1/N=C/N1CCOCC1] | Example 1; Example 2D; Example 3A; Example 4A | Yellow solid |
| 44 | 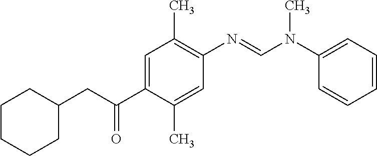 [SMILES: Cc1cc(C(=O)CC2CCCCC2)c(C)cc1/N=C/N(C)c1ccccc1] | Example 1; Example 2D; Example 3A; Example 4A | Yellow oil |
| 45 | 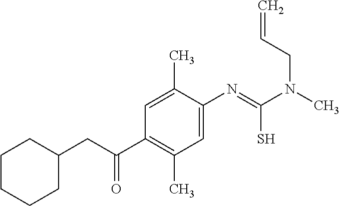 [SMILES: C=CCN(C)/C(S)=N/c1cc(C)c(C(=O)CC2CCCCC2)cc1C] | Example 1; Example 2D; Example 3A; Example 4E | Yellow oil |
| 46 | 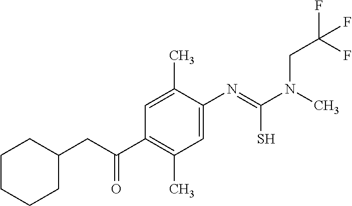 [SMILES: Cc1cc(C(=O)CC2CCCCC2)c(C)cc1/N=C(\S)N(C)CC(F)(F)F] | Example 1; Example 2D; Example 3A; Example 4E | White solid |
| 47 | 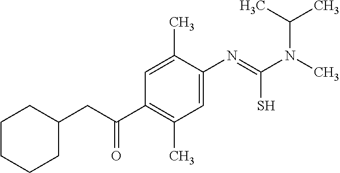 [SMILES: Cc1cc(C(=O)CC2CCCCC2)c(C)cc1/N=C(\S)N(C)C(C)C] | Example 1; Example 2D; Example 3A; Example 4E | Yellow oil |
| 48 | 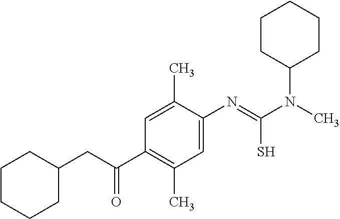 [SMILES: Cc1cc(C(=O)CC2CCCCC2)c(C)cc1/N=C(\S)N(C)C1CCCCC1] | Example 1; Example 2D; Example 3A; Example 4E | Yellow oil |
| 49 | 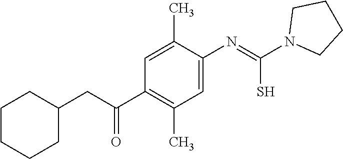 [SMILES: Cc1cc(C(=O)CC2CCCCC2)c(C)cc1/N=C(\S)N1CCCC1] | Example 1; Example 2D; Example 3A; Example 4E | White solid |
| 50 | 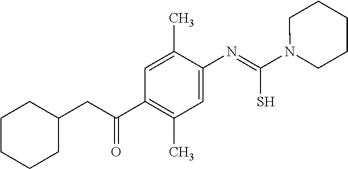 [SMILES: Cc1cc(C(=O)CC2CCCCC2)c(C)cc1/N=C(\S)N1CCCCC1] | Example 1; Example 2D; Example 3A; Example 4E | Yellow oil |
| 51 | 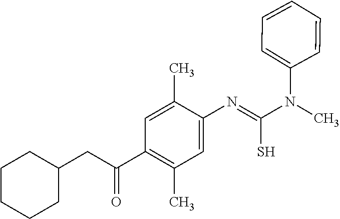 [SMILES: Cc1cc(C(=O)CC2CCCCC2)c(C)cc1/N=C(\S)N(C)c1ccccc1] | Example 1; Example 2D; Example 3A; Example 4E | White solid |
| 52 | 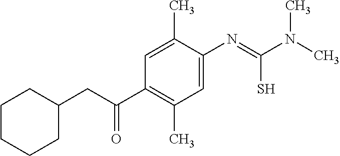 [SMILES: Cc1cc(C(=O)CC2CCCCC2)c(C)cc1/N=C(\S)N(C)C] | Example 1; Example 2D; Example 3A; Example 4E | White solid |
| 53 | 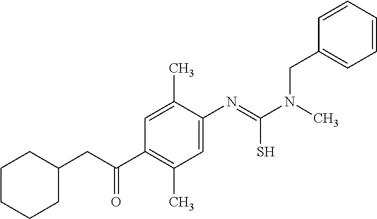 [SMILES: Cc1cc(C(=O)CC2CCCCC2)c(C)cc1/N=C(\S)N(C)Cc1ccccc1] | Example 1; Example 2D; Example 3A; Example 4E | White solid |
| 54 | 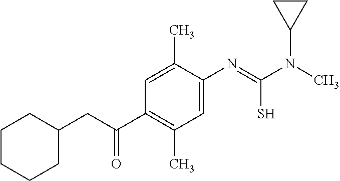 [SMILES: Cc1cc(C(=O)CC2CCCCC2)c(C)cc1/N=C(\S)N(C)C1CC1] | Example 1; Example 2D; Example 3A; Example 4E | White solid |
| 55 | 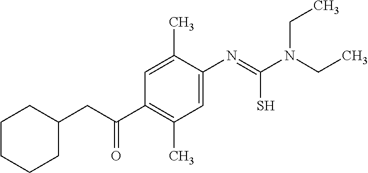 [SMILES: CCN(CC)/C(S)=N/c1cc(C)c(C(=O)CC2CCCCC2)cc1C] | Example 1; Example 2D; Example 3A; Example 4E | Yellow oil |
| 56 | 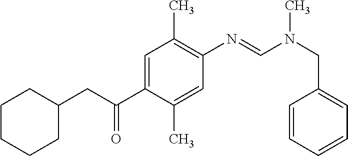 [SMILES: Cc1cc(C(=O)CC2CCCCC2)c(C)cc1/N=C/N(C)Cc1ccccc1] | Example 1; Example 2D; Example 3A; Example 4A | Yellow oil |
| 57 | 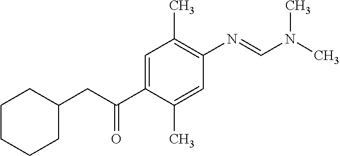 [SMILES: Cc1cc(C(=O)CC2CCCCC2)c(C)cc1/N=C/N(C)C] | Example 1; Example 2D; Example 3A; Example 4A | Light yellow oil |
| 58 | 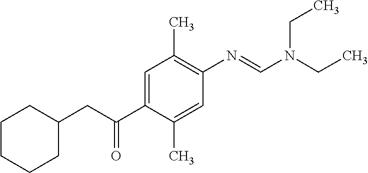 [SMILES: CCN(/C=N/c1cc(C)c(C(=O)CC2CCCCC2)cc1C)CC] | Example 1; Example 2D; Example 3A; Example 4A | White powder |
| 59 | 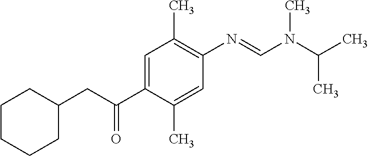 [SMILES: Cc1cc(C(=O)CC2CCCCC2)c(C)cc1/N=C/N(C)C(C)C] | Example 1; Example 2D; Example 3A; Example 4A | Yellow oil |
| 60 | 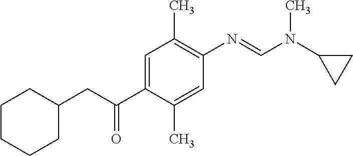 [SMILES: Cc1cc(C(=O)CC2CCCCC2)c(C)cc1/N=C/N(C)C1CC1] | Example 1; Example 2D; Example 3A; Example 4A | Yellow oil |
| 61 | 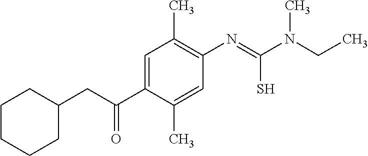 [SMILES: CCN(C)/C(S)=N/c1cc(C)c(C(=O)CC2CCCCC2)cc1C] | Example 1; Example 2D; Example 3A; Example 4E | White solid |
| 62 | 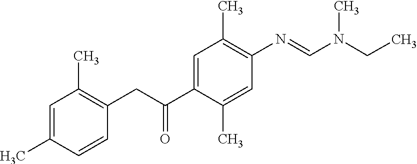 [SMILES: CCN(C)/C=N/c1cc(C)c(C(=O)Cc2ccc(C)cc2C)cc1C] | Example 1; Example 2B; Example 3A; Example 4D | Yellow solid |
| 63 | 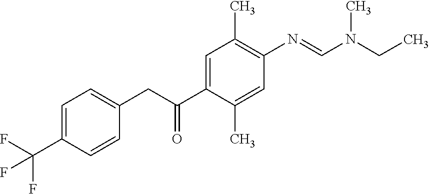 [SMILES: CCN(C)/C=N/c1cc(C)c(C(=O)Cc2ccc(C(F)(F)F)cc2)cc1C] | Example 1; Example 2B; Example 3A; Example 4D | Yellow solid |
| 64 | 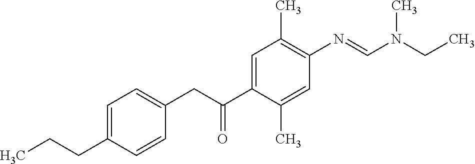 [SMILES: CCCc1ccc(CC(=O)c2cc(C)c(/N=C/N(C)CC)cc2C)cc1] | Example 1; Example 2B; Example 3A; Example 4D | Amber film |
| 65 | 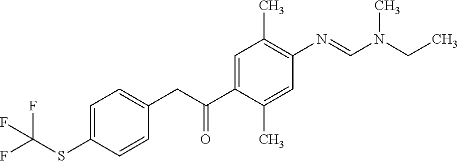 [SMILES: CCN(C)/C=N/c1cc(C)c(C(=O)Cc2ccc(SC(F)(F)F)cc2)cc1C] | Example 1; Example 2B; Example 3A; Example 4D | Yellow solid |
| 66 | 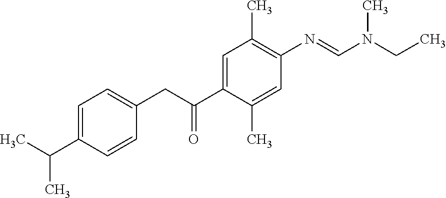 [SMILES: CCN(C)/C=N/c1cc(C)c(C(=O)Cc2ccc(C(C)C)cc2)cc1C] | Example 1; Example 2B; Example 3A; Example 4D | Yellow film |
| 67 | 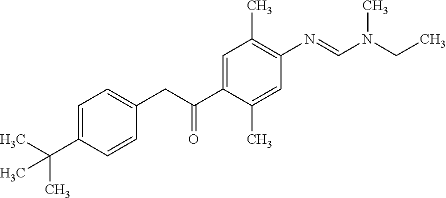 [SMILES: CCN(C)/C=N/c1cc(C)c(C(=O)Cc2ccc(C(C)(C)C)cc2)cc1C] | Example 1; Example 2B; Example 3A; Example 4D | Yellow film |
| 68 | 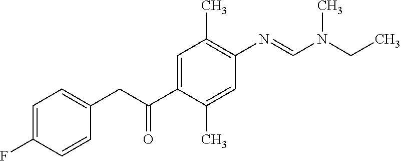 [SMILES: CCN(C)/C=N/c1cc(C)c(C(=O)Cc2ccc(F)cc2)cc1C] | Example 1; Example 2B; Example 3A; Example 4D | Yellow film |
| 69 | 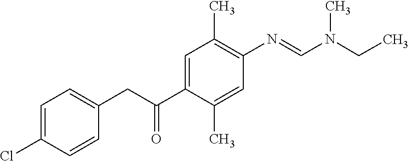 [SMILES: CCN(C)/C=N/c1cc(C)c(C(=O)Cc2ccc(Cl)cc2)cc1C] | Example 1; Example 2A; Example 3A; Example 4D | Yellow solid |
| 70 | 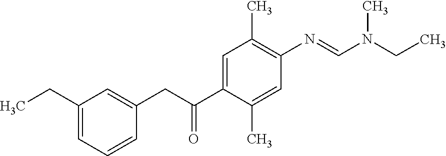 [SMILES: CCc1cccc(CC(=O)c2cc(C)c(/N=C/N(C)CC)cc2C)c1] | Example 1; Example 2B; Example 3A; Example 4D | Yellow film |
| 71 | 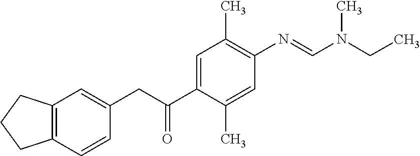 [SMILES: CCN(C)/C=N/c1cc(C)c(C(=O)Cc2ccc3c(c2)CCC3)cc1C] | Example 1; Example 2B; Example 3A; Example 4D | Yellow film |
| 72 | 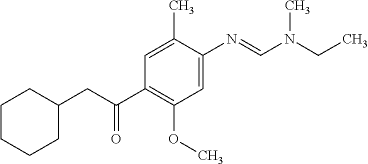 [SMILES: CCN(C)/C=N/c1cc(OC)c(C(=O)CC2CCCCC2)cc1C] | Example 1; Example 2D; Example 3A; Example 4D | Yellow semi-solid |
| 73 | 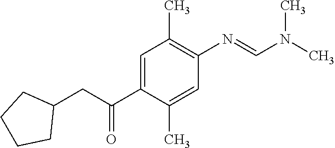 [SMILES: Cc1cc(C(=O)CC2CCCC2)c(C)cc1/N=C/N(C)C] | Example 1; Example 2D; Example 3A; Example 4B | Colorless film |
| 74 | 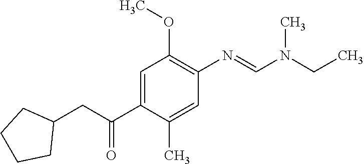 [SMILES: CCN(C)/C=N/c1cc(C)c(C(=O)CC2CCCC2)cc1OC] | Example 1; Example 2A; Example 3A; Example 4D | Colorless film |
| 75 | 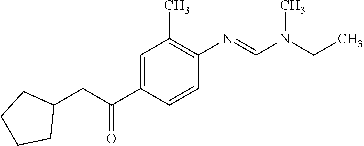 [SMILES: CCN(C)/C=N/c1ccc(C(=O)CC2CCCC2)cc1C] | Example 1; Example 2A; Example 3A; Example 4D | Colorless film |
| 76 | 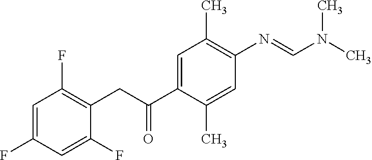 [SMILES: Cc1cc(C(=O)Cc2c(F)cc(F)cc2F)c(C)cc1/N=C/N(C)C] | Example 1; Example 2E; Example 3A; Example 4A | Colorless film |
| 77 | 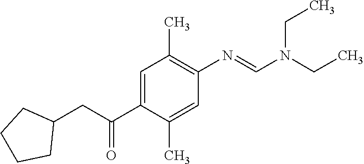 [SMILES: CCN(/C=N/c1cc(C)c(C(=O)CC2CCCC2)cc1C)CC] | Example 1; Example 2D; Example 3A; Example 4A | White solid |
| 78 | 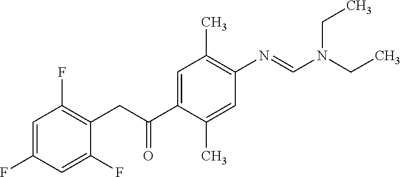 [SMILES: CCN(/C=N/c1cc(C)c(C(=O)Cc2c(F)cc(F)cc2F)cc1C)CC] | Example 1; Example 2E; Example 3A; Example 4A | Yellow solid |
| 79 | 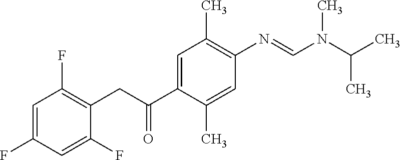 [SMILES: Cc1cc(C(=O)Cc2c(F)cc(F)cc2F)c(C)cc1/N=C/N(C)C(C)C] | Example 1; Example 2E; Example 3A; Example 4A | Yellow solid |
| 80 | 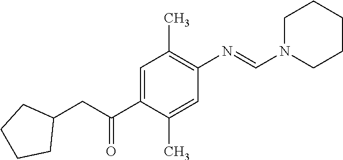 [SMILES: Cc1cc(C(=O)CC2CCCC2)c(C)cc1/N=C/N1CCCCC1] | Example 1; Example 2D; Example 3A; Example 4A | Colorless film |
| 81 | 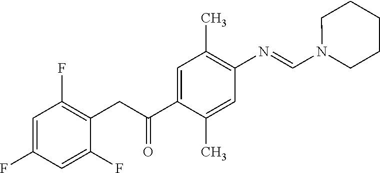 [SMILES: Cc1cc(C(=O)Cc2c(F)cc(F)cc2F)c(C)cc1/N=C/N1CCCCC1] | Example 1; Example 2E; Example 3A; Example 4A | Colorless film |
| 82 | 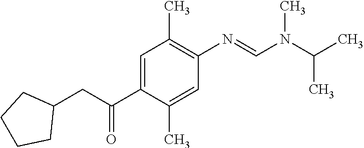 [SMILES: Cc1cc(C(=O)CC2CCCC2)c(C)cc1/N=C/N(C)C(C)C] | Example 1; Example 2D; Example 3A; Example 4A | White solid |
| 83 | 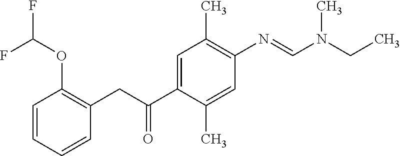 [SMILES: CCN(C)/C=N/c1cc(C)c(C(=O)Cc2ccccc2OC(F)F)cc1C] | Example 1; Example 2B; Example 3A; Example 4D | Yellow film |
| 84 | 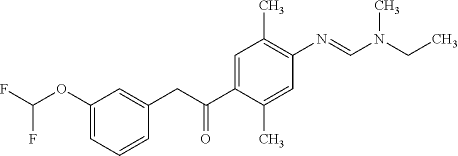 [SMILES: CCN(C)/C=N/c1cc(C)c(C(=O)Cc2cccc(OC(F)F)c2)cc1C] | Example 1; Example 2B; Example 3A; Example 4D | Yellow film |
| 85 | 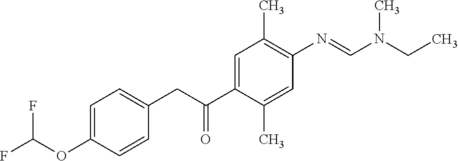 [SMILES: CCN(C)/C=N/c1cc(C)c(C(=O)Cc2ccc(OC(F)F)cc2)cc1C] | Example 1; Example 2B; Example 3A; Example 4D | Yellow film |
| 86 | 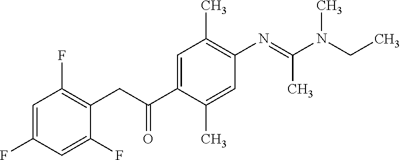 [SMILES: CCN(C)/C(C)=N/c1cc(C)c(C(=O)Cc2c(F)cc(F)cc2F)cc1C] | Example 1; Example 2E; Example 3A; Example 4C | Yellow film |
| 87 | 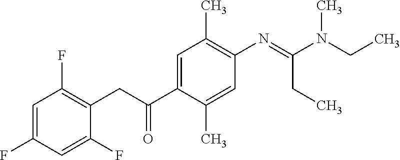 [SMILES: CC/C(=N\c1cc(C)c(C(=O)Cc2c(F)cc(F)cc2F)cc1C)N(C)CC] | Example 1; Example 2E; Example 3A; Example 4C | Amber film |
| 88 | 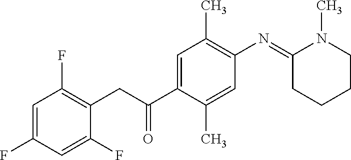 [SMILES: Cc1cc(C(=O)Cc2c(F)cc(F)cc2F)c(C)cc1/N=C1\CCCCN1C] | Example 1; Example 2E; Example 3A; Example 4C | Tan solid |
| 89 | 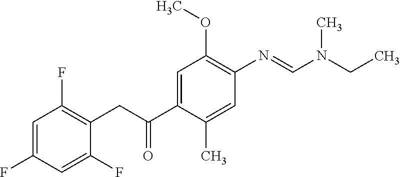 [SMILES: CCN(C)/C=N/c1cc(C)c(C(=O)Cc2c(F)cc(F)cc2F)cc1OC] | Example 1; Example 2B; Example 3A; Example 4D | Tan film |
| 90 | 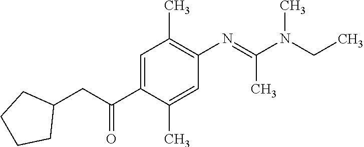 [SMILES: CCN(C)/C(C)=N/c1cc(C)c(C(=O)CC2CCCC2)cc1C] | Example 1; Example 2D; Example 3A; Example 4C | Gold film |
| 91 | 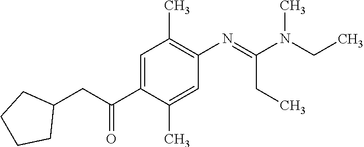 [SMILES: CC/C(=N\c1cc(C)c(C(=O)CC2CCCC2)cc1C)N(C)CC] | Example 1; Example 2D; Example 3A; Example 4C | Gold film |
| 92 | 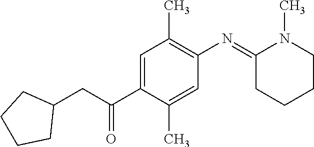 [SMILES: Cc1cc(C(=O)CC2CCCC2)c(C)cc1/N=C1\CCCCN1C] | Example 1; Example 2D; Example 3A; Example 4C | Colorless film |
| 93 | 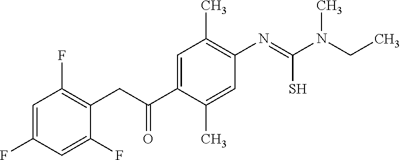 [SMILES: CCN(C)/C(S)=N/c1cc(C)c(C(=O)Cc2c(F)cc(F)cc2F)cc1C] | Example 1; Example 2E; Example 3A; Example 4E | White solid |
| 94 | 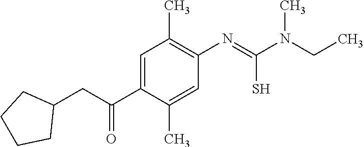 [SMILES: CCN(C)/C(S)=N/c1cc(C)c(C(=O)CC2CCCC2)cc1C] | Example 1; Example 2D; Example 3A; Example 4E | Colorless film |
| 95 | 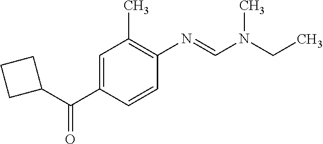 [SMILES: CCN(C)/C=N/c1ccc(C(=O)C2CCC2)cc1C] | Example 7; Example 6; Example 8 | Yellow oil |
| 96 | 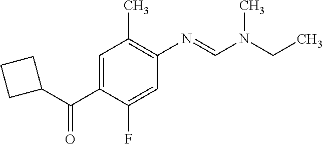 [SMILES: CCN(C)/C=N/c1cc(F)c(C(=O)C2CCC2)cc1C] | Example 5; Example 6; Example 7; Example 8 | Yellow oil |
| 97 | 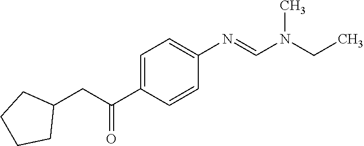 [SMILES: CCN(C)/C=N/c1ccc(C(=O)CC2CCCC2)cc1] | Example 1; Example 2A; Example 3A; Example 4D | Tan solid |
| 98 | 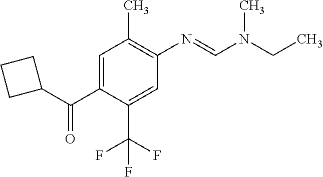 [SMILES: CCN(C)/C=N/c1cc(C(F)(F)F)c(C(=O)C2CCC2)cc1C] | Example 5; Example 6; Example 7; Example 8 | Yellow oil |
| 99 | 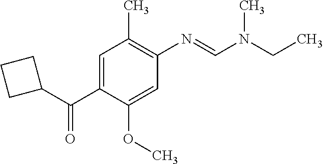 [SMILES: CCN(C)/C=N/c1cc(OC)c(C(=O)C2CCC2)cc1C] | Example 6; Example 7; Example 8 | Yellow Oil |
| 100 | 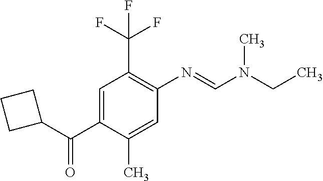 [SMILES: CCN(C)/C=N/c1cc(C)c(C(=O)C2CCC2)cc1C(F)(F)F] | Example 5; Example 6; Example 7; Example 8 | Colorless oil |
| 101 | 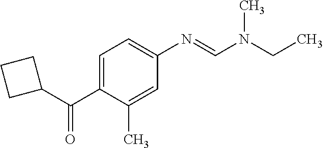 [SMILES: CCN(C)/C=N/c1ccc(C(=O)C2CCC2)c(C)c1] | Example 6; Example 7; Example 8 | Yellow oil |
| 102 | 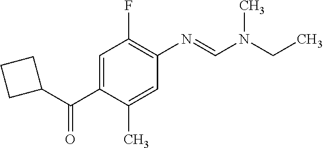 [SMILES: CCN(C)/C=N/c1cc(C)c(C(=O)C2CCC2)cc1F] | Example 6; Example 7; Example 8 | Yellow oil |
| 103 | 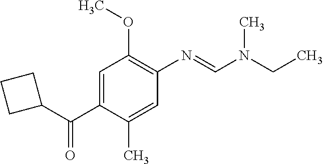 [SMILES: CCN(C)/C=N/c1cc(C)c(C(=O)C2CCC2)cc1OC] | Example 6; Example 7; Example 8 | Yellow oil |
| 104 | 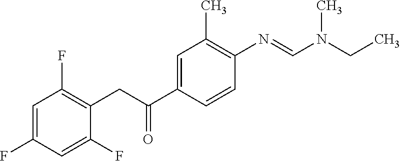 [SMILES: CCN(C)/C=N/c1ccc(C(=O)Cc2c(F)cc(F)cc2F)cc1C] | Example 1; Example 2B; Example 3A; Example 4D | Colorless film |
| 105 | 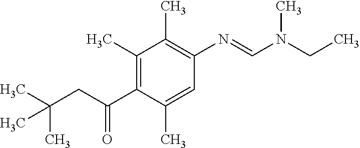 [SMILES: CCN(C)/C=N/c1cc(C)c(C(=O)CC(C)(C)C)c(C)c1C] | Example 5; Example 6; Example 9; Example 10 | Colorless oil |

TABLE 2
Analytical Characterization Data

| Cmpd. | | Melting | | NMR |
|---|---|---|---|---|
| No. | IR (cm⁻¹) | Point (° C.) | MASS | (¹H, ¹³C or ¹⁹F) |
| 1 | | 101-105 | ESIMS m/z 323 | ¹H NMR (400 MHz, DMSO-d₆) δ |
| | | | ([M + H]⁺) | 7.84-7.60 (m, 2H), 7.20-7.05 (m, |
| | | | | 4H), 6.69 (br s, 1H), 4.27 (s, 2H), |
| | | | | 3.55-3.36 (m, 2H), 3.00-2.85 (m, |
| | | | | 3H), 2.35 (s, 3H), 2.21 (s, 3H), 2.15 |
| | | | | (s, 3H), 1.14 (t, J = 7.0 Hz, 3H); |
| | | | | ¹³C NMR (101 MHz, DMSO-d₆) δ |
| | | | | 199.11, 153.72, 152.67, 137.08, |
| | | | | 136.74, 135.03, 131.65, 130.52, |
| | | | | 129.96, 129.72, 127.73, 126.46, |
| | | | | 125.59, 121.47, 46.88, 45.16, 31.48, |
| | | | | 21.29, 19.28, 17.43, 14.07 |
| 2 | | 43-46 | ESIMS m/z 309 | ¹H NMR (400 MHz, DMSO-d₆) δ |
| | | | ([M + H]⁺) | 7.85-7.62 (m, 2H), 7.35-7.15 (m, |
| | | | | 5H), 6.68 (s, 1H), 4.23 (s, 2H), 3.57- |
| | | | | 3.35 (m, 2H), 3.06-2.82 (m, 3H), |
| | | | | 2.35 (s, 3H), 2.20 (s, 3H), 1.13 (t, J = |
| | | | | 6.8 Hz, 3H); |
| | | | | ¹³C NMR (101 MHz, DMSO-d₆) δ |
| | | | | 199.05, 153.76, 152.68, 137.48, |
| | | | | 135.98, 132.06, 129.53, 129.36, |
| | | | | 128.18, 127.70, 126.19, 121.50, |
| | | | | 46.88, 46.75, 31.46, 21.45, 17.42, |
| | | | | 14.04 |
| 3 | | 69-71 | ESIMS m/z 315 | ¹H NMR (400 MHz, DMSO-d₆) δ |
| | | | ([M + H]⁺) | 7.80-7.60 (m, 1H), 7.56 (s, 1H), |
| | | | | 6.66 (s, 1H), 3.52-3.35 (m, 2H), |
| | | | | 3.08-2.88 (m, 3H), 2.72 (d, J = 6.8 |
| | | | | Hz, 2H), 2.36 (s, 3H), 2.18 (s, 3H), |
| | | | | 1.85-1.72 (m 1H), 1.72-1.52 (m, |
| | | | | 5H), 1.31-1.05 (m, 6H), 1.05- |
| | | | | 0.82 (m, 2H); |
| | | | | ¹³C NMR (101 MHz, DMSO-d₆) δ |
| | | | | 201.75, 153.45, 152.59, 136.63, |
| | | | | 131.35, 130.71, 127.65, 121.44, |
| | | | | 47.70, 46.85, 34.31, 32.65, 31.45, |
| | | | | 25.84, 25.70, 21.24, 17.38, 14.05 |
| 4 | 1630 (C═O) | | ESIMS m/z 301 | ¹H NMR (400 MHz, DMSO-d₆) δ |
| | | | ([M + H]⁺) | 7.80-7.60 (m, 1H), 7.57 (s, 1H), |
| | | | | 6.66 (s, 1H), 3.52-3.35 (m, 2H), |
| | | | | 3.02-2.81 (m, 3H), 2.88 (d, J = 7.2 |
| | | | | Hz, 2H), 2.36 (s, 3H), 2.25-2.15 |
| | | | | (m, 4H), 1.81-1.68 (m, 2H), 1.65- |
| | | | | 1.53 (m, 2H), 1.53-1.42 (m, 2H), |
| | | | | 1.18-1.05 (m, 5H); |
| | | | | ¹³C NMR (101 MHz, DMSO-d₆) δ |
| | | | | 201.97, 153.42, 152.59, 136.62, |
| | | | | 131.33, 130.52, 127.64, 121.43, |
| | | | | 46.85, 46.39, 36.07, 32.04, 31.45, |
| | | | | 24.48, 21.23, 17.37, 14.06 |
| 5 | | 73-76 | ESIMS m/z 377 | ¹H NMR (400 MHz, DMSO-d₆) δ |
| | | | ([M + H]⁺) | 7.84 (s, 1H), 7.82-7.66 (m, 1H), |
| | | | | 7.66-7.49 (m, 4H), 6.78-6.63 (m, |
| | | | | 1H), 4.43 (s, 2H), 3.56-3.35 (m, |
| | | | | 2H), 3.12-2.88 (m, 3H), 2.37 (s, |
| | | | | 3H), 2.22 (s, 3H), 1.20-1.09 (m, |
| | | | | 3H); |
| | | | | ¹³CNMR (101 MHz, DMSO-d₆) δ |
| | | | | 198.29, 154.00, 152.80, 137.68, |
| | | | | 137.57, 134.15, 132.16, 129.22, |
| | | | | 128.95, 128.63, 128.32, 127.84, |
| | | | | 126.49, 122.98, 121.57, 46.95, 45.99, |
| | | | | 31.52, 21.62, 17.64, 14.10; |
| | | | | ¹⁹F NMR (376 MHz, DMSO-d₆) |
| | | | | δ −60.98 |
| 6 | 1631 (C═O) | | ESIMS m/z 337 | ¹H NMR (400 MHz, DMSO-d₆) δ |
| | | | ([M + H]⁺) | 7.89-7.70 (m, 2H), 7.20-7.08 (m, |
| | | | | 4H), 6.67 (br s, 1H), 4.18 (s, 2H), |
| | | | | 3.50-3.36 (m, 2H), 3.10-2.90 (m, |
| | | | | 3H), 2.55 (q, J = 7.6 Hz, 2H), 2.34 (s, |
| | | | | 3H), 2.19 (s, 3H), 1.20-1.10 (m, |
| | | | | 6H); |
| | | | | ¹³C NMR (101 MHz, DMSO-d₆) δ |
| | | | | 199.24, 153.71, 152.66, 141.54, |
| | | | | 137.46, 133.11, 132.06, 129.64, |
| | | | | 128.77, 127.67, 127.60, 121.50, |
| | | | | 46.87, 46.39, 31.46, 27.74, 21.44, |
| | | | | 17.42, 15.58, 14.04 |
| 7 | | 102-105 | ESIMS m/z 341 | ¹H NMR (300 MHz, DMSO-d₆) δ |
| | | | ([M + H]⁺) | 7.86-7.62 (m, 2H), 7.22-7.10 (m, |
| | | | | 1H), 7.05-6.88 (m, 2H), 6.69 (s, |
| | | | | 1H), 4.24 (s, 2H), 3.58-3.32 (m, |
| | | | | 2H), 3.08-2.82 (m, 3H), 2.35 (s, |
| | | | | 3H), 2.30 (s, 3H), 2.20 (s, 3H), 1.14 |
| | | | | (t, J = 7.2 Hz, 3H); |
| | | | | ¹³C NMR (75 MHz, DMSO-d₆) δ |
| | | | | 197.68, 162.21, 153.84, 152.68, |
| | | | | 138.50, 137.33, 131.87, 131.76, |
| | | | | 129.32, 127.77, 124.69, 121.46, |
| | | | | 120.19, 115.45, 46.89, 40.34, 31.48, |
| | | | | 21.63, 20.50, 17.40, 14.06; |
| | | | | ¹⁹F NMR (282 MHz, DMSO-d₆) |
| | | | | δ −117.94 |
| 8 | | 70-74 | ESIMS m/z 393 | ¹H NMR (400 MHz, DMSO-d₆) δ |
| | | | ([M + H]⁺) | 7.88-7.62 (m, 2H), 7.45-7.21 (m, |
| | | | | 4H), 6.75-6.58 (m, 1H), 4.43 (s, |
| | | | | 2H), 3.56-3.35 (m, 2H), 3.12-2.88 |
| | | | | (m, 3H), 2.36 (s, 3H), 2.21 (s, 3H), |
| | | | | 1.14 (t, J = 6.4 Hz, 3H); |
| | | | | ¹⁹F NMR (376 MHz, DMSO-d₆) |
| | | | | δ −56.83 |
| 9 | | 90-93 | ESIMS m/z 323 | ¹H NMR (400 MHz, DMSO-d₆) δ |
| | | | ([M + H]⁺) | 7.82-7.68 (m, 2H), 7.14-7.06 (m, |
| | | | | 4H), 6.66 (s, 1H), 4.17 (s, 2H), 3.52- |
| | | | | 3.36 (m, 2H), 3.06-2.88 (m, 3H), |
| | | | | 2.33 (s, 3H), 2.22 (s, 3H), 2.16 (s, |
| | | | | 3H), 1.19-1.08 (m, 3H) |
| 10 | 1630 (C═O) | 53-57 | ESIMS m/z 377 | ¹H NMR (400 MHz, DMSO-d₆) δ |
| | | | ([M + H]⁺) | 7.78-7.50 (m, 6H), 6.75 (d, J = 8.0 |
| | | | | Hz, 1H), 4.39 (s, 2H), 3.50-3.35 (m, |
| | | | | 2H), 3.08-2.90 (m, 3H), 2.25 (s, |
| | | | | 3H), 2.18 (s, 3H), 1.14 (t, J = 7.2 Hz, |
| | | | | 3H); |
| | | | | ¹⁹F NMR (376 MHz, DMSO-d₆) |
| | | | | δ = −60.95 |
| 11 | 1628 (C═O) | | ESIMS m/z 341 | ¹H NMR (400 MHz, DMSO-d₆) δ |
| | | | ([M + H]⁺) | 7.78-7.63 (m, 1H), 7.58 (d, J = 8.0 |
| | | | | Hz, 1H), 7.18 (t, J = 7.6 Hz, 1H), 7.02- |
| | | | | 6.92 (m, 2H), 6.72 (d, J = 8.0 Hz, |
| | | | | 1H), 4.20 (s, 2H), 3.55-3.35 (m, |
| | | | | 2H), 3.10-2.88 (m, 3H), 2.29 (s, |
| | | | | 3H), 2.24 (s, 3H), 2.18 (s, 3H), 1.14 |
| | | | | (t, J = 6.8 Hz, 3H); |
| | | | | ¹⁹F NMR (376 MHz, DMSO-d₆) |
| | | | | δ −117.88 |
| 12 | 1628 (C═O) | | ESIMS m/z 323 | ¹H NMR (400 MHz, DMSO-d₆) δ |
| | | | ([M + H]⁺) | 7.77-7.62 (m, 1H), 7.58 (d, J = 8.4 |
| | | | | Hz, 1H), 7.09 (s, 4H), 6.70 (d, J = 8.4 |
| | | | | Hz, 1H), 4.14 (s, 2H), 3.52-3.34 (m, |
| | | | | 2H), 3.04-2.86 (m, 3H), 2.25 (s, |
| | | | | 3H), 2.21 (s, 3H), 2.16 (s, 3H), 1.13 |
| | | | | (t, J = 7.2 Hz, 3H) |
| 13 | 1630 (C═O) | 52-56 | ESIMS m/z 315 | ¹H NMR (400 MHz, DMSO-d₆) δ |
| | | | ([M + H]⁺) | 7.79-7.58 (m, 1H), 7.53 (d, J = 8.4 |
| | | | | Hz, 1H), 6.73 (d, J = 8.0 Hz, 1H), |
| | | | | 3.53-3.35 (m, 2H), 3.16 (t, J = 7.2 |
| | | | | Hz, 2H), 3.05-2.90 (m, 3H), 2.68- |
| | | | | 2.53 (m, 2H), 2.28 (s, 3H), 2.19 (s, |
| | | | | 3H), 1.14 (t, J = 6.8 Hz, 3H); |
| | | | | ¹⁹F NMR (376 MHz, DMSO-d₆) |
| | | | | δ −64.57 |
| 14 | | 49-52 | ESIMS m/z 329 | ¹H NMR (400 MHz, DMSO-d₆) δ |
| | | | ([M + H]⁺) | 7.77-7.53 (m, 1H), 7.45 (d, J = 8.4 |
| | | | | Hz, 1H), 6.72 (d, J = 8.0 Hz, 1H), |
| | | | | 3.57-3.35 (m, 2H), 3.10-2.90 (m, |
| | | | | 5H), 2.40-2.24 (m, 2H), 2.23 (s, |
| | | | | 3H), 2.19 (s, 3H), 1.87-1.70 (m, |
| | | | | 2H), 1.14 (t, J = 7.2 Hz, 3H); |
| | | | | ¹⁹F NMR (376 MHz, DMSO-d₆) |
| | | | | δ −64.94 |
| 15 | 1627 (C═O) | 68-72 | ESIMS m/z 315 | ¹H NMR (400 MHz, DMSO-d₆) δ |
| | | | ([M + H]⁺) | 7.75-7.52 (m, 1H), 7.39 (d, J = 8.4 |
| | | | | Hz, 1H), 6.69 (d, J = 8.0 Hz, 1H), |
| | | | | 3.50-3.35 (m, 2H), 3.05-2.90 (m, |
| | | | | 3H), 2.70 (d, J = 6.8 Hz, 2H), 2.24 (s, |
| | | | | 3H), 2.18 (s, 3H), 1.85-1.70 (m, |
| | | | | 1H), 1.69-1.52 (m, 5H), 1.30-1.03 |
| | | | | (m, 6H), 1.02-0.88 (m, 2H) |
| 16 | 1630 (C═O) | | ESIMS m/z 309 | ¹H NMR (400 MHz, DMSO-d₆) δ |
| | | | ([M + H]⁺) | 7.78-7.53 (m, 2H), 7.35-7.26 (m, |
| | | | | 2H), 7.25-7.16 (m, 3H), 6.71 (d, J = |
| | | | | 8.0 Hz, 1H), 4.20 (s, 2H), 3.55-3.35 |
| | | | | (m, 2H), 3.08-2.90 (m, 3H), 2.22 (s, |
| | | | | 3H), 2.16 (s, 3H), 1.13 (t, J = 7.2 Hz, |
| | | | | 3H) |
| 17 | | | ESIMS m/z 287 | ¹H NMR (500 MHz, CDCl₃) δ 7.51 |
| | | | ([M + H]⁺) | (s, 1H), 7.50-7.44 (m, 1H), 6.57 (s, |
| | | | | 1H), 3.63 (p, J = 7.9 Hz, 1H), 3.56- |
| | | | | 3.23 (m, 2H), 3.01 (s, 3H), 2.46 (s, |
| | | | | 3H), 2.27 (s, 3H), 1.92-1.80 (m, |
| | | | | 4H), 1.77-1.67 (m, 2H), 1.67-1.57 |
| | | | | (m, 2H), 1.21 (t, J = 7.2 Hz, 3H); |
| | | | | ¹³C NMR (126 MHz, CDCl₃) δ |
| | | | | 205.83, 153.61, 151.76, 137.98, |
| | | | | 131.60, 131.44, 128.35, 122.23, |
| | | | | 48.45, 47.80, 31.98, 30.26, 26.31, |
| | | | | 21.60, 17.69, 14.32 |
| 18 | | | ESIMS m/z 327 | ¹H NMR (500 MHz, CDCl₃) δ 7.53- |
| | | | ([M + H]⁺) | 7.44 (m, 1H), 7.27-7.23 (m, 1H), |
| | | | | 7.17 (tdd, J = 7.4, 5.2, 1.8 Hz, 1H), |
| | | | | 7.07-6.99 (m, 1H), 6.57 (s, 1H), |
| | | | | 3.58-3.29 (m, 2H), 3.22 (dd, J = |
| | | | | 8.6, 6.8 Hz, 2H), 3.07-3.03 (m, |
| | | | | 2H), 3.01 (s, 3H), 2.50 (s, 3H), 2.24 |
| | | | | (s, 3H), 1.22 (t, J = 7.1 Hz, 3H); |
| | | | | ¹⁹F NMR (471 MHz, CDCl₃) |
| | | | | δ −118.43 (p, J = 8.4, 8.0 Hz) |
| 19 | | | ESIMS m/z 315 | ¹H NMR (500 MHz, CDCl₃) δ 7.56 |
| | | | ([M + H]⁺) | (s, 1H), 7.51 (s, 1H), 6.59 (s, 1H), |
| | | | | 3.60-3.27 (m, 2H), 3.22-3.14 (m, |
| | | | | 2H), 3.02 (s, 3H), 2.55 (tdd, J = |
| | | | | 10.9, 7.8, 5.3 Hz, 2H), 2.50 (s, 3H), |
| | | | | 2.28 (s, 3H), 1.23 (t, J = 7.2 Hz, |
| | | | | 3H); |
| | | | | ¹⁹F NMR (471 MHz, CDCl₃) |
| | | | | δ −66.27 |
| 20 | | | ESIMS m/z 345 | ¹H NMR (500 MHz, CDCl₃) δ 7.69 |
| | | | ([M + H]⁺) | (s, 1H), 7.47 (d, J = 39.1 Hz, 1H), |
| | | | | 7.23-7.13 (m, 1H), 6.87-6.78 (m, |
| | | | | 2H), 6.59 (s, 1H), 4.23 (s, 2H), 3.60- |
| | | | | 3.26 (m, 2H), 3.02 (s, 3H), 2.49 (s, |
| | | | | 3H), 2.29 (s, 3H), 1.22 (t, J = 7.1 Hz, |
| | | | | 3H); |
| | | | | ¹⁹F NMR (471 MHz, CDCl₃) |
| | | | | δ −112.40 (p, J = 7.6 Hz), −112.89 |
| | | | | (q, J = 8.3 Hz) |
| 21 | | | ESIMS m/z 289 | ¹H NMR (500 MHz, CDCl₃) δ 7.44 |
| | | | ([M + H]⁺) | (m, 2H), 6.56 (s, 1H), 3.40 (m, 2H), |
| | | | | 2.99 (s, 3H), 2.77 (s, 2H), 2.45 (s, |
| | | | | 3H), 2.26 (s, 3H), 1.20 (t, J = 7.2 Hz, |
| | | | | 3H), 1.02 (s, 9H); |
| | | | | ¹³C NMR (126 MHz, CDCl₃) δ |
| | | | | 203.73, 153.54, 151.75, 137.09, |
| | | | | 133.33, 131.60, 128.21, 122.28, |
| | | | | 52.86, 47.76, 31.92, 31.71, 30.21, |
| | | | | 21.43, 17.66, 14.25 |
| 22 | | | ESIMS m/z 289 | ¹H NMR (500 MHz, CDCl₃) δ 7.53 |
| | | | ([M + H]⁺) | (s, 1H), 7.46 (m, 1H), 6.57 (s, 1H), |
| | | | | 3.58-3.25 (m, 2H), 3.00 (s, 3H), |
| | | | | 2.90-2.84 (m, 2H), 2.48 (s, 3H), |
| | | | | 2.27 (s, 3H), 1.60 (dq, J = 14.1, 6.7 |
| | | | | Hz, 3H), 1.21 (t, J = 7.2 Hz, 3H), 0.93 |
| | | | | (d, J = 6.2 Hz, 6H); |
| | | | | ¹³C NMR (126 MHz, CDCl₃) δ |
| | | | | 203.22, 153.89, 151.77, 137.96, |
| | | | | 131.51, 131.26, 128.44, 122.32, |
| | | | | 47.81, 38.83, 33.75, 31.94, 27.88, |
| | | | | 22.52, 21.73, 17.67, 14.30 |
| 23 | | | ESIMS m/z 363 | ¹H NMR (500 MHz, CDCl₃) δ 7.71 |
| | | | ([M + H]⁺) | (s, 1H), 7.57-7.37 (m, 1H), 6.70- |
| | | | | 6.63 (m, 2H), 6.60 (s, 1H), 4.26 (s, |
| | | | | 2H), 3.62-3.24 (m, 2H), 3.02 (s, |
| | | | | 3H), 2.49 (s, 3H), 2.30 (s, 3H), 1.21 |
| | | | | (t, J = 7.2 Hz, 3H); |
| | | | | ¹⁹F NMR (471 MHz, CDCl₃) |
| | | | | δ −110.19 (d, J= 5.7 Hz), −111.66 |
| | | | | (d, J = 6.4 Hz) |
| 24 | | | ESIMS m/z 367 | ¹H NMR (500 MHz, CDCl₃) δ 7.83- |
| | | | ([M + H]⁺) | 7.73 (m, 1H), 7.69 (t, J = 1.8 Hz, 1H), |
| | | | | 7.65 (dt, J = 7.7, 1.5 Hz, 1H), 7.49 |
| | | | | (dt, J = 7.7, 1.5 Hz, 1H), 7.40 (t, J = |
| | | | | 7.7 Hz, 1H), 7.15 (s, 1H), 6.85 (s, |
| | | | | 1H), 3.69 (s, 3H), 3.67 (s, 2H), 3.52- |
| | | | | 3.09 (m, 5H), 2.30 (m, 6H), 1.27 (t, |
| | | | | J = 7.2 Hz, 3H) |
| 25 | | | ESIMS m/z 273 | ¹H NMR (500 MHz, CDCl₃) δ 7.49 |
| | | | ([M + H]⁺) | (s, 1H), 7.41 (s, 1H), 6.57 (s, 1H), |
| | | | | 3.94 (pd, J = 8.6, 1.1 Hz, 1H), 3.58- |
| | | | | 3.25 (m, 2H), 3.00 (s, 3H), 2.52 (s, |
| | | | | 3H), 2.42-2.32 (m, 2H), 2.27- |
| | | | | 2.18 (m, 5H), 2.02 (dp, J = 11.0, 8.8 |
| | | | | Hz, 1H), 1.90-1.81 (m, 1H), 1.21 |
| | | | | (t, J = 7.2 Hz, 3H); |
| | | | | ¹³C NMR (126 MHz, CDCl₃) δ |
| | | | | 203.30, 153.94, 151.77, 138.73, |
| | | | | 131.82, 129.45, 128.35, 122.41, |
| | | | | 47.80, 43.75, 31.98, 25.42, 21.86, |
| | | | | 17.94, 17.67, 14.33 |
| 26 | | | ESIMS m/z 301 | ¹H NMR (500 MHz, CDCl₃) δ 7.48 |
| | | | ([M + H]⁺) | (d, J = 15.0 Hz, 1H), 7.42 (s, 1H), |
| | | | | 6.57 (s, 1H), 3.55-3.27 (m, 2H), |
| | | | | 3.12 (tt, J = 11.4, 3.3 Hz, 1H), 3.01 (s, |
| | | | | 3H), 2.41 (s, 3H), 2.27 (s, 3H), 1.87- |
| | | | | 1.78 (m, 4H), 1.70 (dddt, J = 11.8, |
| | | | | 5.1, 3.3, 1.6 Hz, 1H), 1.49-1.40 (m, |
| | | | | 2H), 1.35 (qt, J = 12.6, 2.7 Hz, 2H), |
| | | | | 1.27 (dt, J = 12.4, 3.3 Hz, 1H), 1.21 |
| | | | | (t, J = 7.1 Hz, 3H); |
| | | | | ¹³C NMR (126 MHz, CDCl₃) δ |
| | | | | 207.10, 153.48, 151.78, 137.58, |
| | | | | 131.51, 130.57, 128.35, 122.21, |
| | | | | 47.86, 41.68, 31.96, 29.36, 26.08, |
| | | | | 25.93, 21.24, 17.68, 14.33 |
| 27 | | | ESIMS m/z 287 | ¹H NMR (500 MHz, CDCl₃) δ 7.51 |
| | | | ([M + H]⁺) | (s, 1H), 7.50-7.43 (m, 1H), 6.56 (s, |
| | | | | 1H), 3.56-3.26 (m, 2H), 3.02-2.98 |
| | | | | (m, 5H), 2.79 (p, J = 7.8 Hz, 1H), |
| | | | | 2.46 (s, 3H), 2.27 (s, 3H), 2.14 (dddq, |
| | | | | J= 12.2, 9.8, 5.8, 2.0 Hz, 2H), 1.95- |
| | | | | 1.80 (m, 2H), 1.76-1.66 (m, 2H), |
| | | | | 1.20 (t, J = 7.1 Hz, 3H); |
| | | | | ¹³C NMR (126 MHz, CDCl₃) δ |
| | | | | 202.25, 153.89, 151.76, 137.95, |
| | | | | 131.66, 131.25, 128.40, 122.28, |
| | | | | 47.94, 47.79, 41.62, 32.48, 28.67, |
| | | | | 21.70, 18.95, 17.64, 14.27 |
| 29 | | | ESIMS m/z 365 | ¹H NMR (500 MHz, CDCl₃) δ 7.58 |
| | | | ([M + H]⁺) | (s, 1H), 7.54-7.40 (m, 1H), 6.59 (s, |
| | | | | 1H), 3.57-3.30 (m, 2H), 3.24-3.19 |
| | | | | (m, 2H), 3.02 (s, 3H), 2.57-2.44 (m, |
| | | | | 5H), 2.28 (s, 3H), 1.22 (t, J = 7.2 Hz, |
| | | | | 3H); |
| | | | | ¹⁹F NMR (471 MHz, CDCl₃) |
| | | | | δ −85.52, −118.11 (t, J = 18.7 Hz) |
| 30 | | | ESIMS m/z 305 | ¹H NMR (500 MHz, CDCl₃) δ 7.64 |
| | | | ([M + H]⁺) | (d, J = 8.6 Hz, 1H), 7.55-7.38 (m, |
| | | | | 1H), 6.46 (d, J = 13.0 Hz, 1H), 3.58- |
| | | | | 3.29 (m, 2H), 3.03 (s, 3H), 2.94 (dd, |
| | | | | J = 7.1, 3.0 Hz, 2H), 2.36 (dq, J = 8.6, |
| | | | | 7.0 Hz, 1H), 2.23 (s, 3H), 1.85 (tdd, |
| | | | | J = 11.7, 7.7, 4.8 Hz, 2H), 1.62 (dtd, |
| | | | | J = 12.5, 6.6, 3.6 Hz, 2H), 1.58-1.47 |
| | | | | (m, 2H), 1.28-1.11 (m, 5H); |
| | | | | ¹⁹F NMR (471 MHz, CDCl₃) |
| | | | | δ −111.97 (d, J = 11.5 Hz) |
| 31 | | | ESIMS m/z 385 | ¹H NMR (500 MHz, CDCl₃) δ 7.59- |
| | | | ([M + H]⁺) | 7.46 (m, 1H), 7.45-7.38 (m, 2H), |
| | | | | 7.21 (s, 1H), 7.18 (t, J = 7.6 Hz, 1H), |
| | | | | 6.63 (s, 1H), 3.70 (d, J = 1.9 Hz, 5H), |
| | | | | 3.54-3.25 (m, 2H), 3.02 (s, 3H), |
| | | | | 2.51 (s, 3H), 2.18 (s, 3H), 1.22 (t, J = |
| | | | | 7.2 Hz, 3H); |
| | | | | ¹⁹F NMR (471 MHz, CDCl₃) |
| | | | | δ −116.73 (t, J = 6.9 Hz) |
| 32 | | | ESIMS m/z 361 | ¹H NMR (500 MHz, CDCl₃) δ 7.68 |
| | | | ([M + H]⁺) | (s, 1H), 7.55-7.42 (m, 1H), 7.15 (t, |
| | | | | J = 8.1 Hz, 1H), 7.10-7.05 (m, 2H), |
| | | | | 6.59 (s, 1H), 4.22 (d, J = 1.2 Hz, 2H), |
| | | | | 3.42 (d, J = 102.8 Hz, 2H), 3.02 (s, |
| | | | | 3H), 2.48 (s, 3H), 2.29 (s, 3H), 1.22 |
| | | | | (t, J = 7.1 Hz, 3H); |
| | | | | ¹⁹F NMR (471 MHz, CDCl₃) |
| | | | | δ −114.38 (t, J = 8.6 Hz) |
| 33 | | | ESIMS m/z 393 | ¹H NMR (500 MHz, CDCl₃) δ 7.67 |
| | | | ([M + H]⁺) | (s, 1H), 7.54-7.45 (m, 1H), 7.26 |
| | | | | (dddd, J = 15.8, 9.8, 7.4, 3.9 Hz, 4H), |
| | | | | 6.59 (s, 1H), 4.29 (s, 2H), 3.57-3.27 |
| | | | | (m, 2H), 3.01 (s, 3H), 2.48 (s, 3H), |
| | | | | 2.28 (s, 3H), 1.21 (t, J = 7.2 Hz, 3H); |
| | | | | ¹⁹F NMR (471 MHz, CDCl₃) δ −56.96 |
| 34 | | | ESIMS m/z 393 | ¹H NMR (500 MHz, CDCl₃) δ 7.64 |
| | | | ([M + H]⁺) | (s, 1H), 7.51 (s, 1H), 7.33 (t, J = 7.9 |
| | | | | Hz, 1H), 7.18 (dt, J = 7.7, 1.3 Hz, |
| | | | | 1H), 7.12-7.07 (m, 2H), 6.58 (s, |
| | | | | 1H), 4.24 (s, 2H), 3.54-3.32 (m, |
| | | | | 2H), 3.02 (s, 3H), 2.47 (s, 3H), 2.28 |
| | | | | (s, 3H), 1.22 (t, J = 7.2 Hz, 3H); |
| | | | | ¹⁹F NMR (471 MHz, CDCl₃) δ −57.68 |
| 35 | | | ESIMS m/z 317 | ¹H NMR (500 MHz, CDCl₃) δ 7.55 |
| | | | ([M + H]⁺) | (s, 1H), 7.44 (m, 1H), 6.30 (s, 1H), |
| | | | | 3.86 (s, 3H), 3.56-3.28 (m, 2H), |
| | | | | 3.01 (s, 3H), 2.97 (d, J = 7.1 Hz, 2H), |
| | | | | 2.32 (hept, J = 7.7 Hz, 1H), 2.18 (s, |
| | | | | 3H), 1.86-1.76 (m, 2H), 1.60 (tq, J = |
| | | | | 10.0, 5.8, 4.1 Hz, 2H), 1.52 (qt, J = |
| | | | | 7.5, 3.3 Hz, 2H), 1.22 (t, J = 7.1 Hz, |
| | | | | 3H), 1.15 (dq, J = 12.6, 7.9 Hz, 2H); |
| | | | | ¹³C NMR (126 MHz, CDCl₃) δ |
| | | | | 201.46, 158.36, 156.15, 151.76, |
| | | | | 131.99, 123.69, 122.12, 102.59, |
| | | | | 55.53, 49.90, 47.84, 36.30, 32.70, |
| | | | | 31.95, 25.06, 17.01, 14.31 |
| 36 | | | ESIMS m/z 395 | ¹H NMR (500 MHz, CDCl₃) δ 7.70 |
| | | | ([M + H]⁺) | (s, 1H), 7.56-7.38 (m, 3H), 7.19 (t, J = |
| | | | | 7.7 Hz, 1H), 6.60 (s, 1H), 4.32 (s, |
| | | | | 2H), 3.63-3.22 (m, 2H), 3.02 (s, |
| | | | | 3H), 2.49 (s, 3H), 2.30 (s, 3H), 1.22 |
| | | | | (t, J = 7.2 Hz, 3H); |
| | | | | ¹⁹F NMR (471 MHz, CDCl₃) δ −61.25 |
| | | | | (d, J = 13.0 Hz), −118.97 (th, J = 13.2, |
| | | | | 6.7 Hz) |
| 37 | | | ESIMS m/z 379 | ¹H NMR (500 MHz, CDCl₃) δ 7.68 |
| | | | ([M + H]⁺) | (s, 1H), 7.53-7.36 (m, 1H), 6.70- |
| | | | | 6.60 (m, 2H), 6.33 (s, 1H), 4.29 (s, |
| | | | | 2H), 3.92 (s, 3H), 3.60-3.29 (m, |
| | | | | 2H), 3.03 (s, 3H), 2.18 (s, 3H), 1.24 |
| | | | | (t, J = 7.1 Hz, 3H); |
| | | | | ¹⁹F NMR (471 MHz, CDCl₃) |
| | | | | δ −110.95 (p, J = 6.8 Hz), −111.65 (t, |
| | | | | J = 6.5 Hz) |
| 38 | | | ESIMS m/z 369 | ¹H NMR (400 MHz, CDCl₃) δ 7.81- |
| | | | ([M + H]⁺) | 7.74 (m, 1H), 7.48 (d, J = 6.9 Hz, |
| | | | | 2H), 7.34 (d, J = 8.0 Hz, 1H), 6.57 (s, |
| | | | | 1H), 4.17 (s, 1H), 3.72 (s, 1H), 3.14 |
| | | | | (s, 3H), 2.74 (d, J = 6.9 Hz, 2H), 2.45 |
| | | | | (d, J = 10.2 Hz, 4H), 1.93 (ddd, J = |
| | | | | 11.2, 6.9, 3.1 Hz, 1H), 1.78-1.68 (m, |
| | | | | 3H), 1.66 (s, 1H), 1.61 (s, 1H), 1.28 |
| | | | | (qt, J = 13.3, 3.9 Hz, 2H), 1.23-1.07 |
| | | | | (m, 1H), 0.99 (qd, J = 12.3, 2.9 Hz, |
| | | | | 2H) |
| 39 | | | ESIMS m/z 327 | ¹H NMR (400 MHz, CDCl₃) δ 7.49 |
| | | | ([M + H]⁺) | (s, 2H), 6.58 (s, 1H), 5.84 (ddt, J = |
| | | | | 15.9, 10.5, 5.3 Hz, 1H), 5.28-5.19 |
| | | | | (m, 2H), 4.10 (s, 1H), 3.00 (s, 3H), |
| | | | | 2.74 (d, J = 6.9 Hz, 2H), 2.47 (s, 3H), |
| | | | | 2.27 (s, 3H), 1.93 (th, J = 10.6, 3.4 |
| | | | | Hz, 1H), 1.78-1.69 (m, 3H), 1.66 (s, |
| | | | | 2H), 1.35-1.08 (m, 4H), 0.99 (qd, |
| | | | | J = 12.1, 2.9 Hz, 2H) |
| 40 | | | ESIMS m/z 369 | ¹H NMR (400 MHz, CDCl₃) δ 7.42 |
| | | | ([M + H]⁺) | (s, 1H), 7.18 (s, 1H), 6.97 (s, 1H), |
| | | | | 4.96 (s, 1H), 3.05 (s, 3H), 2.72 (d, J = |
| | | | | 6.8 Hz, 2H), 2.37 (s, 3H), 2.23 (s, |
| | | | | 3H), 1.92-1.80 (m, 4H), 1.74-1.68 |
| | | | | (m, 3H), 1.67 (s, 3H), 1.43 (s, 3H), |
| | | | | 1.25 (t, J = 7.1 Hz, 4H), 1.19-1.05 |
| | | | | (m, 2H), 0.98 (qd, J = 13.6, 12.7, 3.6 |
| | | | | Hz, 2H) |
| 41 | | | ESIMS m/z 327 | ¹H NMR (400 MHz, CDCl₃) δ 7.69 |
| | | | ([M + H]⁺) | (s, 1H), 7.50 (s, 1H), 6.58 (s, 1H), |
| | | | | 3.55-3.47 (m, 5H), 2.74 (d, J = 6.8 |
| | | | | Hz, 2H), 2.47 (s, 3H), 2.28 (s, 3H), |
| | | | | 2.01-1.86 (m, 3H), 1.79-1.59 (m, |
| | | | | 5H), 1.36-1.08 (m, 4H), 0.99 (qd, |
| | | | | J = 12.2, 2.9 Hz, 2H) |
| 42 | | | ESIMS m/z 341 | ¹H NMR (400 MHz, CDCl₃) δ 7.46 |
| | | | ([M + H]⁺) | (d, J = 25.6 Hz, 2H), 6.58 (s, 1H), |
| | | | | 3.74-3.12 (m, 1H), 2.74 (d, J = 6.8 |
| | | | | Hz, 2H), 2.48 (s, 3H), 2.26 (s, 3H), |
| | | | | 1.93 (ddh, J = 14.2, 6.9, 3.4 Hz, 1H), |
| | | | | 1.79-1.55 (m, 10H), 1.36-1.29 (m, |
| | | | | 1H), 1.25 (ddd, J = 8.7, 6.8, 4.1 Hz, |
| | | | | 2H), 1.24-1.07 (m, 2H), 1.06-0.91 |
| | | | | (m, 4H) |
| 43 | | | ESIMS m/z 343 | ¹H NMR (400 MHz, CDCl₃) δ 7.47 |
| | | | ([M + H]⁺) | (d, J = 12.9 Hz, 2H), 6.59 (s, 1H), |
| | | | | 3.74 (dd, J = 5.7, 4.1 Hz, 4H), 3.53 (s, |
| | | | | 5H), 2.74 (d, J = 6.8 Hz, 2H), 2.47 (s, |
| | | | | 3H), 2.25 (s, 3H), 1.93 (tp, J = 10.6, |
| | | | | 3.4 Hz, 2H), 1.78-1.60 (m, 3H), |
| | | | | 1.35-1.08 (m, 3H), 0.98 (qd, J = |
| | | | | 12.3, 2.8 Hz, 2H) |
| 44 | | | ESIMS m/z 363 | ¹H NMR (400 MHz, CDCl₃) δ 8.02 |
| | | | ([M + H]⁺) | (s, 1H), 7.52 (s, 1H), 7.37 (ddd, J = |
| | | | | 9.4, 7.4, 2.0 Hz, 2H), 7.20-7.10 (m, |
| | | | | 3H), 6.65 (s, 1H), 3.51 (s, 3H), 2.75 |
| | | | | (d, J = 6.8 Hz, 2H), 2.46 (d, J = 16.9 |
| | | | | Hz, 4H), 2.33 (s, 3H), 2.02-1.88 (m, |
| | | | | 1H), 1.79-1.60 (m, 4H), 1.36-1.21 |
| | | | | (m, 2H), 1.16 (tdd, J = 16.0, 13.9, 8.4 |
| | | | | Hz, 1H), 1.00 (qd, J = 12.2, 2.8 Hz, |
| | | | | 2H) |
| 45 | | | ESIMS m/z 359 | ¹H NMR (400 MHz, CDCl₃) δ 7.41 |
| | | | ([M + H]⁺) | (s, 1H), 7.17 (s, 1H), 7.09 (s, 1H), |
| | | | | 5.91 (ddt, J = 17.4, 10.3, 5.1 Hz, 1H), |
| | | | | 5.36-5.24 (m, 2H), 4.41 (d, J = 5.2 |
| | | | | Hz, 2H), 3.31 (s, 3H), 2.71 (d, J = 6.8 |
| | | | | Hz, 2H), 2.40 (s, 1H), 2.38 (s, 3H), |
| | | | | 1.93 (tp, J = 10.7, 3.4 Hz, 1H), 1.76- |
| | | | | 1.61 (m, 5H), 1.32 (dd, J = 13.2, 3.3 |
| | | | | Hz, 1H), 1.26 (dt, J = 11.2, 6.7 Hz, |
| | | | | 3H), 1.21-1.10 (m, 1H), 0.99 (td, |
| | | | | J = 11.8, 2.8 Hz, 2H) |
| 46 | | | ESIMS m/z 401 | ¹H NMR (400 MHz, CDCl₃) δ 7.66 |
| | | | ([M + H]⁺) | (s, 1H), 7.33 (s, 1H), 6.95 (s, 1H), |
| | | | | 4.74 (q, J = 8.8 Hz, 2H), 3.40 (s, 3H), |
| | | | | 2.71 (d, J= 6.8 Hz, 2H), 2.31 (s, 3H), |
| | | | | 2.21 (s, 3H), 2.02 (s, 1H), 1.92 (dddd, |
| | | | | J = 14.6, 11.3, 5.9, 3.4 Hz, 1H), 1.79- |
| | | | | 1.61 (m, 3H), 1.36-1.20 (m, 3H), |
| | | | | 1.23-1.09 (m, 1H), 0.98 (qd, J = |
| | | | | 13.4, 12.6, 3.6 Hz, 2H); |
| | | | | ¹⁹F NMR (376 MHz, CDCl₃) δ −69.29 |
| 47 | | | ESIMS m/z 361 | ¹H NMR (400 MHz, CDCl₃) δ 7.42 |
| | | | ([M + H]⁺) | (s, 1H), 7.20 (s, 1H), 6.97 (s, 1H), |
| | | | | 5.43 (s, 1H), 3.03 (s, 3H), 2.72 (d, J = |
| | | | | 6.8 Hz, 2H), 2.37 (s, 3H), 2.24 (s, |
| | | | | 3H), 2.02 (s, 3H), 1.92 (ddt, J = 11.0, |
| | | | | 7.2, 3.7 Hz, 1H), 1.76-1.61 (m, 6H), |
| | | | | 1.32 (d, J = 11.1 Hz, 1H), 1.27 (s, |
| | | | | 1H), 1.25 (d, J = 7.2 Hz, 3H), 0.98 (tt, |
| | | | | J = 11.8, 5.9 Hz, 2H); |
| | | | | ¹³C NMR (101 MHz, CDCl₃) δ |
| | | | | 203.94, 181.70, 171.12, 141.04, |
| | | | | 136.39, 136.10, 131.05, 130.84, |
| | | | | 129.56, 60.34, 51.81, 49.01, 34.28, |
| | | | | 33.31, 26.22, 26.13, 20.98, 20.87, |
| | | | | 19.46, 17.75, 14.14 |
| 48 | | | ESIMS m/z 401 | ¹H NMR (400 MHz, CDCl₃) δ 7.42 |
| | | | ([M + H]⁺) | (s, 1H), 7.18 (s, 1H), 6.97 (s, 1H), |
| | | | | 4.96 (s, 1H), 3.05 (s, 3H), 2.72 (d, J = |
| | | | | 6.8 Hz, 2H), 2.37 (s, 3H), 2.23 (s, |
| | | | | 3H), 2.02 (s, 3H), 1.98-1.80 (m, |
| | | | | 6H), 1.77-1.56 (m, 7H), 1.48-1.37 |
| | | | | (m, 1H), 1.31 (dt, J = 12.4, 3.3 Hz, |
| | | | | 1H), 1.14 (ddt, J = 10.4, 7.6, 4.4 Hz, |
| | | | | 1H), 1.01 (dd, J = 12.0, 3.0 Hz, 1H), |
| | | | | 0.95 (dd, J = 12.7, 3.5 Hz, 1H) |
| 49 | | | ESIMS m/z 359 | ¹H NMR (400 MHz, CDCl₃) δ 7.43 |
| | | | ([M + H]⁺) | (s, 1H), 7.32 (s, 1H), 6.89 (s, 1H), |
| | | | | 3.81 (d, J = 5.7 Hz, 4H), 2.72 (d, J = |
| | | | | 6.8 Hz, 2H), 2.39 (s, 3H), 2.25 (s, |
| | | | | 3H), 1.93 (dp, J = 11.2, 3.8 Hz, 1H), |
| | | | | 1.78-1.68 (m, 4H), 1.25 (m, 7H), |
| | | | | 1.16 (dd, J = 12.1, 3.5 Hz, 1H), 1.05- |
| | | | | 0.84 (m, 2H) |
| 50 | | | ESIMS m/z 373 | ¹H NMR (400 MHz, CDCl₃) δ 7.43 |
| | | | ([M + H]⁺) | (s, 1H), 7.32 (s, 1H), 6.89 (s, 1H), |
| | | | | 3.81 (d, J = 5.7 Hz, 4H), 2.72 (d, J = |
| | | | | 6.8 Hz, 2H), 2.39 (s, 3H), 2.25 (s, |
| | | | | 3H), 1.93 (dp, J = 11.2, 3.8 Hz, 1H), |
| | | | | 1.78-1.68 (m, 5H), 1.63 (s, 1H), |
| | | | | 1.25 (m, 7H), 1.16 (dd, J = 12.1, 3.5 |
| | | | | Hz, 1H), 1.05-0.84 (m, 2H) |
| 51 | | | ESIMS m/z 395 | ¹H NMR (400 MHz, CDCl₃) δ 7.54 |
| | | | ([M + H]⁺) | (dd, J = 8.4, 7.1 Hz, 2H), 7.47-7.31 |
| | | | | (m, 5H), 6.86 (s, 1H), 3.74 (s, 3H), |
| | | | | 2.70 (d, J = 6.8 Hz, 2H), 2.41 (s, 3H), |
| | | | | 2.12 (s, 3H), 2.01-1.84 (m, 1H), |
| | | | | 1.75-1.67 (m, 3H), 1.66 (s, 2H), |
| | | | | 1.34-1.27 (m, 1H), 1.20-1.06 (m, |
| | | | | 1H), 1.03-0.82 (m, 3H) |
| 52 | | | ESIMS m/z 333 | ¹H NMR (400 MHz, CDCl₃) δ 7.47 |
| | | | ([M + H]⁺) | (s, 1H), 7.04 (s, 1H), 6.80 (s, 1H), |
| | | | | 3.34 (s, 6H), 2.74 (d, J = 6.8 Hz, 2H), |
| | | | | 2.44 (s, 3H), 2.28 (s, 3H), 1.95 (ddt, J = |
| | | | | 11.3, 7.4, 3.4 Hz, 1H), 1.76 (d, J = |
| | | | | 3.8 Hz, 1H), 1.70 (d, J = 16.0 Hz, |
| | | | | 4H), 1.37-1.23 (m, 2H), 1.27-1.11 |
| | | | | (m, 1H), 1.06-0.92 (m, 2H) |
| 53 | | | ESIMS m/z 409 | ¹H NMR (400 MHz, CDCl₃) δ 7.45- |
| | | | ([M + H]⁺) | 7.38 (m, 2H), 7.38-7.30 (m, 3H), |
| | | | | 7.12 (s, 1H), 6.81 (s, 1H), 5.07 (s, |
| | | | | 2H), 3.34 (s, 3H), 2.72 (d, J = 6.8 Hz, |
| | | | | 2H), 2.42 (s, 2H), 2.12 (s, 3H), 1.93 |
| | | | | (ddt, J = 10.8, 7.0, 3.5 Hz, 1H), 1.69 |
| | | | | (q, J = 13.8, 13.4 Hz, 4H), 1.56 (s, |
| | | | | 3H), 1.36-1.22 (m, 2H), 1.22-1.08 |
| | | | | (m, 1H), 1.04-0.91 (m, 2H) |
| 54 | | | ESIMS m/z 359 | ¹H NMR (400 MHz, CDCl₃) δ 7.80 |
| | | | ([M + H]⁺) | (s, 1H), 7.49 (s, 1H), 7.36 (s, 1H), |
| | | | | 3.46 (s, 3H), 2.84-2.72 (m, 3H), |
| | | | | 2.46 (s, 3H), 2.30 (s, 3H), 1.97 (ddh, |
| | | | | J = 10.8, 7.0, 3.4 Hz, 1H), 1.76 (s, 1H), |
| | | | | 1.70 (d, J = 17.2 Hz, 4H), 1.37-1.23 |
| | | | | (m, 2H), 1.23-0.92 (m, 7H); |
| | | | | ¹³C NMR (101 MHz, CDCl₃) d |
| | | | | 201.81, 181.06, 138.36, 134.98, |
| | | | | 134.79, 129.22, 128.92, 47.32, 39.22, |
| | | | | 32.47, 31.58, 29.63, 24.47, 24.35, |
| | | | | 19.18, 16.18, 7.54, −1.82 |
| 55 | | | ESIMS m/z 361 | ¹H NMR (400 MHz, CDCl₃) δ 7.47 |
| | | | ([M + H]⁺) | (s, 1H), 7.17 (s, 1H), 6.77 (s, 1H), |
| | | | | 3.79 (q, J = 7.1 Hz, 4H), 2.74 (d, J = |
| | | | | 6.8 Hz, 2H), 2.44 (s, 3H), 2.28 (s, |
| | | | | 3H), 2.04 (s, 3H), 1.78-1.58 (m, |
| | | | | 6H), 1.26 (m, 5H), 1.20-1.11 (m, |
| | | | | 1H), 1.06-0.92 (m, 2H) |
| 56 | | | ESIMS m/z 377 | ¹H NMR (400 MHz, CDCl₃) δ 7.46- |
| | | | ([M + H]⁺) | 7.38 (m, 3H), 7.35 (dd, J = 7.1, 4.0 |
| | | | | Hz, 4H), 7.12 (s, 1H), 6.81 (s, 1H), |
| | | | | 5.07 (s, 2H), 3.34 (s, 4H), 2.72 (d, J = |
| | | | | 6.8 Hz, 2H), 2.42 (s, 3H), 2.12 (s, |
| | | | | 3H), 1.94 (td, J = 7.3, 3.6 Hz, 1H), |
| | | | | 1.73 (d, J = 14.3 Hz, 3H), 1.33-1.24 |
| | | | | (m, 2H), 1.19-1.11 (m, 1H), 1.05- |
| | | | | 0.91 (m, 2H) |
| 57 | | | ESIMS m/z 301 | ¹H NMR (400 MHz, CDCl₃) δ 7.50 |
| | | | ([M + H]⁺) | (s, 1H), 7.45 (s, 1H), 6.57 (s, 1H), |
| | | | | 3.03 (s, 6H), 2.74 (d, J = 6.8 Hz, 2H), |
| | | | | 2.47 (s, 3H), 2.27 (s, 3H), 1.94 (ddp, |
| | | | | J = 11.2, 7.4, 3.4 Hz, 1H), 1.74 (d, J = |
| | | | | 11.4 Hz, 2H), 1.66 (s, 2H), 1.35- |
| | | | | 1.10 (m, 4H), 0.99 (qd, J = 12.1, 2.9 |
| | | | | Hz, 2H) |
| 58 | | | ESIMS m/z 329 | ¹H NMR (400 MHz, CDCl₃) δ 7.50 |
| | | | ([M + H]⁺) | (s, 1H), 7.45 (s, 1H), 6.56 (s, 1H), |
| | | | | 3.49 (s, 2H), 3.31 (s, 2H), 2.74 (d, J = |
| | | | | 6.9 Hz, 2H), 2.48 (s, 3H), 2.27 (s, |
| | | | | 3H), 2.15 (s, 1H), 1.93 (ddp, J = 10.7, |
| | | | | 7.0, 3.6 Hz, 1H), 1.78-1.60 (m, 3H), |
| | | | | 1.35-1.20 (m, 7H), 1.23-1.10 (m, |
| | | | | 2H), 0.99 (qd, J = 12.1, 2.9 Hz, 3H); |
| | | | | ¹³C NMR (101 MHz, CDCl₃) δ |
| | | | | 202.87, 154.08, 151.07, 137.93, |
| | | | | 131.63, 131.61, 128.48, 122.20, |
| | | | | 48.56, 34.81, 33.47, 26.38, 26.25, |
| | | | | 21.72, 17.75 |
| 59 | | | ESIMS m/z 329 | ¹H NMR (400 MHz, CDCl₃) δ 7.47 |
| | | | ([M + H]⁺) | (s, 1H), 6.57 (s, 1H), 6.45 (s, 1H), |
| | | | | 3.95 (s, 2H), 2.93 (s, 3H), 2.47 (d, J = |
| | | | | 6.2 Hz, 6H), 2.27 (s, 3H), 2.15 (s, |
| | | | | 3H), 1.99-1.88 (m, 2H), 1.79-1.70 |
| | | | | (m, 3H), 1.65 (s, 2H), 1.22-1.10 (m, |
| | | | | 1H), 0.98 (qt, J = 12.0, 2.9 Hz, 4H) |
| 60 | | | ESIMS m/z 327 | ¹H NMR (400 MHz, CDCl₃) δ 7.64 |
| | | | ([M + H]⁺) | (s, 1H), 6.57 (s, 1H), 6.46 (s, 1H), |
| | | | | 3.05 (s, 3H), 2.74 (d, J = 6.9 Hz, 2H), |
| | | | | 2.26 (s, 3H), 2.16 (s, 3H), 1.93 (ddh, |
| | | | | J = 14.5, 7.0, 3.4 Hz, 2H), 1.27 (dtt, J = |
| | | | | 13.0, 6.8, 3.3 Hz, 4H), 1.21-1.10 (m, |
| | | | | 2H), 1.05-0.91 (m, 5H), 0.85-0.68 |
| | | | | (m, 3H) |
| 61 | | | ESIMS m/z 347 | ¹H NMR (400 MHz, CDCl₃) δ 7.46 |
| | | | ([M + H]⁺) | (s, 1H), 7.07 (s, 1H), 6.82 (s, 1H), |
| | | | | 3.88 (q, J = 7.1 Hz, 2H), 3.25 (s, 3H), |
| | | | | 2.74 (d, J = 6.8 Hz, 2H), 2.43 (s, 3H), |
| | | | | 2.27 (s, 3H), 2.17 (s, 6H), 1.95 (qd, |
| | | | | J = 7.3, 6.5, 2.9 Hz, 1H), 1.78-1.62 |
| | | | | (m, 5H), 1.06-0.92 (m, 2H) |
| 62 | | | ESIMS m/z 337 | ¹H NMR (500 MHz, CDCl₃) δ 7.67 |
| | | | ([M + H]⁺) | (s, 1H), 7.54-7.36 (m, 1H), 7.02- |
| | | | | 6.97 (m, 2H), 6.94 (dd, J = 8.0, 1.6 |
| | | | | Hz, 1H), 6.58 (s, 1H), 4.19 (s, 2H), |
| | | | | 3.41 (m, 2H), 3.00 (s, 3H), 2.46 (s, |
| | | | | 3H), 2.28 (s, 6H), 2.20 (s, 3H), 1.21 |
| | | | | (t, J = 7.2 Hz, 3H); |
| | | | | ¹³C NMR (126 MHz, CDCl₃) δ |
| | | | | 199.61, 154.18, 151.82, 138.65, |
| | | | | 136.67, 136.39, 131.75, 131.41, |
| | | | | 131.12, 130.86, 130.42, 128.56, |
| | | | | 126.70, 122.42, 47.92, 45.15, 32.03, |
| | | | | 21.88, 21.06, 19.77, 17.75, 14.40 |
| 63 | | | ESIMS m/z 377 | ¹H NMR (500 MHz, CDCl₃) δ 7.66 |
| | | | ([M + H]⁺) | (s, 1H), 7.56 (d, J = 8.0 Hz, 2H), 7.53- |
| | | | | 7.38 (m, 1H), 7.35 (d, J = 8.0 Hz, |
| | | | | 2H), 6.59 (s, 1H), 4.28 (s, 2H), 3.61- |
| | | | | 3.23 (m, 2H), 3.02 (s, 3H), 2.47 (s, |
| | | | | 3H), 2.29 (s, 3H), 1.22 (t, J = 7.1 Hz, |
| | | | | 3H); |
| | | | | ¹⁹F NMR (471 MHz, CDCl₃) δ −62.41 |
| 64 | | | ESIMS m/z 351 | ¹H NMR (500 MHz, CDCl₃) δ 7.66 |
| | | | ([M + H]⁺) | (s, 1H), 7.48 (s, 1H), 7.18-7.08 (m, |
| | | | | 4H), 6.56 (s, 1H), 4.17 (s, 2H), 3.31 |
| | | | | (s, 2H), 3.00 (s, 3H), 2.54 (dd, J = |
| | | | | 8.6, 6.7 Hz, 2H), 2.46 (s, 3H), 2.27 (d, |
| | | | | J = 4.0 Hz, 3H), 1.62 (q, J = 7.5 Hz, |
| | | | | 2H), 1.20 (t, J = 7.2 Hz, 3H), 0.93 (t, |
| | | | | J = 7.4 Hz, 3H); |
| | | | | ¹³C NMR (126 MHz, CDCl₃) δ |
| | | | | 199.82, 154.17, 151.83, 140.87, |
| | | | | 138.91, 132.74, 132.14, 130.60, |
| | | | | 129.42, 128.61, 128.52, 122.46, |
| | | | | 47.92, 47.13, 37.74, 32.05, 24.54, |
| | | | | 21.96, 17.74, 14.34, 13,93 |
| 65 | | | ESIMS m/z 409 | ¹H NMR (500 MHz, CDCl₃) δ 7.65 |
| | | | ([M + H]⁺) | (s, 1H), 7.60 (d, J = 8.0 Hz, 2H), 7.54- |
| | | | | 7.40 (m, 1H), 7.32-7.28 (m, 2H), |
| | | | | 6.59 (s, 1H), 4.26 (s, 2H), 3.62-3.23 |
| | | | | (m, 2H), 3.02 (s, 3H), 2.47 (s, 3H), |
| | | | | 2.28 (s, 3H), 1.22 (t, J = 7.2 Hz, 3H); |
| | | | | ¹³C NMR (126 MHz, CDCl₃) δ |
| | | | | 198.26, 154.59, 151.87, 139.27, |
| | | | | 138.94, 136.42, 132.11, 130.89, |
| | | | | 129.95, 128.74, 122.53, 122.31, |
| | | | | 47.91, 46.88, 37.60, 32.01, 22.06, |
| | | | | 17.74, 14.35; ¹⁹F NMR (471 MHz, |
| | | | | CDCl₃) δ −42.79 |
| 66 | | | ESIMS m/z 351 | ¹H NMR (500 MHz, CDCl₃) δ 7.67 |
| | | | ([M + H]⁺) | (s, 1H), 7.47 (m, 1H), 7.17 (s, 4H), |
| | | | | 6.57 (s, 1H), 4.18 (s, 2H), 3.59-3.24 |
| | | | | (m, 2H), 3.01 (s, 3H), 2.87 (hept, J = |
| | | | | 6.9 Hz, 1H), 2.47 (s, 3H), 2.27 (s, |
| | | | | 3H), 1.22 (m, 9H); |
| | | | | ¹³C NMR (126 MHz, CDCl₃) δ |
| | | | | 199.74, 154.19, 151.84, 146.99, |
| | | | | 138.96, 132.83, 132.15, 130.56, |
| | | | | 129.50, 128.50, 126.58, 122.45, |
| | | | | 47.89, 47.02, 33.75, 31.95, 24.02, |
| | | | | 21.99, 17.72, 14.32 |
| 67 | | | ESIMS m/z 365 | ¹H NMR (500 MHz, CDCl₃) δ 7.68 |
| | | | ([M + H]⁺) | (s, 1H), 7.54-7.42 (m, 1H), 7.35- |
| | | | | 7.31 (m, 2H), 7.20-7.16 (m, 2H), |
| | | | | 6.57 (s, 1H), 4.19 (s, 2H), 3.46-3.24 |
| | | | | (m, 2H), 3.02 (s, 3H), 2.47 (s, 3H), |
| | | | | 2.28 (s, 3H), 1.30 (s, 9H), 1.22 (t, J = |
| | | | | 7.1 Hz, 3H); |
| | | | | ¹³C NMR (126 MHz, CDCl₃) δ |
| | | | | 199.67, 154.19, 151.81, 149.20, |
| | | | | 138.97, 132.46, 132.15, 130.52, |
| | | | | 129.23, 128.49, 125.42, 122.42, |
| | | | | 47.88, 46.85, 34.40, 31.93, 31.37, |
| | | | | 22.00, 17.70, 14.30 |
| 68 | | | ESIMS m/z 327 | ¹H NMR (500 MHz, CDCl₃) δ 7.64 |
| | | | ([M + H]⁺) | (s, 1H), 7.55-7.42 (m, 1H), 7.21- |
| | | | | 7.15 (m, 2H), 7.01-6.95 (m, 2H), |
| | | | | 6.58 (s, 1H), 4.18 (s, 2H), 3.56-3.27 |
| | | | | (m, 2H), 3.01 (d, J = 3.2 Hz, 3H), |
| | | | | 2.46 (s, 3H), 2.28 (s, 3H), 1.21 (t, J = |
| | | | | 7.1 Hz, 3H); |
| | | | | ¹⁹F NMR (471 MHz, CDCl₃) |
| | | | | δ −116.67 (tt, J = 9.4, 5.3 Hz) |
| 69 | | | ESIMS m/z 343 | ¹H NMR (500 MHz, CDCl₃) δ 7.63 |
| | | | ([M + H]⁺) | (s, 1H), 7.53-7.43 (m, 1H), 7.27 (d, |
| | | | | J = 8.1 Hz, 2H), 7.16 (d, J = 8.1 Hz, |
| | | | | 2H), 6.58 (s, 1H), 4.18 (s, 2H), 3.62- |
| | | | | 3.25 (m, 2H), 3.02 (s, 3H), 2.46 (s, |
| | | | | 3H), 2.27 (s, 3H), 1.22 (t, J = 7.1 Hz, |
| | | | | 3H); |
| | | | | ¹³C NMR (126 MHz, CDCl₃) δ |
| | | | | 198.85, 154.44, 151.85, 139.11, |
| | | | | 134.06, 132.48, 132.04, 130.99, |
| | | | | 130.11, 128.65, 128.60, 122.49, |
| | | | | 47.97, 46.65, 32.02, 21.99, 17.73, |
| | | | | 14.36 |
| 70 | | | ESIMS m/z 337 | ¹H NMR (500 MHz, CDCl₃) δ 7.66 |
| | | | ([M + H]⁺) | (s, 1H), 7.49 (s, 1H), 7.21 (t, J = 7.5 |
| | | | | Hz, 1H), 7.08 (s, 1H), 7.07-7.03 (m, |
| | | | | 2H), 6.56 (s, 1H), 4.18 (s, 2H), 3.31 |
| | | | | (s, 2H), 3.01 (s, 3H), 2.62 (q, J = 7.6 |
| | | | | Hz, 2H), 2.46 (s, 3H), 2.27 (s, 3H), |
| | | | | 1.25-1.19 (m, 6H); |
| | | | | ¹³C NMR (126 MHz, CDCl₃) δ |
| | | | | 199.75, 154.18, 151.83, 144.39, |
| | | | | 138.89, 135.53, 132.15, 130.61, |
| | | | | 129.17, 128.49, 128.44, 126.86, |
| | | | | 126.11, 122.41, 47.84, 47.58, 31.98, |
| | | | | 28.81, 21.92, 17.72, 15.55, 14.32 |
| 71 | | | ESIMS m/z 349 | ¹H NMR (500 MHz, CDCl₃) δ 7.67 |
| | | | ([M + H]⁺) | (s, 1H), 7.49 (s, 1H), 7.15 (d, J = 7.2 |
| | | | | Hz, 1H), 7.10 (s, 1H), 6.99 (dd, J = |
| | | | | 7.7, 1.6 Hz, 1H), 6.56 (s, 1H), 4.17 (s, |
| | | | | 2H), 3.62-3.20 (m, 2H), 3.01 (s, |
| | | | | 3H), 2.86 (q, J = 7.5 Hz, 4H), 2.47 (s, |
| | | | | 3H), 2.27 (s, 3H), 2.06-2.01 (m, |
| | | | | 2H), 1.21 (t, J = 7.1 Hz, 3H); |
| | | | | ¹³C NMR (126 MHz, CDCl₃) δ |
| | | | | 199.95, 154.19, 151.80, 144.56, |
| | | | | 142.50, 138.94, 133.25, 132.16, |
| | | | | 130.59, 128.50, 127.39, 125.61, |
| | | | | 124.34, 122.43, 47.91, 47.30, 32.85, |
| | | | | 32.56, 32.00, 25.51, 22.01, 17.74, |
| | | | | 14.24 |
| 72 | | | ESIMS m/z 331 | ¹H NMR (400 MHz, CDCl₃) δ 7.53 |
| | | | ([M + H]⁺) | (d, J = 0.8 Hz, 1H), 7.47 (s, 1H), 6.30 |
| | | | | (s, 1H), 3.86 (s, 3H), 3.52 (s, 1H), |
| | | | | 3.02 (s, 3H), 2.81 (d, J = 6.8 Hz, 2H), |
| | | | | 2.18 (s, 3H), 1.90 (dtd, J = 14.7, 7.4, |
| | | | | 3.4 Hz, 1H), 1.73 (d, J = 2.5 Hz, 1H), |
| | | | | 1.70 (s, 2H), 1.75-1.66 (m, 1H), |
| | | | | 1.64 (s, 2H), 1.35-1.18 (m, 5H), |
| | | | | 1.14 (dd, J = 12.1, 3.5 Hz, 1H), 0.97 |
| | | | | (qd, J = 12.2, 2.8 Hz, 2H) |
| 73 | | | ESIMS m/z 287 | ¹H NMR (500 MHz, CDCl₃) δ 7.50 |
| | | | ([M + H]⁺) | (s, 1H), 7.45 (s, 1H), 6.57 (s, 1H), |
| | | | | 3.03 (s, 6H), 2.90 (d, J = 7.2 Hz, 2H), |
| | | | | 2.47 (s, 3H), 2.37-2.29 (m, 1H), |
| | | | | 2.27 (s, 3H), 1.85 (ddt, J = 16.1, 11.9, |
| | | | | 4.7 Hz, 2H), 1.62 (qd, J = 9.7, 8.2, 3.6 |
| | | | | Hz, 2H), 1.54 (dq, J = 9.0, 4.2, 3.8 |
| | | | | Hz, 2H), 1.21-1.11 (m, 2H); |
| | | | | ¹³C NMR (126 MHz, CDCl₃) δ |
| | | | | 203.17, 153.59, 152.29, 137.84, |
| | | | | 131.69, 131.54, 128.36, 122.34, |
| | | | | 47.19, 40.14, 36.51, 34.29, 32.71, |
| | | | | 25.02, 21.64, 17.63 |
| 74 | | | ESIMS m/z 317 | ¹H NMR (500 MHz, CDCl₃) δ 7.58 |
| | | | ([M + H]⁺) | (d, J = 62.6 Hz, 1H), 7.17 (s, 1H), |
| | | | | 6.65 (s, 1H), 3.87 (s, 3H), 3.64-3.25 |
| | | | | (m, 2H), 3.03 (d, J = 24.7 Hz, 3H), |
| | | | | 2.90 (d, J = 7.1 Hz, 2H), 2.44 (s, 3H), |
| | | | | 2.35 (hept, J = 7.5 Hz, 1H), 1.92- |
| | | | | 1.80 (m, 2H), 1.63 (qd, J = 9.5, 8.0, |
| | | | | 4.1 Hz, 2H), 1.55 (qd, J = 8.0, 7.2, 4.2 |
| | | | | Hz, 2H), 1.27-1.11 (m, 5H); |
| | | | | ¹³C NMR (126 MHz, CDCl₃) δ |
| | | | | 202.92, 153.56, 149.83, 144.78, |
| | | | | 132.39, 131.89, 124.64, 112.53, |
| | | | | 56.21, 48.14, 47.41, 36.47, 32.74, |
| | | | | 32.21, 25.01, 21.10, 14.30 |
| 75 | | | ESIMS m/z 287 | ¹H NMR (500 MHz, CDCl₃) δ 7.77 |
| | | | ([M + H]⁺) | (d, J = 2.1 Hz, 1H), 7.72 (dd, J = 8.1, |
| | | | | 2.2 Hz, 1H), 7.47 (d, J = 42.9 Hz, |
| | | | | 1H), 6.76 (d, J = 8.2 Hz, 1H), 3.59- |
| | | | | 3.27 (m, 2H), 3.02 (s, 3H), 2.92 (d, |
| | | | | J = 7.1 Hz, 2H), 2.41-2.33 (m, 1H), |
| | | | | 2.30 (s, 3H), 1.91-1.81 (m, 2H), |
| | | | | 1.64 (qdt, J = 9.9, 6.4, 3.2 Hz, 2H), |
| | | | | 1.59-1.49 (m, 2H), 1.25-1.13 (m, |
| | | | | 5H); |
| | | | | ¹³C NMR (126 MHz, CDCl₃) δ |
| | | | | 199.79, 155.58, 151.86, 131.64, |
| | | | | 131.44, 130.24, 127.38, 118.28, |
| | | | | 47.88, 44.45, 36.47, 32.77, 32.00, |
| | | | | 25.01, 18.16, 14.29 |
| 76 | | | ESIMS m/z 349 | ¹H NMR (500 MHz, CDCl₃) δ 7.71 |
| | | | ([M + H]⁺) | (s, 1H), 7.47 (s, 1H), 6.72-6.64 (m, |
| | | | | 2H), 6.60 (s, 1H), 4.27 (s, 2H), 3.05 |
| | | | | (s, 6H), 2.49 (s, 3H), 2.30 (s, 3H); |
| | | | | ¹⁹F NMR (471 MHz, CDCl₃) |
| | | | | δ −110.12-−110.30 (m), −111.70 (t, |
| | | | | J = 6.5 Hz) |
| 77 | | | ESIMS m/z 315 | H NMR (500 MHz, CDCl₃) δ 7.51 |
| | | | ([M + H]⁺) | (s, 1H), 7.45 (s, 1H), 6.56 (s, 1H), |
| | | | | 3.62-3.21 (m, 4H), 2.90 (d, J = 7.2 |
| | | | | Hz, 2H), 2.48 (s, 3H), 2.37-2.29 (m, |
| | | | | 1H), 2.26 (s, 3H), 1.90-1.78 (m, |
| | | | | 2H), 1.62 (ddq, J = 12.7, 6.7, 3.0 Hz, |
| | | | | 2H), 1.54 (dddd, J = 14.3, 9.3, 4.5, |
| | | | | 2.2 Hz, 2H), 1.23 (t, J = 7.1 Hz, 6H), |
| | | | | 1.20-1.11 (m, 2H); |
| | | | | ¹³C NMR (126 MHz, CDCl₃) δ |
| | | | | 203.12, 154.03, 151.09, 137.92, |
| | | | | 131.54, 131.48, 128.49, 122.21, |
| | | | | 47.17, 45.64, 39.62, 36.55, 32.71, |
| | | | | 25.02, 21.66, 17.72, 15.10, 12.30 |
| 78 | | | ESIMS m/z 377 | ¹H NMR (500 MHz, CDCl₃) δ 7.71 |
| | | | ([M + H]⁺) | (s, 1H), 7.47 (s, 1H), 6.71-6.63 (m, |
| | | | | 2H), 6.59 (s, 1H), 4.26 (s, 2H), 3.59- |
| | | | | 3.26 (m, 4H), 2.49 (s, 3H), 2.30 (s, |
| | | | | 3H), 1.24 (t, J = 7.2 Hz, 6H); |
| | | | | ¹⁹F NMR (471 MHz, CDCl₃) |
| | | | | δ −110.17-−110.31 (m), −111.69 (t, |
| | | | | J = 6.5 Hz) |
| 79 | | | ESIMS m/z 377 | ¹H NMR (500 MHz, CDCl₃) δ 7.72 |
| | | | ([M + H]⁺) | (s, 1H), 7.59 (s, 1H), 6.73-6.63 (m, |
| | | | | 2H), 6.60 (s, 1H), 4.27 (s, 2H), 3.70 |
| | | | | (d, J = 14.5 Hz, 1H), 2.95 (s, 3H), |
| | | | | 2.51 (s, 3H), 2.33 (s, 3H), 1.27 (s, |
| | | | | 6H); |
| | | | | ¹⁹F NMR (471 MHz, CDCl₃) |
| | | | | δ −110.23 (tt, J = 9.5, 5.3 Hz), −111.69 (t, |
| | | | | J = 6.6 Hz) |
| 80 | | | ESIMS m/z 327 | ¹H NMR (500 MHz, CDCl₃) δ 7.50 |
| | | | ([M + H]⁺) | (s, 1H), 7.43 (s, 1H), 6.58 (s, 1H), |
| | | | | 3.48 (s, 4H), 2.90 (d, J = 7.2 Hz, 2H), |
| | | | | 2.47 (s, 3H), 2.33 (dtd, J = 14.4, 7.3, |
| | | | | 1.3 Hz, 1H), 2.26 (s, 3H), 1.88-1.80 |
| | | | | (m, 2H), 1.70 (dtd, J = 10.9, 5.3, 2.1 |
| | | | | Hz, 2H), 1.62 (tq, J = 8.9, 5.9, 4.6 Hz, |
| | | | | 6H), 1.53 (qdd, J = 10.0, 4.2, 2.5 Hz, |
| | | | | 2H), 1.21-1.11 (m, 2H); |
| | | | | ¹³C NMR (126 MHz, CDCl₃) δ |
| | | | | 203.14, 153.69, 151.42, 137.88, |
| | | | | 131.59, 131.55, 128.40, 122.11, |
| | | | | 50.15, 47.17, 43.26, 36.52, 32.71, |
| | | | | 25.02, 24.78, 21.66, 17.65 |
| 81 | | | ESIMS m/z 389 | ¹H NMR (500 MHz, CDCl₃) δ 7.71 |
| | | | ([M + H]⁺) | (s, 1H), 7.46 (s, 1H), 6.71-6.64 (m, |
| | | | | 2H), 6.61 (s, 1H), 4.26 (s, 2H), 3.75- |
| | | | | 3.26 (m, 4H), 2.49 (s, 3H), 2.30 (s, |
| | | | | 3H), 1.70 (tt, J = 6.8, 2.4 Hz, 2H), |
| | | | | 1.66-1.59 (m, 4H); |
| | | | | ¹⁹F NMR (471 MHz, CDCl₃) |
| | | | | δ −110.22 (ddd, J = 14.5, 9.6, 5.6 |
| | | | | Hz), −111.69 (t, J = 6.5 Hz) |
| 82 | | | ESIMS m/z 315 | ¹H NMR (500 MHz, CDCl₃) δ 7.57 |
| | | | ([M + H]⁺) | (s, 1H), 7.51 (s, 1H), 6.57 (s, 1H), |
| | | | | 3.67 (s, 1H), 2.97-2.87 (m, 5H), |
| | | | | 2.48 (s, 3H), 2.37-2.29 (m, 1H), |
| | | | | 2.27 (s, 3H), 1.88-1.80 (m, 2H), |
| | | | | 1.62 (ddp, J = 9.6, 6.7, 3.1 Hz, 2H), |
| | | | | 1.58-1.48 (m, 2H), 1.25 (d, J = 6.7 |
| | | | | Hz, 6H), 1.21-1.12 (m, 2H); |
| | | | | ¹³C NMR (126 MHz, CDCl₃) δ |
| | | | | 203.12, 154.00, 151.59, 137.89, |
| | | | | 131.56, 131.52, 128.38, 122.29, |
| | | | | 53.13, 47.17, 36.53, 32.71, 28.03, |
| | | | | 25.02, 21.66, 21.05, 17.67 |
| 83 | | | ESIMS m/z 375 | ¹H NMR (500 MHz, CDCl₃) δ 7.71 |
| | | | ([M + H]⁺) | (s, 1H), 7.55-7.37 (m, 1H), 7.28 (td, |
| | | | | J= 7.7, 1.9 Hz, 1H), 7.24 (dd, J = 7.9, |
| | | | | 2.1 Hz, 1H), 7.18 (td, J = 7.4, 1.2 Hz, |
| | | | | 1H), 7.14 (dd, J = 8.1, 1.2 Hz, 1H), |
| | | | | 6.63-6.29 (m, 2H), 4.30 (s, 2H), |
| | | | | 3.43 (d, J = 98.9 Hz, 2H), 3.02 (s, |
| | | | | 3H), 2.47 (s, 3H), 2.29 (s, 3H), 1.22 |
| | | | | (t, J = 7.1 Hz, 3H); |
| | | | | ¹⁹F NMR (471 MHz, CDCl₃) δ −79.31 |
| | | | | (d, J = 74.5 Hz) |
| 84 | | | ESIMS m/z 375 | ¹H NMR (500 MHz, CDCl₃) δ 7.64 |
| | | | ([M + H]⁺) | (s, 1H), 7.54-7.45 (m, 1H), 7.30 (t, |
| | | | | J = 7.9 Hz, 1H), 7.09 (d, J = 7.7 Hz, |
| | | | | 1H), 7.02-6.96 (m, 2H), 6.65-6.33 |
| | | | | (m, 2H), 4.22 (s, 2H), 3.46-3.23 (m, |
| | | | | 2H), 3.02 (s, 3H), 2.47 (s, 3H), 2.28 |
| | | | | (s, 3H), 1.23-1.19 (m, 3H); |
| | | | | ¹⁹F NMR (471 MHz, CDCl₃) δ −80.38 |
| | | | | (d, J = 74.5 Hz) |
| 85 | | | ESIMS m/z 375 | ¹H NMR (500 MHz, CDCl₃) δ 7.65 |
| | | | ([M + H]⁺) | (s, 1H), 7.50 (s, 1H), 7.23 (d, J = 8.2 |
| | | | | Hz, 2H), 7.06 (d, J = 8.3 Hz, 2H), |
| | | | | 6.64-6.30 (m, 2H), 4.21 (s, 2H), |
| | | | | 3.42-3.31 (m, 2H), 3.02 (s, 3H), |
| | | | | 2.46 (s, 3H), 2.28 (s, 3H), 1.22 (t, J = |
| | | | | 7.2 Hz, 3H); |
| | | | | ¹⁹F NMR (471 MHz, CDCl₃) δ −80.40 |
| | | | | (d, J = 74.3 Hz) |
| 86 | | | ESIMS m/z 377 | ¹H NMR (500 MHz, CDCl₃) δ 7.71 |
| | | | ([M + H]⁺) | (s, 1H), 6.68 (t, J = 8.0 Hz, 2H), 6.48 |
| | | | | (s, 1H), 4.27 (s, 2H), 3.46 (q, J = 6.5, |
| | | | | 5.7 Hz, 2H), 3.02 (s, 3H), 2.47 (s, |
| | | | | 3H), 2.11 (s, 3H), 1.81 (s, 3H), 1.19 |
| | | | | (t, J = 7.0 Hz, 3H); |
| | | | | ¹⁹F NMR (471 MHz, CDCl₃) |
| | | | | δ −110.27 (t, J = 8.0 Hz), −111.71 (t, |
| | | | | J = 7.0 Hz) |
| 87 | | | ESIMS m/z 391 | ¹H NMR (500 MHz, CDCl₃) δ 7.72 |
| | | | ([M + H]⁺) | (s, 1H), 6.71-6.63 (m, 2H), 6.50 (s, |
| | | | | 1H), 4.27 (s, 2H), 3.44 (q, J = 7.1 Hz, |
| | | | | 2H), 3.00 (s, 3H), 2.48 (s, 3H), 2.20 |
| | | | | (q, J = 7.6 Hz, 2H), 2.11 (s, 3H), 1.19 |
| | | | | (t, J = 7.0 Hz, 3H), 0.99 (t, J = 7.6 |
| | | | | Hz, 3H); |
| | | | | ¹⁹F NMR (471 MHz, CDCl₃) |
| | | | | δ −110.25 (td, J = 8.9, 4.5 Hz), −111.67 |
| | | | | (t, J = 6.5 Hz) |
| 88 | | | ESIMS m/z 389 | ¹H NMR (500 MHz, CDCl₃) δ 7.71 |
| | | | ([M + H]⁺) | (s, 1H), 6.72-6.63 (m, 2H), 6.53 (s, |
| | | | | 1H), 4.26 (s, 2H), 3.27 (t, J = 6.1 Hz, |
| | | | | 2H), 3.03 (s, 3H), 2.46 (s, 3H), 2.12 |
| | | | | (d, J = 2.2 Hz, 5H), 1.80 (dq, J = 8.3, |
| | | | | 6.0 Hz, 2H), 1.69-1.60 (m, 2H); |
| | | | | ¹⁹F NMR (471 MHz, CDCl₃) |
| | | | | δ −110.18-−110.30 |
| | | | | (m), −111.59-−111.73 (m) |
| 89 | | | ESIMS m/z 379 | ¹H NMR (500 MHz, CDCl₃) δ 7.66 |
| | | | ([M + H]⁺) | (s, 1H), 7.32 (s, 1H), 6.72-6.64 (m, |
| | | | | 3H), 4.25 (s, 2H), 3.90 (s, 3H), 3.65- |
| | | | | 3.27 (m, 2H), 3.11-2.98 (m, 3H), |
| | | | | 2.46 (s, 3H), 1.24 (t, J = 7.3 Hz, 3H); |
| | | | | ¹⁹F NMR (471 MHz, CDCl₃) |
| | | | | δ −109.86 (t, J = 7.5 Hz), −111.59 (q, |
| | | | | J = 6.7 Hz) |
| 90 | | | ESIMS m/z 315 | ¹H NMR (500 MHz, CDCl₃) δ 7.50 |
| | | | ([M + H]⁺) | (s, 1H), 6.45 (s, 1H), 3.45 (q, J = 7.1 |
| | | | | Hz, 2H), 3.00 (s, 3H), 2.90 (d, J = 7.2 |
| | | | | Hz, 2H), 2.45 (s, 3H), 2.33 (p, J = 8.2 |
| | | | | Hz, 1H), 2.07 (s, 3H), 1.89-1.80 (m, |
| | | | | 2H), 1.79 (s, 3H), 1.67-1.59 (m, |
| | | | | 2H), 1.58-1.49 (m, 2H), 1.23-1.10 |
| | | | | (m, 5H); |
| | | | | ¹³C NMR (126 MHz, CDCl₃) δ |
| | | | | 203.19, 155.13, 154.18, 137.61, |
| | | | | 131.69, 130.98, 126.57, 125.34, |
| | | | | 47.11, 44.64, 36.54, 35.32, 32.70, |
| | | | | 25.00, 21.60, 17.73, 15.10, 12.74 |
| 91 | | | ESIMS m/z 329 | ¹H NMR (500 MHz, CDCl₃) δ 7.51 |
| | | | ([M + H]⁺) | (s, 1H), 6.47 (s, 1H), 3.44 (q, J = 7.1 |
| | | | | Hz, 2H), 2.99 (s, 3H), 2.90 (d, J = 7.1 |
| | | | | Hz, 2H), 2.46 (s, 3H), 2.40-2.28 (m, |
| | | | | 1H), 2.19 (q, J = 7.6 Hz, 2H), 2.07 (s, |
| | | | | 3H), 1.89-1.79 (m, 2H), 1.68-1.48 |
| | | | | (m, 4H), 1.22-1.12 (m, 5H), 0.98 (t, |
| | | | | J = 7.6 Hz, 3H); |
| | | | | ¹³C NMR (126 MHz, CDCl₃) δ |
| | | | | 203.13, 159.52, 154.11, 137.55, |
| | | | | 131.69, 130.87, 126.33, 125.27, |
| | | | | 47.07, 44.36, 36.52, 35.09, 32.70, |
| | | | | 25.00, 21.63, 21.18, 17.84, 12.75, |
| | | | | 11.82 |
| 92 | | | ESIMS m/z 327 | ¹H NMR (500 MHz, CDCl₃) δ 7.51 |
| | | | ([M + H]⁺) | (s, 1H), 6.49 (s, 1H), 3.25 (t, J = 6.1 |
| | | | | Hz, 2H), 3.01 (s, 3H), 2.90 (d, J = 7.2 |
| | | | | Hz, 2H), 2.45 (s, 3H), 2.34 (hept, J = |
| | | | | 7.7 Hz, 1H), 2.11 (t, J = 6.6 Hz, 2H), |
| | | | | 2.08 (s, 3H), 1.82 (dp, J = 24.0, 6.4, |
| | | | | 5.7 Hz, 4H), 1.63 (qd, J = 6.5, 3.5 Hz, |
| | | | | 4H), 1.58-1.49 (m, 2H), 1.21-1.11 |
| | | | | (m, 2H); |
| | | | | ¹³C NMR (126 MHz, CDCl₃) δ |
| | | | | 202.92, 156.07, 153.72, 137.52, |
| | | | | 131.67, 130.91, 126.32, 125.25, |
| | | | | 50.58, 47.01, 37.13, 36.48, 32.67, |
| | | | | 27.00, 24.98, 23.68, 21.62, 21.39, |
| | | | | 17.66 |
| 93 | | | ESIMS m/z 395 | ¹H NMR (500 MHz, CDCl₃) δ 7.66 |
| | | | ([M + H]⁺) | (s, 1H), 7.10 (s, 1H), 6.87 (s, 1H), |
| | | | | 6.73-6.65 (m, 2H), 4.24 (s, 2H), |
| | | | | 3.87 (q, J = 7.1 Hz, 2H), 3.24 (s, 3H), |
| | | | | 2.43 (s, 3H), 2.30 (s, 3H), 1.29 (t, J = |
| | | | | 7.2 Hz, 3H); |
| | | | | ¹⁹F NMR (471 MHz, CDCl₃) |
| | | | | δ −109.45 (tt, J = 10.0, 5.8 Hz), −111.65 |
| | | | | (p, J = 7.0 Hz) |
| 94 | | | ESIMS m/z 333 | ¹H NMR (500 MHz, CDCl₃) δ 7.41 |
| | | | ([M + H]⁺) | (s, 1H), 7.02 (s, 2H), 3.87 (q, J = 7.1 |
| | | | | Hz, 2H), 3.25 (s, 3H), 2.88 (d, J = 7.1 |
| | | | | Hz, 2H), 2.38 (s, 3H), 2.36-2.28 (m, |
| | | | | 1H), 2.24 (s, 3H), 1.89-1.80 (m, |
| | | | | 2H), 1.68-1.49 (m, 4H), 1.29 (t, J = |
| | | | | 7.1 Hz, 3H), 1.20-1.09 (m, 2H) |
| 95 | | | ESIMS m/z 259 | ¹H NMR (500 MHz, CDCl₃) δ 7.70 |
| | | | ([M + H]⁺) | (d, J = 2.1 Hz, 1H), 7.65 (dd, J = |
| | | | | 8.2, 2.1 Hz, 1H), 7.58-7.36 (m, |
| | | | | 1H), 6.75 (d, J = 8.1 Hz, 1H), 3.97 |
| | | | | (pd, J = 8.5, 1.1 Hz, 1H), 3.58-3.24 |
| | | | | (m, 2H), 3.02 (s, 3H), 2.46-2.35 |
| | | | | (m, 2H), 2.29 (s, 3H), 2.28-2.23 |
| | | | | (m, 2H), 2.06 (dp, J = 11.0, 8.7 Hz, |
| | | | | 1H), 1.95-1.83 (m, 1H), 1.22 (t, J = |
| | | | | 7.2 Hz, 3H) |
| 96 | | | ESIMS m/z 277 | ¹H NMR (500 MHz, CDCl₃) δ 7.67 |
| | | | ([M + H]⁺) | (d, J = 8.4 Hz, 1H), 7.56-7.37 (m, |
| | | | | 1H), 6.44 (d, J = 12.9 Hz, 1H), 3.93- |
| | | | | 3.82 (m, 1H), 3.60-3.22 (m, 2H), |
| | | | | 3.03 (s, 3H), 2.40-2.32 (m, 2H), |
| | | | | 2.30-2.18 (m, 5H), 2.00 (dp, J = |
| | | | | 10.9, 8.6 Hz, 1H), 1.91-1.79 (m, |
| | | | | 1H), 1.27-1.19 (m, 3H); |
| | | | | ¹⁹F NMR (471 MHz, CDCl₃) |
| | | | | δ −113.81 |
| 97 | | | ESIMS m/z 273 | ¹H NMR (500 MHz, Acetone-d₆) δ |
| | | | ([M + H]⁺) | 7.95-7.75 (m, 3H), 7.03-6.96 (m, |
| | | | | 2H), 3.59-3.38 (m, 2H), 3.05 (m, |
| | | | | 3H), 2.95 (d, J = 7.1 Hz, 2H), 2.40- |
| | | | | 2.28 (m, 1H), 1.89-1.79 (m, 2H), |
| | | | | 1.68-1.59 (m, 2H), 1.59-1.49 (m, |
| | | | | 2H), 1.26-1.14 (m, 5H) |
| 98 | | | ESIMS m/z 327 | ¹H NMR (500 MHz, CDCl₃) δ 7.56- |
| | | | ([M + H]⁺) | 7.37 (m, 1H), 7.24 (s, 1H), 7.03 (s, |
| | | | | 1H), 3.89-3.77 (m, 1H), 3.58- |
| | | | | 3.29 (m, 2H), 3.03 (s, 3H), 2.42- |
| | | | | 2.29 (m, 5H), 2.24-2.17 (m, 2H), |
| | | | | 1.99 (dp, J = 10.9, 8.9 Hz, 1H), 1.92- |
| | | | | 1.82 (m, 1H), 1.27-1.21 (m, 3H); |
| | | | | ¹⁹F NMR (471 MHz, CDCl₃) δ −57.86 |
| 99 | | | ESIMS m/z 289 | ¹H NMR (500 MHz, CDCl₃) δ 7.57 |
| | | | ([M + H]⁺) | (s, 1H), 7.47 (s, 1H), 6.28 (s, 1H), |
| | | | | 3.97 (pd, J = 8.6, 1.2 Hz, 1H), 3.85 (s, |
| | | | | 3H), 3.43 (d, J = 97.5 Hz, 2H), 3.02 |
| | | | | (s, 3H), 2.38-2.25 (m, 2H), 2.25- |
| | | | | 2.14 (m, 5H), 1.95 (dp, J = 10.9, 8.7 |
| | | | | Hz, 1H), 1.89-1.76 (m, 1H), 1.23 (t, |
| | | | | J = 7.1 Hz, 3H) |
| 100 | | | ESIMS m/z 327 | ¹H NMR (500 MHz, CDCl₃) δ 7.82 |
| | | | ([M + H]⁺) | (s, 1H), 7.59-7.39 (m, 1H), 6.71 (d, |
| | | | | J = 5.5 Hz, 1H), 3.92 (pd, J = 8.6, |
| | | | | 1.1 Hz, 1H), 3.62-3.27 (m, 2H), |
| | | | | 3.03 (s, 3H), 2.57 (s, 3H), 2.42- |
| | | | | 2.31 (m, 2H), 2.27-2.21 (m, 2H), |
| | | | | 2.06 (dp, J = 11.0, 8.9 Hz, 1H), 1.95- |
| | | | | 1.81 (m, 1H), 1.27-1.17 (m, 3H); |
| | | | | ¹⁹F NMR (471 MHz, CDCl₃) |
| | | | | δ −60.88 |
| 101 | | | ESIMS m/z 259 | ¹H NMR (500 MHz, CDCl₃) δ 7.62 |
| | | | ([M + H]⁺) | (s, 1H), 7.57-7.52 (m, 1H), 6.87- |
| | | | | 6.60 (m, 2H), 3.95-3.94 (m, 1H), |
| | | | | 3.55-3.28 (m, 2H), 3.02 (s, 3H), |
| | | | | 2.55 (s, 3H), 2.43-2.31 (m, 2H), |
| | | | | 2.27-2.15 (m, 2H), 2.02 (dp, J = |
| | | | | 11.1, 8.8 Hz, 1H), 1.92-1.80 (m, |
| | | | | 1H), 1.22 (t, J = 7.2 Hz, 3H) |
| 102 | | | ESIMS m/z 277 | ¹H NMR (500 MHz, CDCl₃) δ 7.73- |
| | | | ([M + H]⁺) | 7.57 (m, 1H), 7.30 (d, J = 12.2 Hz, |
| | | | | 1H), 6.78 (d, J = 8.4 Hz, 1H), 3.86 |
| | | | | (pd, J = 8.6, 1.1 Hz, 1H), 3.59-3.26 |
| | | | | (m, 2H), 3.06-3.00 (m, 3H), 2.50 |
| | | | | (s, 3H), 2.42-2.31 (m, 2H), 2.30- |
| | | | | 2.18 (m, 2H), 2.03 (dp, J = 11.0, 8.8 |
| | | | | Hz, 1H), 1.87 (ddddd, J= 10.6, 9.2, |
| | | | | 7.9, 3.9, 1.1 Hz, 1H), 1.23 (q, J = 8.7 |
| | | | | Hz, 3H); |
| | | | | ¹⁹F NMR (471 MHz, CDCl₃) −132.99 |
| 103 | | | ESIMS m/z 289 | ¹H NMR (500 MHz, CDCl₃) δ 7.67- |
| | | | ([M + H]⁺) | 7.47 (m, 1H), 7.10 (s, 1H), 6.64 (s, |
| | | | | 1H), 3.96-3.85 (m, 1H), 3.84 (s, |
| | | | | 3H), 3.61-3.27 (m, 2H), 3.06-2.97 |
| | | | | (m, 3H), 2.48 (s, 3H), 2.45-2.32 (m, |
| | | | | 2H), 2.24 (tdd, J = 13.9, 7.9, 4.0 Hz, |
| | | | | 2H), 2.04 (dp, J = 11.0, 8.9 Hz, 1H), |
| | | | | 1.93-1.81 (m, 1H), 1.22 (t, J = 7.2 |
| | | | | Hz, 3H) |
| 104 | | | ESIMS m/z 349 | ¹H NMR (500 MHz, CDCl₃) δ 7.84 |
| | | | ([M + H]⁺) | (d, J = 2.1 Hz, 1H), 7.80 (dd, J = 8.3, |
| | | | | 2.1 Hz, 1H), 7.49 (m, 1H), 6.80 (d, |
| | | | | J = 8.2 Hz, 1H), 6.68 (t, J = 8.1 Hz, |
| | | | | 2H), 4.28 (s, 2H), 3.61-3.28 (m, |
| | | | | 2H), 3.04 (s, 3H), 2.32 (s, 3H), 1.24 |
| | | | | (t, J = 7.3 Hz, 3H); |
| | | | | ¹⁹F NMR (471 MHz, CDCl₃) |
| | | | | δ −110.11 (t, J = 7.3 Hz), −111.55 (t, |
| | | | | J = 6.8 Hz) |
| 105 | | | ESIMS m/z 303 | ¹H NMR (500 MHz, CDCl₃) δ 7.37 |
| | | | ([M + H]⁺) | (s, 1H), 6.41 (s, 1H), 3.35 (s, 2H), |
| | | | | 2.99 (s, 3H), 2.61 (s, 2H), 2.17 (s, |
| | | | | 3H), 2.16 (s, 3H), 2.12 (s, 3H), 1.20 |
| | | | | (t, J = 7.1 Hz, 3H), 1.12 (s, 9H) |

TABLE 3
Biological Testing Rating Scale
Rating Table for Fungal Pathogens

| % Disease Control | Rating |
|---|---|
| >85% | A |
| 50-85% | B |
| 20-<50% | C |
| <20% | D |
| Not Tested | E |

TABLE 4
Biological Activity - COCHSA, PHAKPA, PUCCRT, RHYNSE,
and SEPTTR Disease Control in Applications at 200 ppm

| | % Disease Control Rating | | | | | | |
|---|---|---|---|---|---|---|---|
| | CD-1DP | | | | | CD-3DC | |
| Cmpd. | COCHSA | PHAKPA | PUCCRT | RHYNSE | SEPTTR | PHAKPA | SEPTTR |
| No. | 200 ppm | 200 ppm | 200 ppm | 200 ppm | 200 ppm | 200 ppm | 200 ppm |
| 1 | E | A | A | A | D | A | C |
| 2 | E | A | A | A | C | A | D |
| 3 | E | A | A | A | A | A | C |
| 4 | A | A | A | A | A | A | B |
| 5 | E | A | A | A | C | A | D |
| 6 | E | A | A | A | A | A | C |
| 7 | E | A | A | A | C | A | D |
| 8 | E | A | A | A | B | A | C |
| 9 | A | A | A | A | D | A | D |
| 10 | E | A | D | C | D | A | D |
| 11 | E | A | D | D | D | A | D |
| 12 | E | B | D | D | D | B | D |
| 13 | E | B | D | D | D | A | D |
| 14 | E | A | A | D | D | A | D |
| 15 | E | A | D | B | D | A | D |
| 16 | E | B | D | D | D | A | D |
| 17 | E | A | A | A | B | A | D |
| 18 | E | A | B | A | B | A | D |
| 19 | E | A | A | C | D | A | C |
| 20 | E | A | A | A | D | A | D |
| 21 | E | A | A | A | B | A | D |
| 22 | E | A | A | A | D | A | D |
| 23 | E | A | A | A | C | A | D |
| 24 | E | D | A | D | D | D | D |
| 25 | E | A | A | A | C | A | D |
| 26 | E | A | A | A | A | A | C |
| 27 | E | A | A | A | A | A | B |
| 29 | E | A | A | A | B | A | B |
| 30 | E | A | B | D | D | A | D |
| 31 | E | D | A | B | A | D | D |
| 32 | E | A | A | A | A | A | D |
| 33 | E | A | A | A | A | A | B |
| 34 | E | A | A | A | B | A | D |
| 35 | E | A | A | A | B | A | D |
| 36 | E | A | A | B | E | A | D |
| 37 | E | A | A | D | A | A | B |
| 38 | E | D | D | D | D | D | D |
| 39 | E | B | E | E | E | A | E |
| 40 | E | D | E | E | E | D | E |
| 41 | E | A | E | E | E | A | E |
| 42 | E | A | E | E | E | A | E |
| 43 | E | D | E | E | E | D | E |
| 44 | E | D | E | E | E | D | E |
| 45 | E | C | A | D | D | B | D |
| 46 | E | C | D | D | D | D | D |
| 47 | E | A | A | C | A | A | D |
| 48 | E | D | D | D | D | D | D |
| 49 | E | A | A | D | C | B | D |
| 50 | E | A | A | D | A | A | C |
| 51 | E | D | D | D | C | D | D |
| 52 | E | A | A | D | C | B | D |
| 53 | E | D | D | D | D | D | D |
| 54 | E | A | A | D | A | B | D |
| 55 | E | A | A | D | B | A | D |
| 56 | E | D | C | D | D | D | D |
| 57 | E | A | A | D | B | A | D |
| 58 | E | A | A | D | B | A | D |
| 59 | E | A | A | A | A | A | A |
| 60 | E | D | A | D | D | D | D |
| 61 | E | A | A | A | A | A | D |
| 62 | E | A | A | A | A | C | B |
| 63 | E | A | A | A | A | A | C |
| 64 | E | A | A | A | B | A | D |
| 65 | E | A | A | A | A | A | C |
| 66 | E | A | A | A | A | A | D |
| 67 | E | A | A | A | A | A | D |
| 68 | E | A | A | A | A | A | D |
| 69 | E | A | A | B | B | A | B |
| 70 | E | A | A | A | B | A | D |
| 71 | E | A | A | A | C | A | D |
| 72 | E | A | A | D | D | A | D |
| 73 | E | A | A | D | D | A | D |
| 74 | E | A | A | B | D | A | D |
| 75 | E | C | A | D | D | A | D |
| 76 | E | A | A | D | D | E | D |
| 77 | E | A | A | C | D | A | D |
| 78 | E | A | A | D | D | A | D |
| 79 | E | A | A | B | D | E | D |
| 80 | E | A | A | A | B | A | D |
| 81 | E | B | A | D | D | E | D |
| 82 | E | A | A | A | A | A | D |
| 83 | E | A | A | A | D | A | D |
| 84 | E | A | A | A | D | A | D |
| 85 | E | A | A | A | A | A | C |
| 86 | E | A | A | D | D | A | D |
| 87 | E | B | A | D | D | C | D |
| 88 | E | B | B | D | D | A | D |
| 89 | E | B | A | C | D | C | D |
| 90 | E | A | A | C | D | A | D |
| 91 | E | A | A | D | D | A | D |
| 92 | E | B | A | C | D | B | D |
| 93 | E | A | A | A | B | A | D |
| 94 | E | A | A | A | A | A | D |
| 95 | E | C | A | E | E | C | E |
| 97 | E | B | B | E | E | B | E |
| 98 | E | C | D | E | E | C | E |
| 99 | E | A | A | E | E | A | E |
| 100 | E | A | A | E | E | A | E |
| 101 | E | D | D | E | E | D | E |
| 102 | E | B | A | E | E | A | E |
| 103 | E | C | B | E | E | A | E |
| 104 | E | B | A | E | E | A | E |
| 105 | E | E | E | E | E | E | E |
| 106 | E | E | E | E | E | E | E |

*Cmpd. No.—Compound Number
*COCHSA—Spot Blotch of Barley (Cochliobolus sativus)
*PHAKPA—Asian Soybean Rust (Phakopsora pachyrhizi)
*PUCCRT—Wheat Brown Rust (Puccinia triticina)
*RHYNSE—Leaf Blotch of Barley (Rhynochosporium commune)
*SEPTTR—Septoria Leaf Blotch of Wheat (Zymoseptoria tritici)
*1DP—1 Day Protectant
*3DC—3 Day Curative
*ppm—Parts Per Million

1. A compound of Formula I: 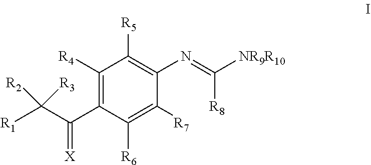
wherein
R₁ is selected from the group consisting of C₂-C₈ alkyl, C₁-C₈ haloalkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, C₃-C₈ heterocycloalkyl, C₃-C₈ substituted heterocycloalkyl, C₅-C₇ heteroaryl, C₅-C₇ substituted heteroaryl, aryl, and substituted aryl;
each R₂ and R₃ independently is selected from the group consisting of hydrogen, C₁-C₈ alkyl, C₁-C₈ haloalkyl, C₂-C₈ alkenyl, C₂-C₈ substituted alkenyl, C₂-C₈ alkynyl, C₂-C₈ substituted alkynyl;
or R₁ and R₂ may be covalently bonded together to form a C₄-C₈ cycloalkyl group, C₃-C₈ substituted cycloalkyl group, C₃-C₈ heterocycloalkyl, or C₃-C₈ substituted heterocycloalkyl group;
each R₄, R₅, and R₆ independently is selected from the group consisting of hydrogen, halogen, cyano, nitro, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, and C₁-C₈ substituted alkoxy;
R₇ is H;
R₈ is selected from the group consisting of hydrogen, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈alkoxy, and C₁-C₈ substituted alkoxy;
or R₈ and R₉ may be covalently bonded together to form a C₃-C₈ heterocycloalkyl or C₃-C₈ substituted heterocycloalkyl group;
each R₉ and R₁₀ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, aryl, substituted aryl, C₁-C₈ alkylaryl, and substituted C₁-C₈ alkylaryl;
or R₉ and R₁₀ may be covalently bonded together to form a C₃-C₈ heterocycloalkyl or C₃-C₈ substituted heterocycloalkyl group;
X is O;
wherein any and all heterocyclic rings may contain up to three heteroatoms selected from the group consisting of O, N, and S;
or a tautomer or salt thereof.

2. The compound of claim 1, wherein R₁ is selected from the group consisting of C₂-C₈ alkyl, C₁-C₈ haloalkyl, C₃-C₈ cycloalkyl, C₃-C₈ substituted cycloalkyl, aryl, and substituted aryl.

3. The compound of claim 1, wherein R₂ and R₃ are both hydrogen.

4. The compound of claim 1, wherein R₄ is hydrogen.

5. The compound of claim 4, wherein each R₅ and R₆ independently is selected from the group consisting of halogen, C₁-C₈ alkyl, C₁-C₈ substituted alkyl, and C₁-C₈ alkoxy.

6. The compound of claim 5, wherein R₅ and R₆ are both CH₃.

7. The compound of claim 1, wherein R₆ is hydrogen.

8. The compound of claim 7, wherein each R₄ and R₅ independently is selected from the group consisting of halogen, C₁-C₈ alkyl, C₁-C₈ substituted alkyl, and C₁-C₈ alkoxy.

9. The compound of claim 8, wherein R₄ and R₅ are both CH₃.

10. The compound of claim 1, wherein each R₉ and R₁₀ independently is selected from the group consisting of C₁-C₈ alkyl, C₁-C₈ substituted alkyl, C₂-C₈ alkenyl, C₃-C₈ cycloalkyl, aryl, substituted aryl, C₁-C₈ alkylaryl, and substituted C₁-C₈ alkylaryl.

11. The compound of claim 1, wherein R₈ is selected from the group consisting of hydrogen, C₁-C₈ alkyl, and C₁-C₈ substituted alkyl.

12. A compound wherein the compound is selected from one of the compounds in Table 1.

13. A fungicidal compound of Formula I. 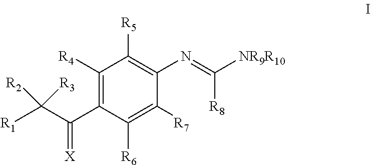
wherein
R₁ is selected from the group consisting of C₂-C₈ alkyl, C₁-C₈ haloalkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, C₃-C₈ heterocycloalkyl, C₃-C₈ substituted heterocycloalkyl, C₅-C₇ heteroaryl, C₅-C₇ substituted heteroaryl, aryl; and substituted aryl;
each R₂ and R₃ independently is selected from the group consisting of hydrogen, C₁-C₈ alkyl, C₁-C₈ haloalkyl, C₂-C₈ alkenyl, C₂-C₈ substituted alkenyl, C₂-C₈ alkynyl, C₂-C₈ substituted alkynyl;
or R₁ and R₂ may be covalently bonded together to form a C₄-C₈ cycloalkyl group, C₃-C₈ substituted cycloalkyl group, C₃-C₈ heterocycloalkyl, or C₃-C₈ substituted heterocycloalkyl group;
each R₄, R₅, and R₆ independently is selected from the group consisting of hydrogen, halogen, cyano, nitro, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, and C₁-C₈ substituted alkoxy;
R₇ is H;
R₈ is selected from the group consisting of hydrogen, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₁-C₈ alkoxy, C₁-C₈ substituted alkoxy, and thiol;
or R₈ and R₉ may be covalently bonded together to form a C₃-C₈ heterocycloalkyl or C₃-C₈ substituted heterocycloalkyl group;
each R₉ and R₁₀ independently is selected from the group consisting of C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₃-C₈ cycloalkyl, C₃-C₈ substituted cycloalkyl, aryl, substituted aryl, C₁-C₈ alkylaryl, and substituted C₁-C₈ alkylaryl;
or R₉ and R₁₀ may be covalently bonded together to form a C₃-C₈ heterocycloalkyl or C₃-C₈ substituted heterocycloalkyl group;
X is O;
wherein any and all heterocyclic rings may contain up to three heteroatoms selected from the group consisting of O, N, and S;
or a tautomer or salt thereof, for controlling a fungal pathogen.

14. The fungicidal compound of claim 13, wherein the fungal pathogen is one of Zymoseptoria tritici, Cochliobolus sativus, Puccinia triticina, Puccinia striiformis, Venturia inaequalis, Ustilago maydis, Uncinula necator, Rhynchosporium commune, Magnaporthe grisea, Phakopsora pachyrhizi, Parastagonospora nodorum, Glomerella lagenarium, Cercospora beticola, Alternaria solani, Pyrenophora teres, Blumeria graminis f sp. tritici, Blumeria graminis f sp. hordei, Erysiphe cichoracearum, Fusarium virguliforme, Rhizoctonia solani, Pythium ultimum, Botrytis cinerea, Ramularia collo-cygni, Pyrenophora tritici-repentis, Exserohilum turcicum, Puccinia polysora, Sclerotinia sclerotiorum, Erysiphe diffusa, Fusarium graminearum, Podosphaera leucotricha, Colletotrichum truncatum, Cercospora kikuchii, Cerospora sojina, Corynespora cassiicola, and Septoria glycines.

15. The fungicidal compound of claim 13, wherein the compound treats one of the following diseases from the fungal pathogen: Septoria Leaf Blotch of Wheat (Zymoseptoria tritici), Spot Blotch of Barley (Cochliobolus sativus), Wheat Brown Rust (Puccinia triticina), Stripe Rust of Wheat (Puccinia striiformis), Scab of Apple (Venturia inaequalis), Blister Smut of Maize (Ustilago maydis), Powdery Mildew of Grapevine (Uncinula necator), Leaf Blotch of Barley (Rhynchosporium commune), Blast of Rice (Magnaporthe grisea), Asian Soybean Rust (Phakopsora pachyrhizi), Glume Blotch of Wheat (Parastagonospora nodorum), Anthracnose of Cucurbits (Glomerella lagenarium), Leaf Spot of Beet (Cercospora beticola), Early Blight of Tomato (Alternaria solani), Net Blotch of Barley (Pyrenophora teres), Powdery Mildew of Wheat (Blumeria graminis f sp. tritici), Powdery Mildew of Barley (Blumeria graminisf sp. hordei), Powdery Mildew of Cucurbits (Erysiphe cichoracearum), Sudden Death Syndrome of Soybean (Fusarium virguiforme), Collar Rot or Damping-Off of Seedlings (Rhizoctonia solani), Root Rot (Pythium ultimum), Grey Mold (Botrytis cinerea), Ramularia Leaf Spot (Ramularia collo-cygni), Tan Spot of Wheat (Pyrenophora tritici-repentis), Northern Leaf Blight of Maize (Exserohilum turcicum), Southern Rust of Maize (Puccinia polysora), White Mold (Sclerotinia sclerotiorum), Powdery Mildew of Soybean (Erysiphe diffusa), Head Blight of Cereals (Fusarium graminearum), Powdery Mildew of Apple (Podosphaera leucotricha), Anthracnose of Soybean (Colletotrichum truncatum), Cercospora Leaf Blight (Cercospora kikuchii), Frogeye Leaf Spot (Cerospora sojina), Target Spot of Soybean (Corynespora cassiicola), and Leaf Spot of Soybean (Septoria glycines).

16. A composition for use in the control of a fungal pathogen, the composition comprising a phytologically acceptable amount of the compound of claim 1 and a carrier.

17. The composition of claim 16, wherein the carrier is one or more of a thickener, emulsifier, rheology agent, dispersant and polymer.

18. The composition of claim 16, wherein the fungal pathogen is one of Zymoseptoria tritici, Cochliobolus sativus, Puccinia triticina, Puccinia striiformis, Venturia inaequalis, Ustilago maydis, Uncinula necator, Rhynchosporium commune, Magnaporthe grisea, Phakopsora pachyrhizi, Parastagonospora nodorum, Glomerella lagenarium, Cercospora beticola, Alternaria solani, Pyrenophora teres, Blumeria graminis f sp. tritici, Blumeria graminis f sp. hordei, Erysiphe cichoracearum, Fusarium virgulforme, Rhizoctonia solani, Pythium ultimum, Botrytis cinerea, Ramularia collo-cygni, Pyrenophora tritici-repentis, Exserohilum turcicum, Puccinia polysora, Sclerotinia sclerotiorum, Erysiphe diffusa, Fusarium graminearum, Podosphaera leucotricha, Colletotrichum truncatum, Cercospora kikuchii, Cerospora sojina, Corynespora cassiicola, and Septoria glycines.

19. The composition of claim 16 wherein the composition treats one of the following diseases from the fungal pathogen: Septoria Leaf Blotch of Wheat (Zymoseptoria tritici), Spot Blotch of Barley (Cochliobolus sativus), Wheat Brown Rust (Puccinia triticina), Stripe Rust of Wheat (Puccinia striiformis), Scab of Apple (Venturia inaequalis), Blister Smut of Maize (Ustilago maydis), Powdery Mildew of Grapevine (Uncinula necator), Leaf Blotch of Barley (Rhynchosporium commune), Blast of Rice (Magnaporthe grisea), Asian Soybean Rust (Phakopsora pachyrhizi), Glume Blotch of Wheat (Parastagonospora nodorum), Anthracnose of Cucurbits (Glomerella lagenarium), Leaf Spot of Beet (Cercospora beticola), Early Blight of Tomato (Alternaria solani), Net Blotch of Barley (Pyrenophora teres), Powdery Mildew of Wheat (Blumeria graminis f sp. tritici), Powdery Mildew of Barley (Blumeria graminis f sp. hordei), Powdery Mildew of Cucurbits (Erysiphe cichoracearum), Sudden Death Syndrome of Soybean (Fusarium virguliforme), Collar Rot or Damping-Off of Seedlings (Rhizoctonia solani), Root Rot (Pythium ultimum), Grey Mold (Botrytis cinerea), Ramularia Leaf Spot (Ramularia collo-cygni), Tan Spot of Wheat (Pyrenophora tritici-repentis), Northern Leaf Blight of Maize (Exserohilum turcicum), Southern Rust of Maize (Puccinia polysora), White Mold (Sclerotinia sclerotiorum), Powdery Mildew of Soybean (Erysiphe diffusa), Head Blight of Cereals (Fusarium graminearum), Powdery Mildew of Apple (Podosphaera leucotricha), Anthracnose of Soybean (Colletotrichum truncatum), Cercospora Leaf Blight (Cercospora kikuchii), Frogeye Leaf Spot (Cerospora sojina), Target Spot of Soybean (Corynespora cassiicola), and Leaf Spot of Soybean (Septoria glycines).

20. The composition of claim 19, wherein the disease is one of Septoria Leaf Blotch of Wheat, Spot Blotch of Barley, Leaf Blotch of Barley, Wheat Brown Rust, and Asian Soybean Rust.

21. A seed treated with a phytologically acceptable amount of the compound of claim 1.

22. A method of controlling fungal attack on a plant, the method comprising contacting an area adjacent to the plant, soil adapted to support growth of the plant, a root of the plant, and foliage of the plant, with a phytologically acceptable amount of the compound of claim 1.

23. A seed treated with a phytologically acceptable amount of the composition of claim 16.

24. A method of controlling fungal attack on a plant, the method comprising contacting an area adjacent to the plant, soil adapted to support growth of the plant, a root of the plant, and foliage of the plant, with a phytologically acceptable amount of the composition of claim 16.
